(19) United States
(12) Patent Application Publication
(10) Pub. No.: US 2008/0312240 A1
(43) Pub. Date: Dec. 18, 2008

(54) Bicyclic Piperazines as Metabotropic Glutatmate Receptor Antagonists

(75) Inventors: Louise Edwards, Mississauga (CA); Methvin Isaac, Brampton (CA); Abdelmalik Slassi, Mississauga (CA); Guang-Ri Sun, Markham (CA); Tao Xin, Woodbridge (CA); Alexander Minidis, Sodertalje (SE); Peter Dove, Toronto (CA)

Correspondence Address: BIRCH STEWART KOLASCH & BIRCH, PO BOX 747, FALLS CHURCH, VA, 22040-0747, US

(73) Assignee: AstraZeneca AB, SODERTALJE (SE)

(21) Appl. No.: 11/997,541

(22) PCT Filed: Aug. 4, 2006
(86) PCT No.: PCT/US06/30393

§ 371 Date: Apr. 28, 2008

Related U.S. Application Data
(60) Provisional application No. 60/707,945, filed on Aug. 15, 2005.

(51) Int. Cl. A61K 31/4985; C07D 401/14; C07D 403/14; A61P 25/00; A61P 1/00; C07D 413/14; C07D 471/04

(52) U.S. Cl. 514/249; 544/349

(57) ABSTRACT

The invention relates to compounds of formula I or pharmaceutically acceptable salts or solvates thereof:

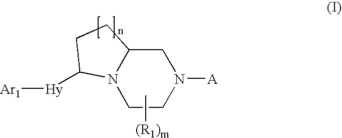

where Ar₁, A, Hy, R₁, m and n are as defined in the description. The invention also includes pharmaceutical compositions and uses of, and processes of making the compounds, as well as methods of medical treatment of mGluR5-mediated disorders.

### FIELD OF THE INVENTION

The present invention relates to a new class of compounds, to pharmaceutical formulations containing said compounds and to the use of said compounds in therapy. The present invention further relates to the process for the preparation of said compounds and to new intermediates prepared therein.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

### Neurological, Psychiatric and Pain Disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors, and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants. Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K, Holloway, R. H., Penagini, R., Blackshaw, L. A., Dent, J., 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000; Diagnosis of reflux disease. Baillière's Clin. Gastroenterol. 14, pp. 759-774.

Because of their physiological and pathophysiological significance, there is a need for new potent mGluR agonists and antagonists that display a high selectivity for mGluR subtypes, particularly the Group I receptor subtype, most particularly the mGluR5

The object of the present invention is to provide compounds exhibiting an activity at metabotropic glutamate receptors (mGluRs), especially at the mGluR5 receptor.

### SUMMARY OF THE INVENTION

One embodiment of the invention relates to compounds of formula I:

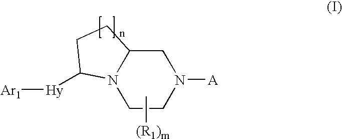

wherein:
Ar₁ is an optionally-substituted, aryl or heteroaryl group, wherein the substituents are selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R², SO₂R², aryl, heteroaryl, cycloalkyl and heterocycloalkyl, wherein any cyclic group may be further substituted with at least one substituent selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R² and SO₂R²;
A is selected from the group consisting of Ar₁, CO₂R², CONR²R³, S(O)R² and SO₂R²;
R₁, in each instance, is independently selected from the group consisting of F, Cl, Br, I, OH, CN, nitro, C₁₋₆-alkyl, OC₁₋₆-alkyl, C₁₋₆-alkylhalo, OC₁₋₆-alkylhalo, (CO)R², O(CO)R², O(CO)OR², CO₂R², —CONR²R³, C₁₋₆-alkyleneOR², OC₂₋₆-alkyleneOR² and C₁₋₆-alkylenecyano;
R² and R³ are independently selected from the group consisting of H, C₁₋₆-alkyl, C₁₋₆-alkylhalo, C₂₋₆-alkenyl, C₂₋₆-alkynyl and cycloalkyl;
Hy is a 5-membered heterocyclic ring containing two, three or four heteroatoms independently selected from the group consisting of N, O and S, wherein the ring is optionally substituted with one or more substituents selected from the group consisting of F, Cl, Br, I, OH, nitro, C₁₋₆-alkyl, C₁₋₆-alkylhalo, OC₁₋₆-alkyl, OC₁₋₆-alkylhalo, CN, CO₂R², NR²R³, SR², S(O)R² and SO₂R²;
m is an integer selected from the group consisting of 0, 1, 2, 3 and 4; and
n is an integer selected from the group consisting of 1, 2 and 3;
or a pharmaceutically-acceptable salt, hydrate, solvate, isoform, tautomer, optical isomer, or combination thereof.

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGlurR5 receptors, comprising treating a cell containing said receptor with an effective amount of the compound according to formula I.

### DETAILED DESCRIPTION OF THE PREFERRED EMBODIMENTS

The present invention is based upon the discovery of compounds that exhibit activity as pharmaceuticals, in particular as antagonists of metabotropic glutamate receptors. More particularly, the compounds of the present invention exhibit activity as antagonists of the mGluR5 receptor and, therefore, are useful in therapy, in particular for the treatment of neurological, psychiatric, pain, and gastrointestinal disorders associated with glutamate dysfunction.

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “alkyl” as used herein means a straight- or branched-chain hydrocarbon radical having from one to six carbon atoms, and includes methyl, ethyl, propyl, isopropyl, t-butyl and the like.

The term “alkenyl” as used herein means a straight- or branched-chain alkenyl radical having from two to six carbon atoms, and includes ethenyl, 1-propenyl, 1-butenyl and the like.

The term “alkynyl” as used herein means a straight- or branched-chain alkynyl radical having from two to six carbon atoms, and includes 1-propynyl (propargyl), 1-butynyl and the like.

The term “cycloalkyl” as used herein means a cyclic group (which may be unsaturated) having from three to seven carbon atoms, and includes cyclopropyl, cyclohexyl, cyclohexenyl and the like.

The term “heterocycloalkyl” as used herein means a three- to seven-membered cyclic group (which may be unsaturated) having at least one heteroatom selected from the group consisting of N, S and O, and includes piperidinyl, piperazinyl, pyrrolidinyl, tetrahydrofuranyl and the like.

The term “alkoxy” as used herein means a straight- or branched-chain alkoxy radical having from one to six carbon atoms and includes methoxy, ethoxy, propyloxy, isopropyloxy, t-butoxy and the like.

The term “halo” as used herein means halogen and includes fluoro, chloro, bromo, iodo and the like, in both radioactive and non-radioactive forms.

The term “alkylene” as used herein means a difunctional branched or unbranched saturated hydrocarbon radical having one to six carbon atoms, and includes methylene, ethylene, n-propylene, n-butylene and the like.

The term “alkenylene” as used herein means a difunctional branched or unbranched hydrocarbon radical having two to six carbon atoms and having at least one double bond, and includes ethenylene, n-propenylene, n-butenylene and the like.

The term “alkynylene” as used herein means a difunctional branched or unbranched hydrocarbon radical having two to six carbon atoms and having at least one triple bond, and includes ethynylene, n-propynylene, n-butynylene and the like.

The term “aryl” as used herein means an aromatic group having five to twelve atoms, and includes phenyl, naphthyl and the like.

The term “heteroaryl” means an aromatic group which includes at least one heteroatom selected from the group consisting of N, S and O, and includes groups and includes pyridyl, indolyl, furyl, benzofuryl, thienyl, benzothienyl, quinolyl, oxazolyl and the like.

The term “cycloalkenyl” as used herein means an unsaturated cycloalkyl group having from four to seven carbon atoms, and includes cyclopent-1-enyl, cyclohex-1-enyl and the like.

The terms “alkylaryl”, “alkylheteroaryl” and “alkylcycloalkyl” refer to an alkyl radical substituted with an aryl, heteroaryl or cycloalkyl group, and includes 2-phenethyl, 3-cyclohexyl propyl and the like.

The term “5-membered heterocyclic ring containing two or three heteroatoms independently selected from the group consisting of N, O and S” includes aromatic and heteroaromatic rings, as well as rings which may be saturated or unsaturated, and includes isoxazolyl, oxazolyl, oxadiazolyl, pyrazolyl, thiazolyl, imidazolyl, triazolyl and the like.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

“Solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents are ethanol, water and the like. When water is the solvent, the molecule is referred to as a hydrate.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound which is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

### Compounds

Compounds of the invention conform generally to formula I:

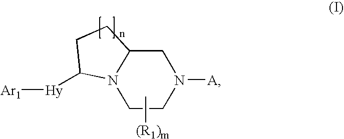

wherein Ar, Hy, L, R₁, m and n are defined hereinabove.

In one embodiment, Ar₁ is an optionally-substituted phenyl group; illustrative substituents may be selected from the group consisting of F, Cl, Br, nitro, C₁₋₆-alkyl, C₁₋₆-alkylhalo, OC₁₋₆-alkyl, OC₁₋₆-alkylhalo, and CN.

In another embodiment, A is an optionally-substituted pyridyl group, for example a 2-pyridyl group; illustrative substituents may be selected from the group consisting of F, Cl, Br, nitro, C₁₋₆-alkyl, C₁₋₆-alkylhalo, OC₁₋₆-alkyl, OC₁₋₆-alkylhalo, and CN.

In one embodiment Hy is an oxazole group; in another it is an isoxazole group; in yet others it is an oxadiazole group or a triazole group.

In still another embodiment, R¹ can be selected from the group consisting of C₁₋₆-alkyl, C₁₋₆-haloalkyl, —CN, —CO₂R², —CONR²R³, and —C₁₋₆alkyleneOR².

In one embodiment, n is 1; in another n is 2.

In yet another embodiment, m is 0; in others m is 1 or 2.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of formula I. The optically active forms of the compound of the invention may be prepared, for example, by chiral chromatographic separation of a racemate or chemical or enzymatic resolution methodology, by synthesis from optically active starting materials or by asymmetric synthesis based on the procedures described thereafter.

It will also be appreciated by those of skill in the art that certain compounds of the present invention may exist as geometrical isomers, for example E and Z isomers of alkenes. The present invention includes any geometrical isomer of a compound of formula I. It will further be understood that the present invention encompasses tautomers of the compounds of formula I.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:

| Example | Compound | Name |
|---|---|---|
| 4.1 | 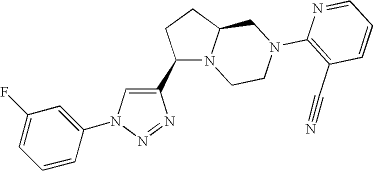 | (±)-2-[(6R,8aS)-6-[1-(3-fluorophenyl)-1H-1,2,3-triazol-4-yl]hexahydropyrrolo[1,2-a]pyrazin-2(1H)-yl]nicotinonitrile |
| 4.2 | 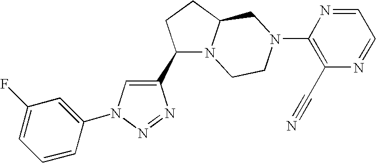 | (±)-3-[(6R,8aS)-6-[1-(3-fluorophenyl)-1H-1,2,3-triazol-4-yl]hexahydropyrrolo[1,2-a]pyrazin-2(1H)-yl]pyrazine-2-carbonitrile |
| 4.3 | 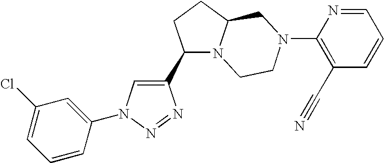 | (±)-2-[(6R,8aS)-6-[1-(3-chlorophenyl)-1H-1,2,3-triazol-4-yl]hexahydropyrrolo[1,2-a]pyrazin-2(1H)-yl]nicotinonitrile |
| 4.4 | 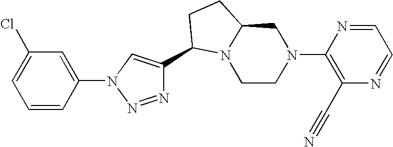 | (±)-3-[(6R,8aS)-6-[1-(3-chlorophenyl)-1H-1,2,3-triazol-4-yl]hexahydropyrrolo[1,2-a]pyrazin-2(1H)-yl]pyrazine-2-carbonitrile |
| 4.5 | 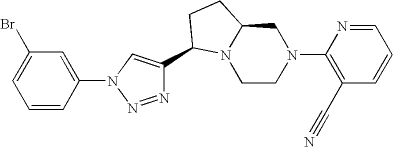 | (±)-2-[(6R,8aS)-6-[1-(3-bromophenyl)-1H-1,2,3-triazol-4-yl]hexahydropyrrolo[1,2-a]pyrazin-2(1H)-yl]nicotinonitrile |
| 4.6 | 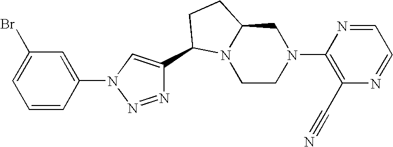 | (±)-3-[(6R,8aS)-6-[1-(3-bromophenyl)-1H-1,2,3-triazol-4-yl]hexahydropyrrolo[1,2-a]pyrazin-2(1H)-yl]pyrazine-2-carbonitrile |
| 4.7 | 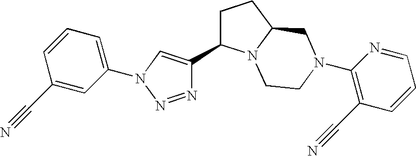 | (±)-2-[(6R,8aS)-6-[1-(3-cyanophenyl)-1H-1,2,3-triazol-4-yl]hexahydropyrrolo[1,2-a]pyrazin-2(1H)-yl]nicotinonitrile |
| 4.8 | 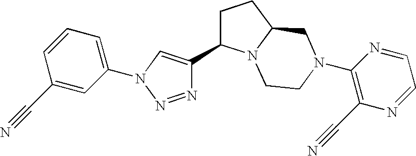 | (±)-3-[(6R,8aS)-6-[1-(3-cyanophenyl)-1H-1,2,3-triazol-4-yl]hexahydropyrrolo[1,2-a]pyrazin-2(1H)-yl]pyrazine-2-carbonitrile |
| 5.1 | 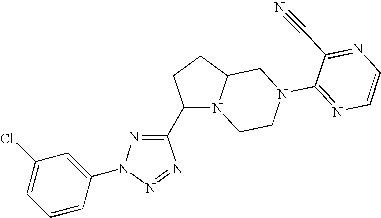 | (±)-3-[(6R,8aS)-6-[2-(3-chlorophenyl)-2H-tetrazol-5-yl]hexahydropyrrolo[1,2-a]pyrazin-2(1H)-yl]pyrazine-2-carbonitrile |
| 5.2 | 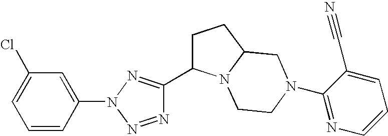 | (±)-2-[(6R,8aS)-6-[2-(3-chlorophenyl)-2H-tetrazol-5-yl]hexahydropyrrolo[1,2-a]pyrazin-2(1H)-yl]nicotinonitrile |
| 5.3 | 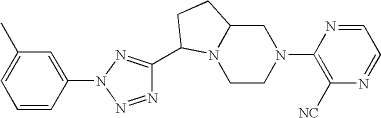 | (±)-3-[(6R,8aS)-6-[2-(3-methylphenyl)-2H-tetrazol-5-yl]hexahydropyrrolo[1,2-a]pyrazin-2(1H)-yl]pyrazine-2-carbonitrile |
| 5.4 | 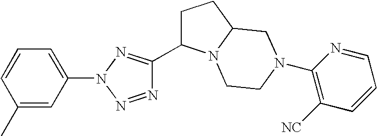 | (±)-2-[(6R,8aS)-6-[2-(3-methylphenyl)-2H-tetrazol-5-yl]hexahydropyrrolo[1,2-a]pyrazin-2(1H)-yl]nicotinonitrile |
| 6.1 | 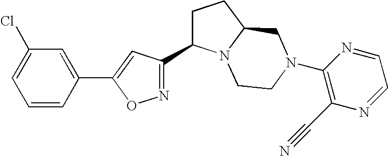 | (±)-3-[(6R,8aS)-6-[5-(3-chlorophenyl)isoxazol-3-yl]hexahydropyrrolo[1,2-a]pyrazin-2(1H)-yl]pyrazine-2-carbonitrile |
| 6.2 | 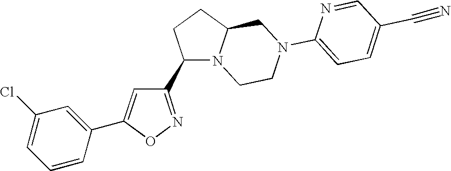 | (±)-6-[(6R,8aS)-6-[5-(3-chlorophenyl)isoxazol-3-yl]hexahydropyrrolo[1,2-a]pyrazin-2(1H)-yl]nicotinonitrile |
| 6.3 | 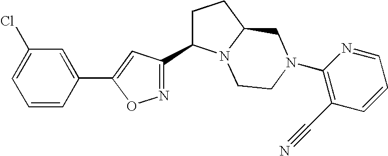 | (±)-2-[(6R,8aS)-6-[5-(3-chlorophenyl)isoxazol-3-yl]hexahydropyrrolo[1,2-a]pyrazin-2(1H)-yl]nicotinonitrile |
| 6.4 | 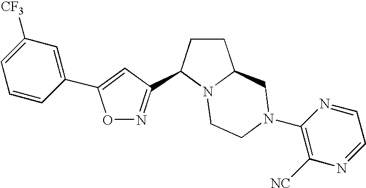 | (±)-3-[(6R,8aS)-6-{5-[3-(trifluoromethyl)phenyl]isoxazol-3-yl}hexahydropyrrolo[1,2-a]pyrazin-2(1H)-yl]pyrazine-2-carbonitrile |
| 6.5 | 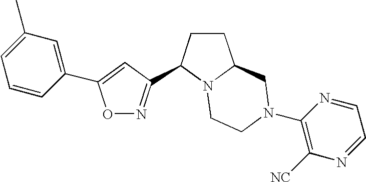 | (±)-3-[(6R,8aS)-6-{5-[3-methylphenyl)isoxazol-3-yl]hexahydropyrrolo[1,2-a]pyrazin-2(1H)-yl]pyrazine-2-carbonitrile |
| 6.6 | 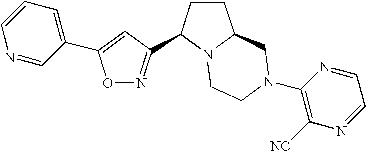 | (±)-3-[(6R,8aS)-6-(5-pyridin-3-ylisoxazol-3-yl)hexahydropyrrolo[1,2-a]pyrazin-2(1H)-yl]pyrazine-2-carbonitrile |
| 6.7 | 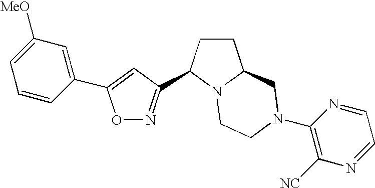 | (±)-3-[(6R,8aS)-6-[5-(3-methoxyphenyl)isoxazol-3-yl]hexahydropyrrolo[1,2-a]pyrazin-2(1H)-yl]pyrazine-2-carbonitrile |
| 6.8 | 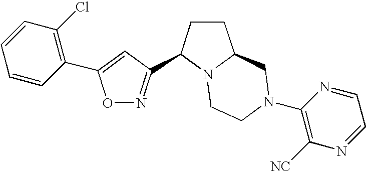 | (±)-3-[(6R,8aS)-6-[5-(2-chlorophenyl)isoxazol-3-yl]hexahydropyrrolo[1,2-a]pyrazin-2(1H)-yl]pyrazine-2-carbonitrile |
| 6.9 | 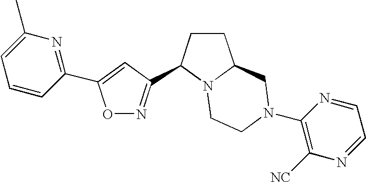 | (±)-3-[(6R,8aS)-6-[5-(6-methylpyridin-2-yl)isoxazol-3-yl]hexahydropyrrolo[1,2-a]pyrazin-2(1H)-yl]pyrazine-2-carbonitrile |
| 6.10 | 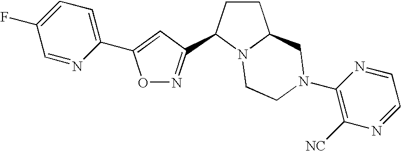 | (±)-3-[(6R,8aS)-6-[5-(5-fluoropyridin-2-yl)isoxazol-3-yl]hexahydropyrrolo[1,2-a]pyrazin-2(1H)-yl]pyrazine-2-carbonitrile |
| 7.1 | 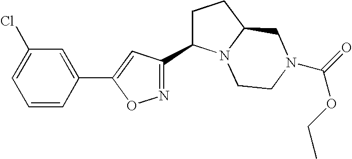 | (±)-Ethyl (6R,8aS)-6-[5-(3-chlorophenyl)isoxazol-3-yl]hexahydropyrrolo[1,2-a]pyrazine-2(1H)-carboxylate |
| 8.1 | 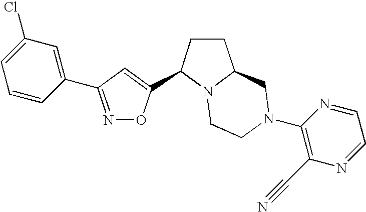 | (±)-3-[(6R,8aS)-6-[3-(3-chlorophenyl)isoxazol-5-yl]hexahydropyrrolo[1,2-a]pyrazin-2(1H)-yl]pyrazine-2-carbonitrile |
| 9.1 | 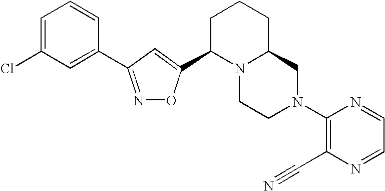 | (±)-6-[(6R,9aS)-6-[5-(3-chlorophenyl)isoxazol-3-yl]octahydro-2H-pyrido[1,2-a]pyrazin-2-yl]pyrazine-2-carbonitrile |
| 9.2 | 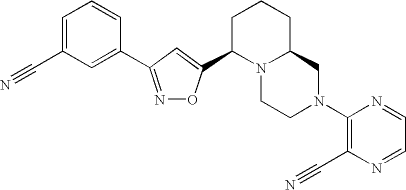 | (±)-6-[(6R,9aS)-6-[5-(3-cyanophenyl)isoxazol-3-yl]octahydro-2H-pyrido[1,2-a]pyrazin-2-yl]pyrazine-2-carbonitrile |
| 9.3 | 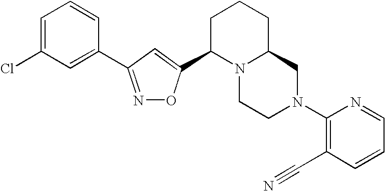 | (±)-6-[(6R,9aS)-6-[3-(3-chlorophenyl)isoxazol-5-yl]octahydro-2H-pyrido[1,2-a]pyrazin-2-yl]nicotinonitrile |
| 9.4 | 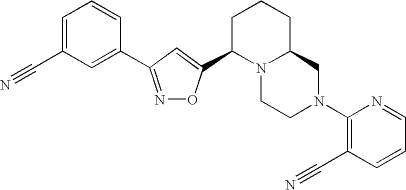 | (±)-2-[(6R,9aS)-6-[3-(3-cyanophenyl)isoxazol-5-yl]octahydro-2H-pyrido[1,2-a]pyrazin-2-yl]nicotinonitrile |
| 10.1 | 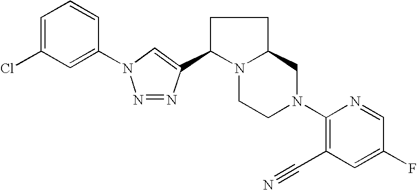 | (±)-2-[(6R,8aS)-6-[1-(3-chlorophenyl)-1H-1,2,3-triazol-4-yl]hexahydropyrrolo[1,2-a]pyrazin-2(1H)-yl]-5-fluoronicotinonitrile |
| 10.2 | 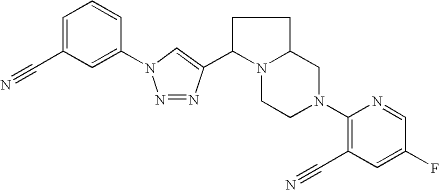 | (±)-6-[(6R,9aS)-6-[5-(3-cyanophenyl)isoxazol-3-yl]hexahydropyrrolo[1,2-a]pyrazin-2-yl]-5-fluoronicotinonitrile |
| 11.1 | 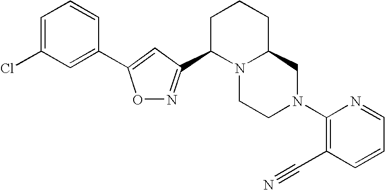 | (±)-2-[(6R,9aS)-6-[5-(3-chlorophenyl)isoxazol-3-yl]octahydro-2H-pyrido[1,2-a]pyrazin-2(1H)-yl]nicotinonitrile |
| 12.1 | 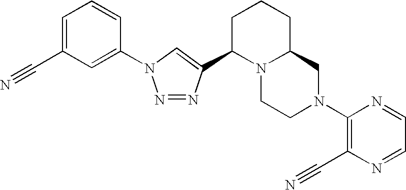 | (±)-6-[(6R,9aS)-6-[5-(3-cyanophenyl)-1,2,3-triazol-4-yl]octahydro-2H-pyrido[1,2-a]pyrazin-2-yl]pyrazine-2-carbonitrile |
| 12.2 | 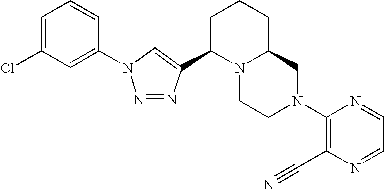 | (±)-6-[(6R,9aS)-6-[5-(3-chloro-phenyl)-1,2,3-triazol-4-yl]octahydro-2H-pyrido[1,2-a]pyrazin-2-yl]pyrazine-2-carbonitrile |
| 12.3 | 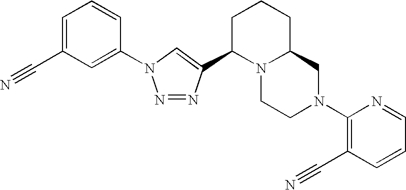 | (±)-6-[(6R,9aS)-6-[5-(3-cyanophenyl)-1,2,3-triazol-4-yl]octahydro-2H-pyrido[1,2-a]pyrazin-2(1H)-yl] nicotinonitrile |
| 12.4 | 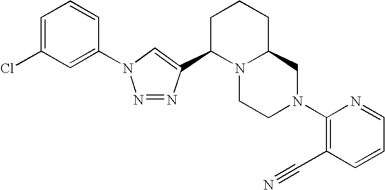 | (±)-6-[(6R,9aS)-6-[5-(3-chloro-phenyl)-1,2,3-triazol-4-yl]octahydro-2H-pyrido[1,2-a]pyrazin-2(1H)-yl] nicotinonitrile |
| 13.1 | 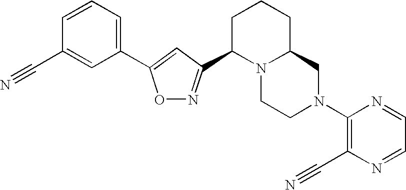 | (±)-3-[(6R,9aS)-6-[5-(3-cyanophenyl)isoxazol-3-yl]octahydro-2H-pyrido[1,2-a]pyrazin-2-yl]pyrazine-2-carbonitrile |
| 13.2 | 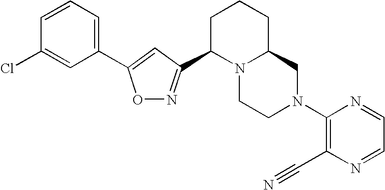 | (±)-3-[(6R,9aS)-6-[5-(3-chlorophenyl)isoxazol-3-yl]octahydro-2H-pyrido[1,2-a]pyrazin-2-yl]pyrazine-2-carbonitrile |
| 13.3 | 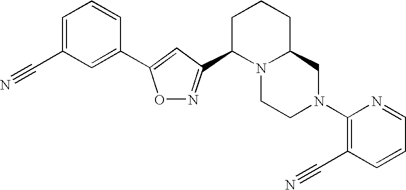 | (±)-2-[(6R,9aS)-6-[5-(3-chlorophenyl)-isoxazol-3-yl]octahydro-2H-pyrido[1,2-a]pyrazin-2-yl]nicotinonitrile |
| 13.4 | 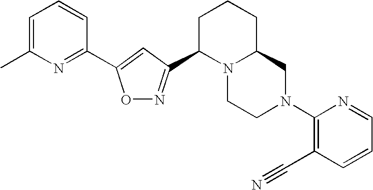 | (±)-2-[(6R,9aS)-6-[5-(6-methylpyrid-2-yl)isoxazol-3-yl]octahydro-2H-pyrido[1,2-a]pyrazin-2-yl]nicotinonitrile |
| 14.1 | 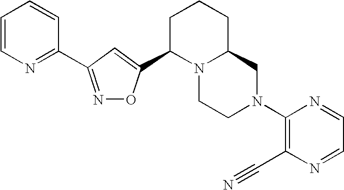 | (±)-6-[(6R,9aS)-6-[5-(pyrid-2-yl)isoxazol-3-yl]octahydro-2H-pyrido[1,2-a]pyrazin-2-yl]pyrazine-2-carbonitrile |
| 15.1 | 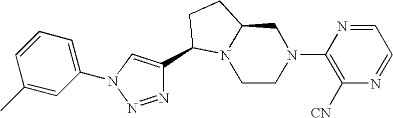 | (±)-3-[(6R,8aS)-6-[1-(3-methylphenyl)-1H-1,2,3-triazol-4-yl]hexahydropyrrolo[1,2-a]pyrazin-2(1H)-yl]pyrazine-2-carbonitrile |
| 15.2 | 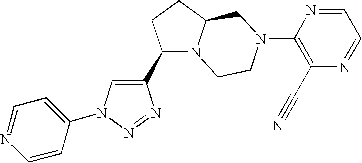 | (±)-3-[(6R,8aS)-6-(1-pyridin-4-yl-1H-1,2,3-triazol-4-yl]hexahydropyrrolo[1,2-a]pyrazin-2(1H)-yl]pyrazine-2-carbonitrile |
| 15.3 | 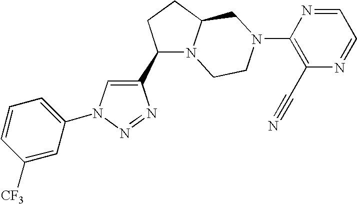 | (±)-3-[(6R,8aS)-6-[1-(3-trifluoromethylphenyl)-1H-1,2,3-triazol-4-yl]hexahydropyrrolo[1,2-a]pyrazin-2(1H)-yl]pyrazine-2-carbonitrile |
| 15.4 | 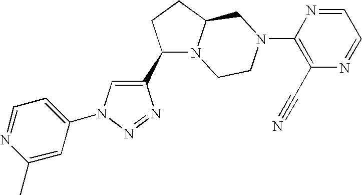 | (±)-3-[(6R,8aS)-6-[1-(2-methylpyridin-4-yl)-1H-1,2,3-triazol-4-yl]hexahydropyrrolo[1,2-a]pyrazin-2(1H)-yl]pyrazine-2-carbonitrile |
| 16.1E1 & E2 | 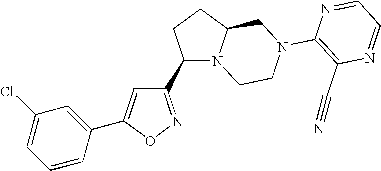 | 3-[(6R,8aS)-6-[5-(3-chlorophenyl)isoxazol-3-yl]hexahydropyrrolo[1,2-a]pyrazin-2(1H)-yl]pyrazine-2-carbonitrile&3-[(6R,8aS)-6-[5-(3-chlorophenyl)isoxazol-3-yl]hexahydropyrrolo[1,2-a]pyrazin-2(1H)-yl]pyrazine-2-carbonitrile |
| 16.2E1 & E2 | 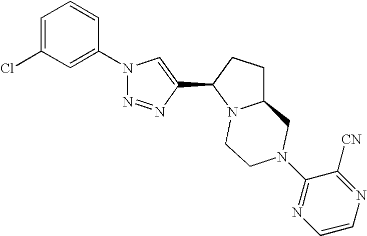 | 3-[(6R,8aS)-6-[1-(3-chlorophenyl)-1H-1,2,3-triazol-4-yl]hexahydropyrrolo[1,2-a]pyrazin-2(1H)-yl] pyrazine-2-carbonitrile&3-[(6S,8aR)-6-[1-(3-chlorophenyl)-1H-1,2,3-triazol-4-yl]hexahydropyrrolo[1,2-a]pyrazin-2(1H)-yl]pyrazine-2-carbonitrile |
| 16.3E1 & E2 | 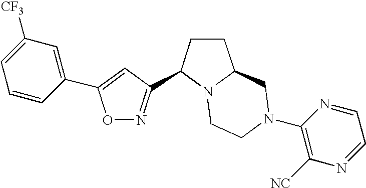 | 3-[(6R,8aS)-6-{5-[3-(trifluoromethyl)phenyl]isoxazol-3-yl}hexahydropyrrolo[1,2-a]pyrazin-2(1H)-yl]pyrazine-2-carbonitrile&3-[(6S,8aR)-6-{5-[3-(trifluoromethyl)phenyl]isoxazol-3-yl}hexahydropyrrolo[1,2-a]pyrazin-2(1H)-yl]pyrazine-2-carbonitrile |
| 16.4E1 & E2 | 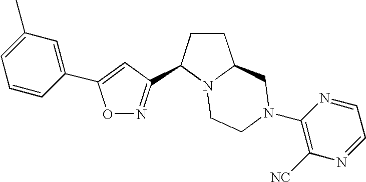 | 3-[(6R,8aS)-6-[5-(3-methylphenyl)isoxazol-3-yl]hexahydropyrrolo[1,2-a]pyrazin-2(1H)-yl]pyrazine-2-carbonitrile&3-[(6S,8aR)-6-[5-(3-methylphenyl)isoxazol-3-yl]hexahydropyrrolo[1,2-a]pyrazin-2(1H)-yl]pyrazine-2-carbonitrile |
| 16.5E1 & E2 | 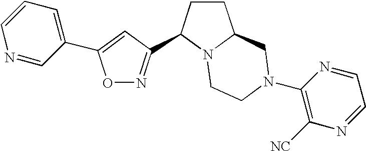 | 3-[(6R,8aS)-6-(5-pyridin-3-ylisoxazol-3-yl)hexahydropyrrolo[1,2-a]pyrazin-2(1H)-yl]pyrazine-2-carbonitrile&3-[(6S,8aR)-6-(5-pyridin-3-ylisoxazol-3-yl)hexahydropyrrolo[1,2-a]pyrazin-2(1H)-yl]pyrazine-2-carbonitrile |
| 16.6E1 & E2 | 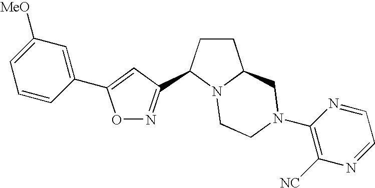 | 3-[(6R,8aS)-6-[5-(3-methoxyphenyl)isoxazol-3-yl]hexahydropyrrolo[1,2-a]pyrazin-2(1H)-yl]pyrazine-2-carbonitrile&3-[(6S,8aR)-6-[5-(3-methoxyphenyl)isoxazol-3-yl]hexahydropyrrolo[1,2-a]pyrazin-2(1H)-yl]pyrazine-2-carbonitrile |
| 16.7E1 & E2 | 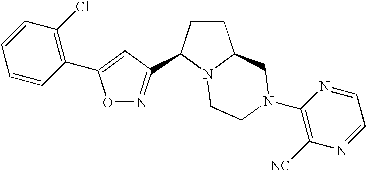 | 3-[(6R,8aS)-6-[5-(2-chlorophenyl)isoxazol-3-yl]hexahydropyrrolo[1,2-a]pyrazin-2(1H)-yl]pyrazine-2-carbonitrile&3-[(6S,8aR)-6-[5-(2-chlorophenyl)isoxazol-3-yl]hexahydropyrrolo[1,2-a]pyrazin-2(1H)-yl]pyrazine-2-carbonitrile |
| 16.8E1 & E2 | 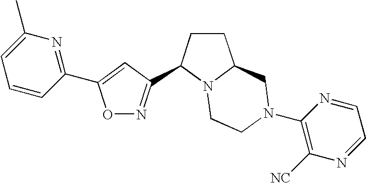 | 3-[(6R,8aS)-6-[5-(6-methylpyridin-2-yl)isoxazol-3-yl]hexahydropyrrolo[1,2-a]pyrazin-2(1H)-yl]pyrazine-2-carbonitrile&3-[(6S,8aR)-6-[5-(6-methylpyridin-2-yl)isoxazol-3-yl]hexahydropyrrolo[1,2-a]pyrazin-2(1H)-yl]pyrazine-2-carbonitrile |
| 16.9E1 & E2 | 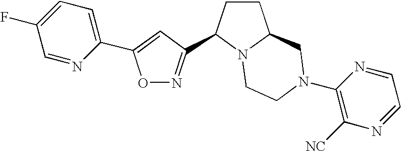 | 3-[(6R,8aS)-6-[5-(5-fluoropyridin-2-yl)isoxazol-3-yl]hexahydropyrrolo[1,2-a]pyrazin-2(1H)-yl]pyrazine-2-carbonitrile&3-[(6S,8aR)-6-[5-(5-fluoropyridin-2-yl)isoxazol-3-yl]hexahydropyrrolo[1,2-a]pyrazin-2(1H)-yl]pyrazine-2-carbonitrile |
| 16.10E1 & E2 | 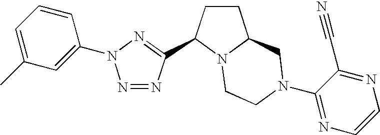 | 3-[(6R,8aS)-6-[2-(3-methylphenyl)-2H-tetrazol-5-yl]hexahydropyrrolo[1,2-a]pyrazin-2(1H)-yl]pyrazine-2-carbonitrile&3-[(6S,8aR)-6-[2-(3-methylphenyl)-2H-tetrazol-5-yl]hexahydropyrrolo[1,2-a]pyrazin-2(1H)-yl]pyrazine-2-carbonitrile |
| 16.11E1 & E2 | 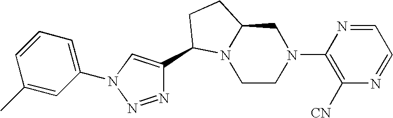 | 3-[(6R,8aS)-6-[1-(3-methylphenyl)-1H-1,2,3-triazol-4-yl]hexahydropyrrolo[1,2-a]pyrazin-2(1H)-yl]pyrazine-2-carbonitrile&3-[(6S,8aR)-6-[1-(3-methylphenyl)-1H-1,2,3-triazol-4-yl]hexahydropyrrolo[1,2-a]pyrazin-2(1H)-yl]pyrazine-2-carbonitrile |
| 16.12E1 & E2 | 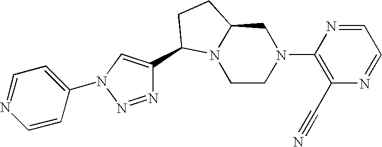 | 3-[(6R,8aS)-6-(1-pyridin-4-yl-1H-1,2,3-triazol-4-yl)hexahydropyrrolo[1,2-a]pyrazin-2(1H)-yl]pyrazine-2-carbonitrile&3-[(6S,8aR)-6-(1-pyridin-4-yl-1H-1,2,3-triazol-4-yl)hexahydropyrrolo[1,2-a]pyrazin-2(1H)-yl]pyrazine-2-carbonitrile |
| 16.14E1 & E2 | 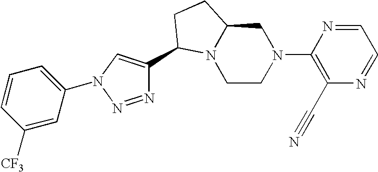 | 3-[(6R,8aS)-6-[1-(3-trifluoromethylphenyl)-1H-1,2,3-triazol-4-yl]hexahydropyrrolo[1,2-a]pyrazin-2(1H)-yl]pyrazine-2-carbonitrile&3-[6S,8aR)-6-[1-(3-trifluoromethylphenyl)-1H-1,2,3-triazol-4-yl]hexahydropyrrolo[1,2-a]pyrazin-2(1H)-yl]pyrazine-2-carbonitrile |
| 16.15E1 & E2 | 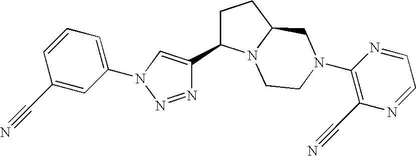 | 3-[(6R,8aS)-6-[1-(3-cyanophenyl)-1H-1,2,3-triazol-4-yl]hexahydropyrrolo[1,2-a]pyrazin-2(1H)-yl]pyrazine-2-carbonitrile&3-[(6S,8aR)-6-[1-(3-cyanophenyl)-1H-1,2,3-triazol-4-yl]hexahydropyrrolo[1,2-a]pyrazin-2(1H)-yl]pyrazine-2-carbonitrile |
| 16.16E1 & E2 | 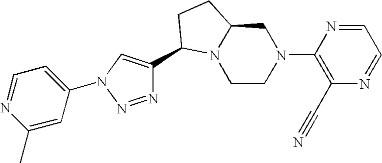 | 3-[(6R,8aS)-6-[1-(2-methylpyridin-4-yl)-1H-1,2,3-triazol-4-yl]hexahydropyrrolo[1,2-a]pyrazin-2(1H)-yl]pyrazine-2-carbonitrile&3-[6S,8aR)-6-[1-(2-methylpyridin-4-yl)-1H-1,2,3-triazol-4-yl]hexahydropyrrolo[1,2-a]pyrazin-2(1H)-yl]pyrazine-2-carbonitrile |

### Pharmaceutical Composition

The compounds of the present invention may be formulated into conventional pharmaceutical composition comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

It has been found that the compounds according to the present invention, exhibit a high degree of potency and selectivity for individual metabotropic glutamate receptor (mGluR) subtypes. Accordingly, the compounds of the present invention are expected to be useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of Formula I, as defined hereinbefore, for use in therapy.

The invention relates to compounds of Formula I, as defined hereinbefore, for use in treatment of mGluR5-mediated disorders.

The invention relates to compounds of Formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of Formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including cancer, angina, renal or biliary colic, menstruation, migraine and gout.

The invention relates to compounds of Formula I as defined hereinbefore, for use in treatment of stroke, head trauma, anoxic and ischemic injuries, hypoglycemia, cardiovascular diseases and epilepsy.

The present invention relates also to the use of a compound of Formula I as defined hereinbefore, in the manufacture of a medicament for the treatment of mGluR Group I receptor-mediated disorders and any disorder listed above.

One embodiment of the invention relates to the use of a compound according to Formula I in the treatment of gastrointestinal disorders.

Another embodiment of the invention relates to the use of a Formula I compound for the manufacture of a medicament for inhibition of transient lower esophageal sphincter relaxations, for the treatment of GERD, for the prevention of G.I. reflux, for the treatment regurgitation, for treatment of asthma, for treatment of laryngitis, for treatment of lung disease, for the management of failure to thrive, for the treatment of irritable bowel disease (IBS) and for the treatment of functional dyspepsia (FD).

The invention also provides a method of treatment of mGluR5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

### Non-Medical Use

In addition to their use in therapeutic medicine, the compounds of Formula I, as well as salts and hydrates of such compounds, are useful as pharmacological tools in the development and standardization of in vitro and in vivo test systems for the evaluation of the effects of inhibitors of mGluR related activity in laboratory animals such as cats, dogs, rabbits, monkeys, rats and mice, as part of the search for new therapeutics agents.

### Process of Preparation

Another aspect of the present invention provides processes for preparing compounds of Formula I, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It also is to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, normal and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

The bicyclic intermediates may be prepared in a number of ways. For example, as shown in Scheme 1, pyrrolo-pyrazines b are accessible from the meso-dibromide a in a single step. Subsequent functional group manipulations lead to a diverse group of bicyclic piperazines containing aldehyde (e) and acetylenic (f) moieties which may be converted to a number of heterocyclic products.

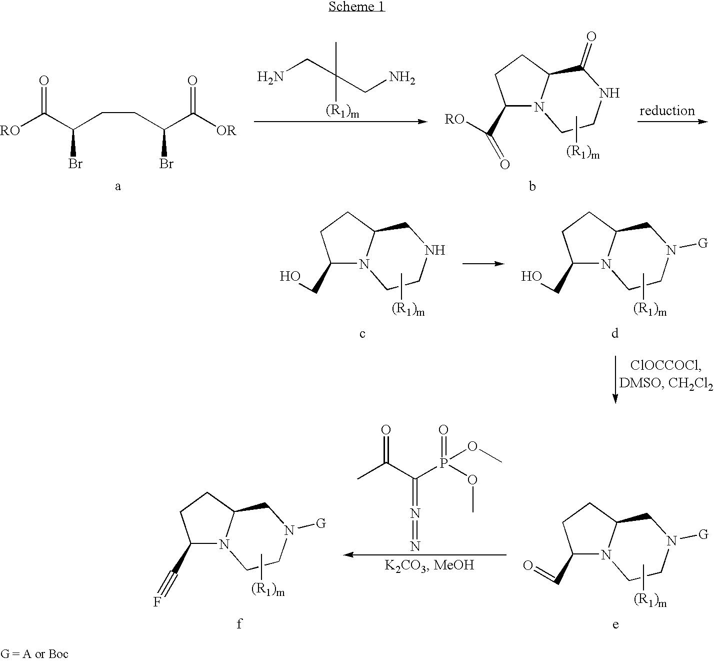

The analogous ring expanded piperidino-piperazinyl alcohol k may be prepared, as shown in Scheme 2, from reduction of pyridyl diester g to the piperidine diester h, acylation and ring closure to the diketopiperazine j, followed by simultaneous reduction of the ester and amide moieties. Arylation or protection, conversion to the analogous aldehyde l and acetylene m may be carried out under the same conditions.

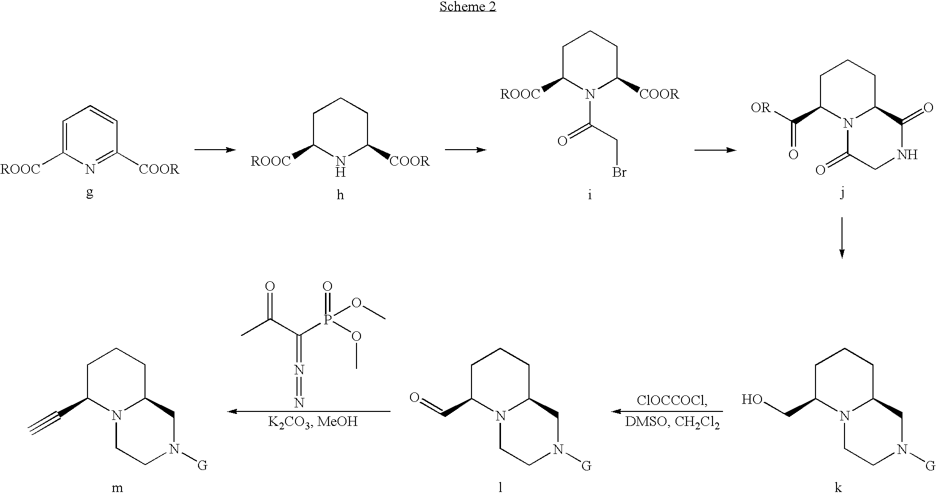

Compounds n in which Hy is a 1,2,3-triazole may be prepared by the method shown in Scheme 3, below, by treatment of the above bicyclic acetylene f with an aryl iodide in the presence of sodium azide and a copper catalyst, according to the procedure of Organic Letters 2004, Vol. 6, No. 22, 3897-3899. Alternatively, the triazole may be formed using an isolated aryl azide generated from an aniline via diazotization and trapping with sodium azide (as described in WO05/080379).

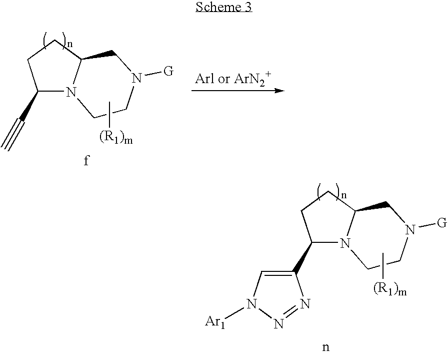

Acetylene f may also be used to prepare compounds o, in which Hy is an isoxazole, as shown in Scheme 4, below, by treatment with an aryl chloroimidate, which is readily available from the corresponding oxime by treatment with NCS.

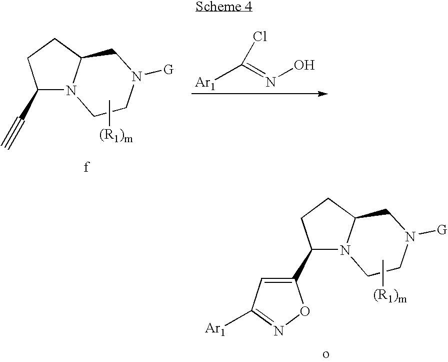

The isomeric isoxazole r may be prepared as shown in Scheme 5, below, from the aldehyde e via the bicyclic chloroimidate p using an aryl acetylene (the basic amine is protected by the addition of an acid to form the salt, for example HCl). Alternatively, the propargylic alcohol q may be prepared by addition of an acetylide anion to aldehyde e, followed by oxidation to the ketone using mild conditions (such as Swern oxidation), followed by formation of the oxime and cyclization to the isoxazole r. Since the unsaturated ketone and oxime intermediates are unstable, these are typically used immediately following preparation without chromatographic purification.

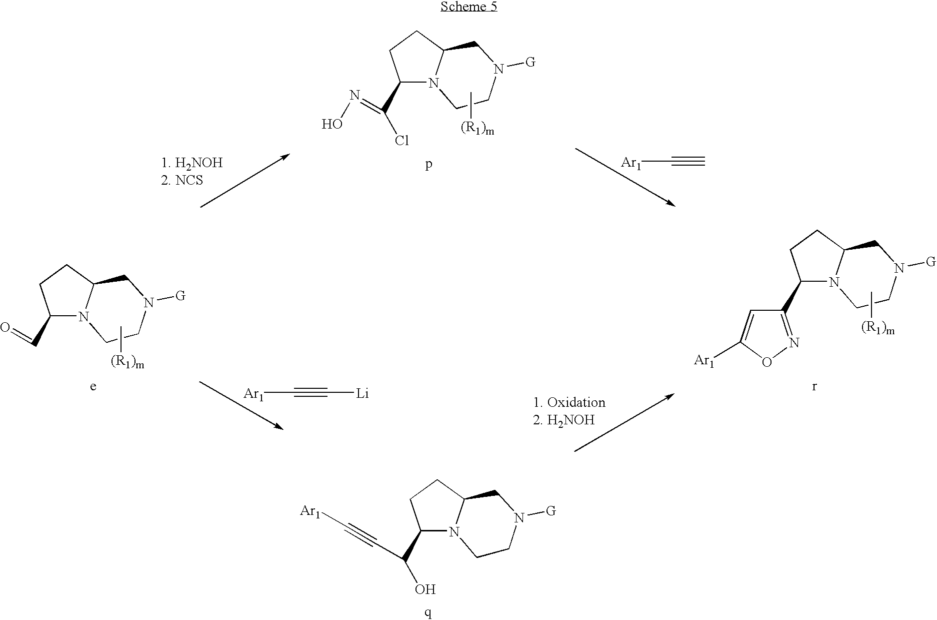

Compounds y, in which Hy is a tetrazole, may be prepared by the methods shown in Scheme 6, below, treating an amine such as benzyl amine with the meso-dibromide a to provide the pyrrolidine ester s. Removal of the benzyl group and subsequent protection as a carbamate (such as tetrabutyloxycarbonyl), followed by monohydrolysis of the diester, facilitates preparation of the pyrrolidine acid s. The acid may be converted, via the nitrile t, to the tetrazole u, which may be subsequently arylated using an iodonium reagent in the presence of palladium catalyst (for example Pd₂ dba₃) and a ligand such as BINAP in the presence of a base such as NaOtBu, as described in published PCT application WO05080386. After removal of the protecting group the aryl tetrazole pyrrolidine v may be converted to the bicyclic intermediate via, for example, the diketopiperazine x in 2 steps, using an acylating agent such as bromoacetyl chloride followed by cyclization with ammonia. Reduction and introduction of group A provides the intermediate tetrazole compound y.

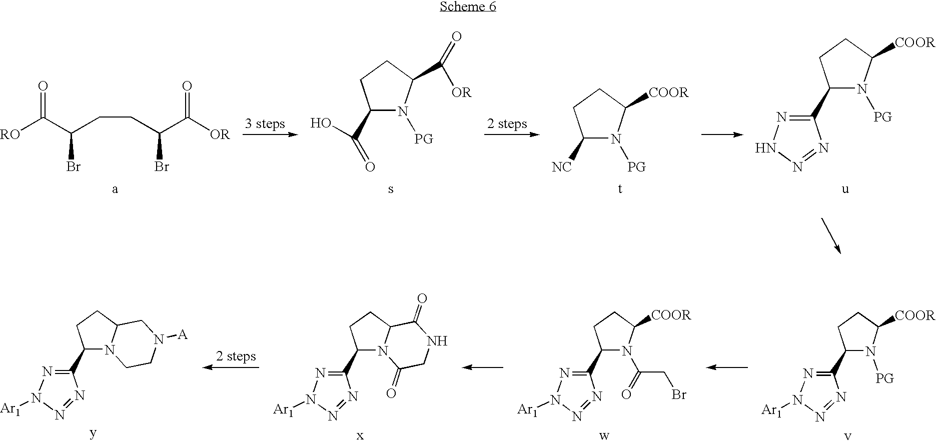

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### General Methods

All starting materials are commercially available or earlier described in the literature.

The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet). Unless otherwise indicated, in the tables below ¹H NMR data was obtained at 300 MHz, using CDCl₃ as the solvent.

Purification of products were also done using Chem Elut Extraction Columns (Varian, cat #1219-8002), Mega BE-SI (Bond Elut Silica) SPE Columns (Varian, cat #12256018; 12256026; 12256034), or by flash chromatography in silica-filled glass columns.

Microwave heating was performed in an Emrys Optimizer from Biotage/Personal Chemistry or a Smith Synthesizer Single-mode microwave cavity producing continuous irradiation at 2450 MHz (Personal Chemistry AB, Uppsala, Sweden).

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

Intracellular calcium mobilization was measured by detecting changes in fluorescence of cells loaded with the fluorescent indicator fluo-3. Fluorescent signals were measured using the FLIPR system (Molecular Devices). A two addition experiment was used that could detect compounds that either activate or antagonize the receptor.

For FLIPR analysis, cells expressing human mGluR5d were seeded on collagen coated clear bottom 96-well plates with black sides and analysis of [Ca²⁺]ᵢ mobilization was done 24 hours after seeding.

FLIPR experiments were done using a laser setting of 0.800 W and a 0.4 second CCD camera shutter speed. Each FLIPR experiment was initiated with 160 μL of buffer present in each well of the cell plate. After each addition of the compound, the fluorescence signal was sampled 50 times at 1 second intervals followed by 3 samples at 5 second intervals. Responses were measured as the peak height of the response within the sample period.

EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 minutes with 10 mM LiCl. Compounds (agonists) were added and incubated for 30 minutes at 37° C. Antagonist activity was determined by pre-incubating test compounds for 15 minutes, then incubating in the presence of glutamate (80 μM) or DHPG (30 μM) for 30 minutes. Reactions were terminated by the addition of perchloric acid (5%). Samples were collected and neutralized, and inositol phosphates were separated using Gravity-Fed Ion-Exchange Columns.

A detailed protocol for testing the compounds of the invention is provided below in Pharmaceutical Examples.

### Abbreviations

BOC tert-butoxycarbonyl

### BSA Bovine Serum Albumin

### CCD Charge Coupled Device

### CRC Concentration Response Curve

DBU 1,8-diazabicyclo[5.4.0]undec-7-ene
DCM dichloromethane
DHPG 3,5-dihydroxyphenylglycine;
DIBAL diisobutylaluminum hydride

### DMF N,N-dimethylformamide

DMSO dimethyl sulfoxide

### EDTA Ethylene Diamine Tetraacetic Acid

Et₃N triethylamine
EtOH ethanol
FLIPR Fluorometric Imaging Plate reader
GC/MS gas chromatograph coupled mass spectroscopy
GHEK Human Embryonic Kidney expressing Glutamate Transporter
HEPES 4-(2-hydroxyethyl)-1-piperazineethanesulfonic acid (buffer)
IP₃ inositol triphosphate
MCPBA 3-chloroperbenzoic acid
MeOH methanol

### NMP N-Methylpyrrolidinone

NMR nuclear magnetic resonance
PCC pyridinium chlorochromate
ppm parts per million
RT room temperature
SPE solid phase extraction
TFA trifluoroacetic acid
THF tetrahydrofuran
TLC thin layer chromatography

### Example 1.1

### (±)-(6R,8aS)-6-[1-(3-fluorophenyl)-1H-1,2,3-triazol-4-yl]octahydropyrrolo[1,2-a]pyrazine

### (i) (±)-Ethyl (6R,8aS)-1-oxooctahydropyrrolo[1,2-a]pyrazine-6-carboxylate

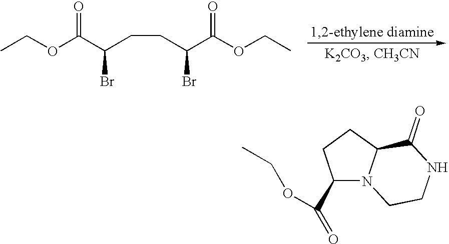

To a mixture of 1,2-ethylene diamine (20 mL, 0.28 mol), K₂CO₃ (40 g, 0.29 mol) and CH₃CN (300 mL) was added slowly a solution of diethyl meso-2,5-dibromoadipate (50 g, 0.14 mol) in CH₃CN (200 mL) over 36 h at room temperature. The solvent was removed and DCM (300 mL) was added. After filtration, the DCM was evaporated to afford the crude product (32 g, purity >90%). ¹H NMR (300 MHz, CDCl₃): δ (ppm) 1.30 (t, 3H), 1.96-2.18 (m, 4H), 2.52 (m, 1H), 2.94 (m, 1H), 3.15 (m, 1H), 3.35 (m, 2H), 3.60 (m, 1H), 4.23 (q, 2H), 6.12 broad, 1H).

### (ii) (±)-(6R,8aS)-Octahydropyrrolo[1,2-a]pyrazin-6-ylmethanol

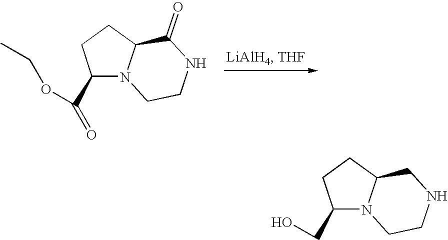

To a suspension of LiAlH₄ (16 g, 0.42 mol) in THF (350 mL) was added a solution of (±)-ethyl (6R,8aS)-1-oxooctahydropyrrolo[1,2-a]pyrazine-6-carboxylate (32 g, 0.15 mol) in THF (150 mL) at 0° C. over 30 min. The reaction mixture was stirred at room temperature overnight and at 80° C. for 2 h. To the resulting mixture was added carefully NaOH aq (10% 18 mL) over 30 min at 0° C. After stirring for additional 30 min, the mixture was filtered through Celite® and the filtrate was concentrated to afford the crude aminoalcohol (20.5 g, purity >85%). ¹H NMR (300 MHz, CDCl₃): δ (ppm) 1.28 (m, 1H), 1.76-1.87 (m, 3H), 2.15 (m, 2H), 2.45 (m, 2H), 2.76 (m, 1H), 3.01 (m, 2H), 3.13 (m, 1H), 3.46 (broad d, 1H), 3.70-3.79 (m, 2H).

### (iii) (±)-Tert-Butyl (6R,8aS)-6-(hydroxymethyl)hexahydropyrrolo[1,2-a]pyrazine-2(1H)-carboxylate

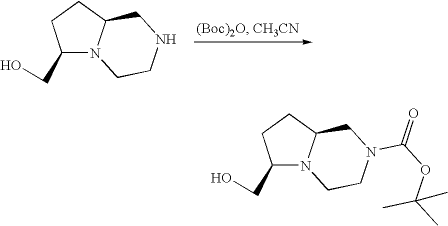

To a solution of (±)-(6R,8aS)-octahydropyrrolo[1,2-a]pyrazin-6-ylmethanol (9 g, crude) in CH₃CN (120 mL) was added (Boc)₂O (13.5 g, 62 mmol) at 0° C. over 10 min. The mixture was stirred at room temperature for 2 h. To the resulting mixture was added Na₂CO₃ aq (sat. 200 mL) and extracted with ethyl acetate (180 mL×3). The combined extract was dried and the solvent was removed with rotary evaporator to give residue which was purified on silica gel column to afford boc protected alcohol (8 g, 64%). ¹H NMR (300 MHz, CDCl₃): δ (ppm) 1.28 (m, 1H), 1.48 (s, 9H), 1.79 (m, 3H), 2.06 (m, 2H), 2.53 (m, 2H), 2.65-3.00 (m, 2H), 3.58 (m, 1H), 4.13 (m, 1H), 4.12 (broad, 2H).

### (iv) (±)-Tert-butyl (6R,8aS)-6-formylhexahydropyrrolo[1,2-a]pyrazine-2(1H)-carboxylate

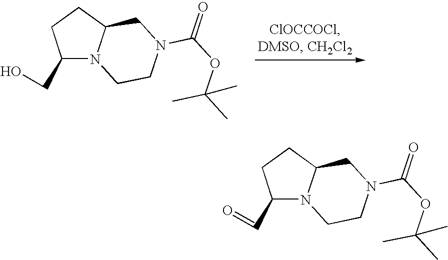

To a solution of oxalyl chloride (2M, 3.3 mL, 6.6 mmol) in DCM (12 mL) was added DMSO (0.71 mL, 10 mmol) at −78° C. After stirring 10 min a solution of (±)-tert-butyl (6R,9aS)-6-(hydroxymethyl)octahydro-2H-pyrido[1,2-a]pyrazine-2-carboxylate (850 mg, 3.3 mmol) in DCM (6 mL) was added. The reaction mixture was stirred at −78° C. for 1 h. Et₃N (2 mL) was added and the resulting mixture was stirred at room temperature for 30 min, then poured into DCM (30 mL)/NH₃—H₂O (10%, 10 mL).

The organic phase was separated, dried over Na₂SO₄ and concentrated to give crude aldehyde.

### v) (±)-Tert-butyl (6R,8aS)-6-ethynylhexahydropyrrolo[1,2-a]pyrazine-2(1H)-carboxylate

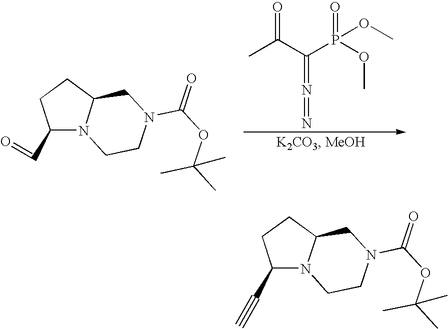

To the crude aldehyde was added MeOH (30 mL), K₂CO₃ and dimethyl (1-diazo-2-oxopropyl)phosphonate (768 mg, 4.0 mmol) at room temperature. After stirring at room temperature for 50 min, the resulting mixture was concentrated. The residue was dissolved with ethyl acetate and filtered. After removal of the solvent, flash chromatography on silica gel afforded pure acetylene (557 mg, 64%). ¹H NMR 300 MHz, (CDCl₃) δ (ppm) 1.48 (s, 9H), 1.55 (m, 1H), 1.66-2.2 (m, 5H), 2.34 (s, 1H), 2.63 (broad, 1H), 2.90 (broad t, 2H), 3.25 (broad d, 1H), 4.16 (broad, 2H).

### (vi) (±)-(6R,8aS)-6-[1-(3-fluorophenyl)-1H-1,2,3-triazol-4-yl]octahydropyrrolo[1,2-a]pyrazine

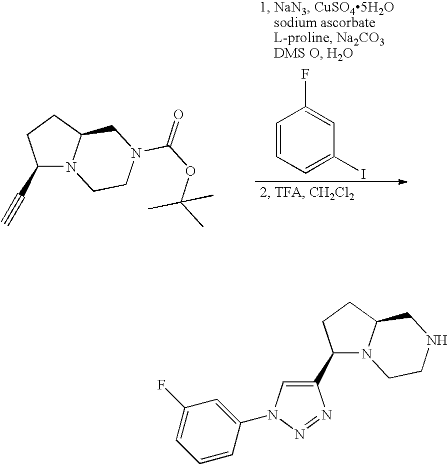

A mixture of (±)-tert-butyl (6R,8aS)-6-ethynylhexahydropyrrolo[1,2-a]pyrazine-2(1H)-carboxylate (256 mg, 1.0 mmol), 3-fluoroiodobenzene (266 mg, 1.2 mmol), NaN₃ (80 mg, 1.2 mmol), CuSO₄5H₂O (26 mg, 0.05 mmol), sodium ascorbate (40 mg, 0.1 mmol), L-proline (24 mg, 0.2 mmol), Na₂CO₃ (22 mg, 0.2 mmol), DMSO (1.8 mL) and H₂O (0.2 mL) was stirred at 68° C. for 8 h. The resulting mixture was diluted with ethyl acetate and washed with saturated Na₂CO_{3(aq)}. The organic phase was concentrated and subjected to silica gel column to afford boc-protected triazole. TFA (1 mL) was added to the triazole in DCM (2 mL) at 0° C. The resulting mixture was stirred at 0° C. for 30 min and then at room temperature for 90 min. DCM and excess TFA were removed in vacuo. DCM was added and the solution was washed with saturated Na₂CO₃ aq, concentrated and dried under vacuum pump to give amine (218 mg, 71%). ¹H NMR 300 MHz, (CDCl₃) δ (ppm) 1.58 (m, 1H), 1.85-2.35 (m, 5H), 2.61 (dd, 1H), 2.66-3.16 (m, 4H), 3.63 (dd, 1H), 7.14 (m, 1H), 7.52 (m, 3H), 7.93 (s, 1H).

The following compounds were synthesized in a similar manner:

| Example | Structure | Name | Yield |
|---|---|---|---|
| 1.2 | 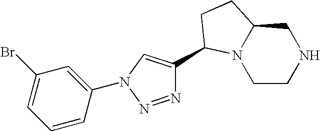 | (±)-(6R, 8aS)-6-[1-(3-bromophenyl)-1H-1,2,3-triazol-4-yl] octahydro-pyrrolo[1,2-a]pyrazine | 79% |
| NMR | 1.58 (m, 1 H), 1.83-2.33 (m, 5 H), 2.61 (dd, 1 H), 2.7-3.14 (m, 4 H), 3.62 (dd, 1 H), 7.42(dd, 1 H), 7.55 (dd, 1 H), 7.70 (dd, 1 H), 7.92 (s, 1 H), 7.94 (dd, 1 H). | | |
| 1.3 | 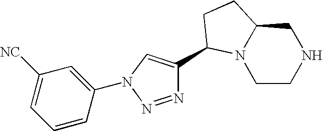 | (±)-3-{4-[(6R, 8aS)-octahydro-pyrrolo[1,2-a]pyrazin-6-yl]-1H-1,2,3-triazol-1-yl}benzonitrile | 79% |
| NMR | 1.90 (m, 1 H), 2.23-2.55 (m, 5 H), 3.05-3.73 (m, 6 H), 4.2 (broad, 1 H), 7.74 (m, 2 H), 8.02(dd, 1 H), 8.09 (s, 1 H), 8.3 (s, 1 H). | | |
| 1.4 | 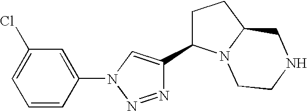 | (±)-(6R, 8aS)-6-[1-(3-chlorophenyl)-1H-1,2,3-triazol-4-yl] octahydro-pyrrolo[1,2-a]pyrazine | 79% |

| | |
|---|---|
| NMR | 1.62 (m, 1 H), 1.85-2.30 (m, 5 H), 2.60 (dd, 1 H), 2.7-3.16 (m, 4 H), 3.62 (dd, 1 H), 7.42(m, 2 H), 7.65 (dd, 1 H), 7.80 (dd, 1 H), 7.92 (s, 1 H). |

### Example 2

### (±)-6-[2-(3-chlorophenyl)-2H-tetrazol-5-yl]octahydropyrrolo[1,2-a]pyrazine

### (i) Copper(II) 2-phenylcyclopropanecarboxyate

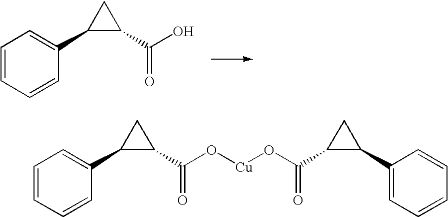

Sodium hydroxide (0.81 g, 20.25 mmol) in water (10 mL) was added to 2-phenylcyclopropanecarboxyate (32.4 g, 20.0 mmol) and the mixture was stirred until the solid completely dissolved. A solution of copper(II) sulfate (2.44 g, 10.0 mmol) in water was added in a dropwise manner. The mixture was stirred for 2 h, and the pale blue precipitate was collected by filtration, dried under vacuum and used without further purification.

### ii) Bis(acetyloxy)(3-chlorophenyl)-λ-3-iodane

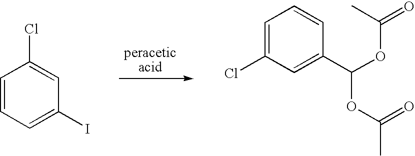

Peracetic acid (65.7 mL, 40%) was added over 30 min to 3-chloro-1-iodobenzene (21.0 mL, 169.6 mmol). The mixture was stirred at 30° C. for 1.5 h and then cooled at 4° C. overnight prior to the addition of acetic acid in water (10%, 50 mL). Filtration and rinsing sequentially with acetic acid in water (10%, 2×25 mL) and ether (2×50 mL) yielded the title compound (58.17 g, 96%, white crystals). ¹H NMR (300 MHz, CDCl₃): δ (ppm) 8.1 (t, 1H), 7.99 (dm, 1H), 7.57 (dm, 1H), 7.46 (t, 1H), 2.04 (s, 6H)

In a similar manner the following compound was synthesized (using 32% peracetic acid, and washed sequentially with ice water and ether):

| example | Structure | Name | Yield |
|---|---|---|---|
| 2.2 ii | 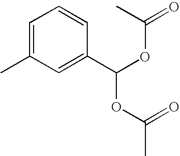 | bis(acetyloxy)(3-methylphenyl)-λ-3-iodane | 80% |

| | |
|---|---|
| NMR | 7.92 (m, 2 H), 7.39 (m, 2 H), 2.43 (s, 3 H), 2.01 (s, 6 H) |
| 400 MHz | |

### iii) Bis-(3-chloro-phenyl)-iodonium tetrafluoroborate

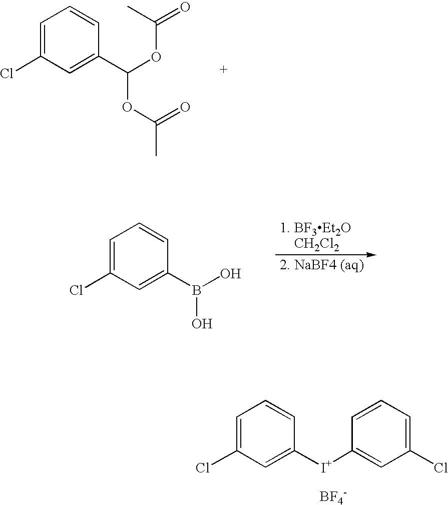

To stirred mixture of 3-chlorophenylboronic acid 0.821 g (5.25 mmol) and BF₃.Et₂O (0.78 g, 5.5 mmol) in DCM (50 mL) at 0° C. was added a solution of bis(acetyloxy)(3-chlorophenyl)-λ-3-iodane (1.78 g, 5 mmol) in DCM (50 mL) under argon, and the reaction mixture was stirred for 1.5 h at 0° C. Saturated aqueous NH₄BF₄ (10.5 g, 100 mol) was added and the reaction mixture was stirred for 1 h, poured into water and extracted with DCM. The organic layer was concentrated to give a solid residue, which was triturated with diethyl ether to give the title compound (off-white solid, 1.70 g, 78%). ¹H NMR (300 MHz, CDCl₃): δ (ppm) 8.02 (m, 4H), 7.58 (dm, 2H), 7.4 (t, 2H).

In a similar manner the following compound was synthesized:

| example | Structure | Name | Yield |
|---|---|---|---|
| 2.2 iii | 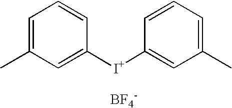 | bis(3-methylphenyl)iodoniumtetrafluoroborate | 81% |

| | |
|---|---|
| NMR | 7.90 (m, 4 H), 7.40 (m, 4 H), 2.42 (s, 6 H) |
| 400 MHz | |

### iv) (±)-diethyl (2R,5S)-1-benzylpyrrolidine-2,5-dicarboxylate

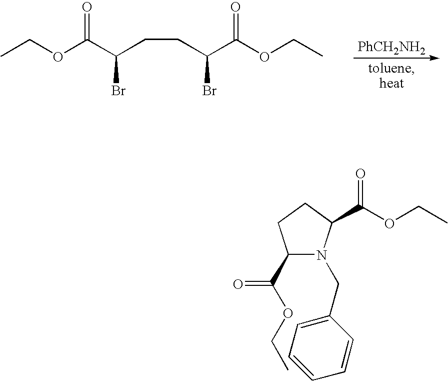

Benzyl amine (6 mL, 54 mmol) was added a solution of diethyl meso-2,5-dibromoadipate (6.5 g, 18 mmol) in toluene (100 mL) at 68° C. and the mixture was heated for 3 days. After cooling to room temperature, the product was partitioned between ethyl acetate and saturated aqueous Na₂CO₃. The organic layer was dried and concentrated in vacuo. Flash chromatography (silica) afforded the title compound (4.5 g, 82%).

### v) (±)-diethyl (2R,5S)-pyrrolidine-2,5-dicarboxylate

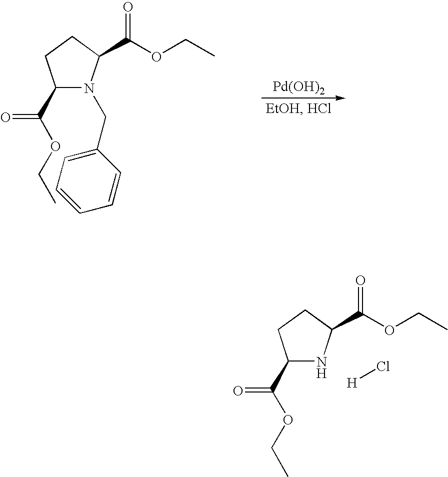

A mixture of (±)-diethyl (2R,5S)-1-benzylpyrrolidine-2,5-dicarboxylate (4.5 g, 14.7 mmol) and Pd(OH)₂ on carbon (300 mg) in EtOH (80 mL) and HCl (20 mL, 1M aqueous) was stirred under an atmosphere of H₂(g) at room temperature overnight. After filtration of the catalyst, the EtOH was removed in vacuo, and the product was partitioned between ethyl acetate and saturated aqueous Na₂CO₃. The organic layer was dried and concentrated in vacuo to afford the title compound (2.8 g, 88%). ¹H NMR (300 MHz, CDCl₃): δ (ppm) 4.59 (t, 2H), 4.33 (q, 4H), 2.51 (m, 2H), 2.24 (m, 2H), 1.35 (t, 6H).

### vi) (±)-t-tert-butyl 2,5-diethyl (2R,5S)-pyrrolidine-1,2,5-tricarboxylate

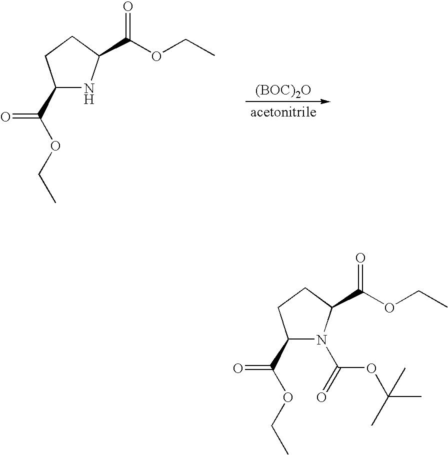

(BOC)₂O (4.36 g, 20.0 mmol) was added a solution of (±)-diethyl (2R,5S)-pyrrolidine-2,5-dicarboxylate (2.8 g, 13.0 mmol) in CH₃CN (30 mL) at 0° C. The mixture was stirred at room temperature for 3 days, and then the solvent was concentrated in vacuo. Flash chromatography (silica) afforded the title compound (3.88 g, 95%).

### (vii) (±)-(2R,5S)-1-(tert-butoxycarbonyl)-5-(ethoxycarbonyl)pyrrolidine-2-carboxylic acid

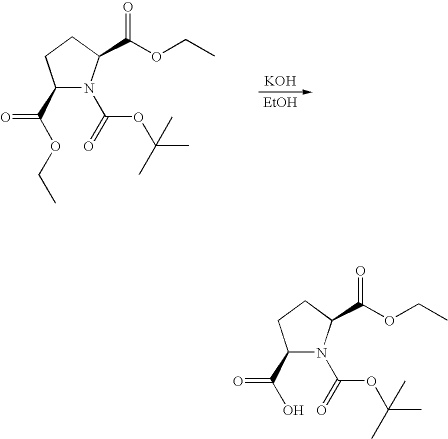

To a solution of (±)-1-tert-butyl 2,5-diethyl (2R,5S)-pyrrolidine-1,2,5-tricarboxylate (2.1 g, 6.66 mmol) in EtOH (4 mL), a solution of potassium hydroxide (0.373 g, 6.66 mmol) in EtOH (4 mL) was added dropwise. After being stirred at room temperature for 2 h, the reaction mixture was concentrated in vacuo. The residue was diluted with water and washed with ether. The aqueous layer was acidified with 3N HCl to pH2˜3 and extracted with ether. The ether layer was dried and concentrated to give the title compound (1.1 g, 57.6%, pale yellow sticky oil). ¹H NMR (CDCl₃), δ (ppm): 4.25-4.70 (m, 4H), 2.0-2.6 (m, 4H), 1.47 (s, 9H), 1.35 (t, 3H).

### (viii) (±)-1-tert-butyl 2-ethyl (2S,5R)-5-(aminocarbonyl)pyrrolidine-1,2-dicarboxylate

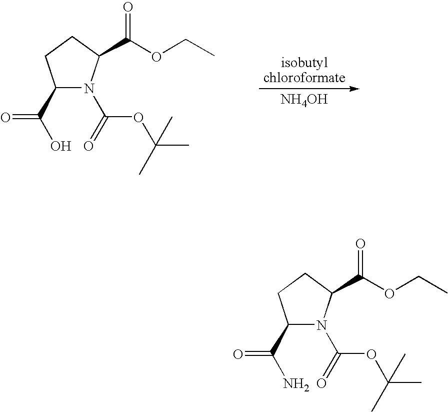

Isobutyl chloroformate (548 mg, 4.02 mmol) was added dropwise to a cold (−50° C.) solution of (±)-(2R,5S)-1-(tert-butoxycarbonyl)-5-(ethoxycarbonyl)pyrrolidine-2-carboxylic acid (1.1 g, 3.83 mmol) in THF (20 mL) and Et₃N (2.5 mL). The reaction mixture was warmed to 0° C. and stirred for 1 h, and concentrated ammonium hydroxide (20 mL) was added. After being stirred for another 0.5 h, the reaction mixture was diluted with water and extracted with ethyl acetate. The organic layer was dried with MgSO₄ and concentrated in vacuo to give the amide intermediate (1.1 g, 100%, pale yellow sticky oil). ¹H NMR (CDCl₃), δ (ppm): 8.08 & 7.82 (ws, 1H), 5.40 & 5.43 (ws, 1H), 4.42 (m, 1H), 4.25 (m, 3H), 1.90-2.40 (m, 4H), 1.45 (d, 9H), 1.33 (t, 3H).

### (ix) (±)-1-tert-butyl 2-ethyl (2S,5R)-5-cyanopyrrolidine-1,2-dicarboxylate

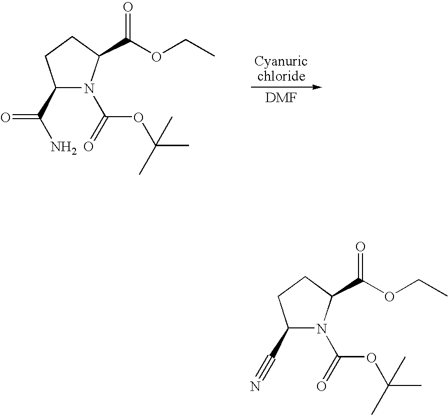

(±)-1-tert-butyl 2-ethyl (2S,5R)-5-(aminocarbonyl)pyrrolidine-1,2-dicarboxylate (1.1 g, 3.83 mmol) was stirred with cyanuric chloride (0.425 g, 2.3 mmol) in DMF (3.5 mL) at room temperature for 1 h. The reaction mixture was quenched with water and extracted with ethyl acetate. The organic layer was washed with water and brine, dried with MgSO4 and concentrated to give the title compound (0.9 g, 87.3%, colorless oil). ¹H NMR (CDCl₃), δ (ppm): 4.66 & 4.56(m, 1H), 4.40 & 4.22 (m, 3H), 2.12-2.50 (m, 4H), 1.53 & 1.45 (s, 9H), 1.30 (t, 3H).

### (x) (±)-1-tert-butyl 2-ethyl (2S,5R)-5-(2H-tetrazol-5-yl)pyrrolidine-1,2-dicarboxylate

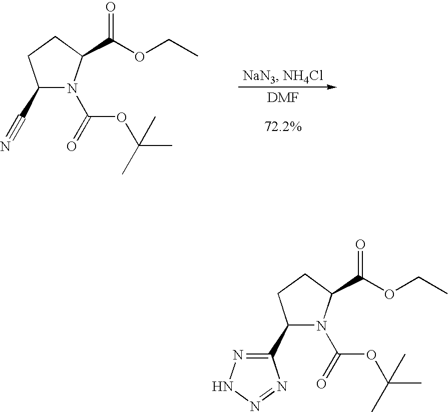

(±)-1-tert-butyl 2-ethyl (2S,5R)-5-cyanopyrrolidine-1,2-dicarboxylate (896 mg, 3.34 mmol) was heated with sodium azide (239 mg, 3.67 mmol) and ammonium chloride (196 mg, 3.67 mmol) in DMF at 110° C. overnight in a sealed tube. The reaction mixture was quenched with water and extracted with ethyl acetate. The organic layer was washed with water and brine, dried with MgSO4, concentrated to give the title compound (750 mg, 72.2%, pale-yellow sticky oil). ¹H NMR (CDCl₃), δ (ppm): 5.54 & 5.46 (m, 1H), 4.96 & 4.18-4.50 (m, 3H), 1.90-2.60 (m, 4H), 1.20-1.50 (m, 12H). When this reaction was repeated at 115° C. for 36 h, the yield improved to 84%.

### (xi) (±)-Tert-butyl (2R,5S)-2-[2-(3-chlorophenyl)-2H-tetrazol-5-yl]-5-(ethoxycarbonyl)pyrrolidine-1-carboxylate

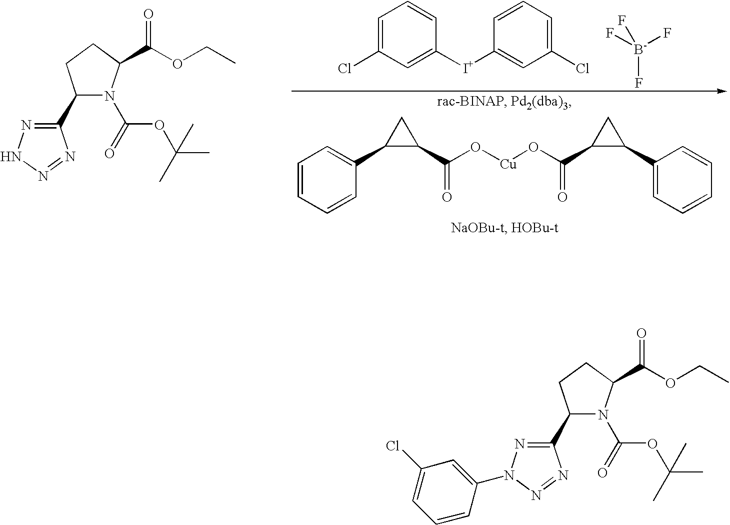

1-Tert-butyl 2-ethyl (2S,5R)-5-(2H-tetrazol-5-yl)pyrrolidine-1,2-dicarboxylate (600 mg, 1.92 mmol), bis-(3-chloro-phenyl)-iodonium tetrafluoro borate (1.17 g, 2.68 mmol), sodium tert-butoxide (185 mg, 1.92 mmol), (±)-BINAP (48 mg, 0.077 mmol), Pd₂(dba)₃ (18 mg, 0.019 mmol) and copper 2-phenylcyclopropanecarboxylate (15 mg, 0.039 mmol) were mixed in tert-butanol (40 mL) under argon and heated at 90° C. for 36 h. The reaction mixture was then concentrated in vacuo. Chromatography (silica, 8-20% ethyl acetate in hexanes) gave the title compound (612 mg, 75%, pale-yellow oil). ¹H NMR (CDCl₃), δ (ppm): 8.18 (s, 1H), 8.07 (d, 1H), 7.49 (m, 2H), 5.44 & 5.31 (m, 1H), 4.52, & 4.42 (m, 1H), 4.20 (m, 2H), 2.42 (m, 4H), 1.44 & 1.32 (s, 9H), 1.27 (t, 3H).

In a similar manner, using degassed solvent with only 2 h heating, the following compound was synthesized:

| example | Structure | Name | Yield |
|---|---|---|---|
| 2.2 xi | 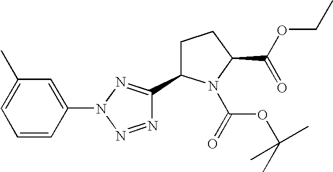 | (±)-1-Tert-butyl (2S,5R)-2-(ethoxycarbonyl)-5-[2-(3-methylphenyl)-2H-tetrazol-5-yl]pyrrolidine-1-carboxylate | 74%,yellowoil |

| | |
|---|---|
| NMR | 7.95-7.91 (m, 2 H), 7.41 (m, 1 H), 7.27 (m, 1 H), 5.43 (bs, 1 H), 5.29 (bs, 1 H), 4.50 (bs, 1 H), |
| | 4.39 (bs, 1 H), 4.19 (m, 2 H), 2.46 (s, 3 H), 2.38 (m, 2 H), 1.59 (s, 3 H), 1.42 (s, 3 H), 1.34 (s, |
| | 3 H), 1.26 (t, 3 H). |

### (xii) (±)-ethyl (2S,5R)-5-[2-(3-chlorophenyl)-2H-tetrazol-5-yl]pyrrolidine-2-carboxylate

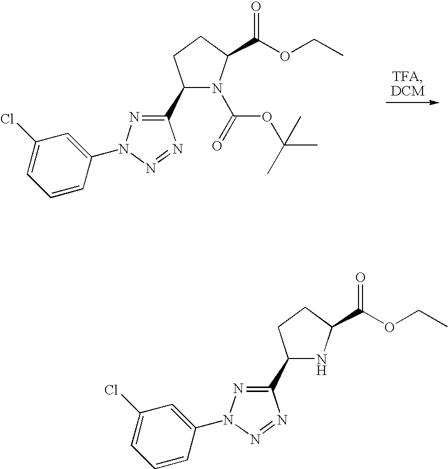

(±)-Tert-butyl (2R,5S)-2-[2-(3-chlorophenyl)-2H-tetrazol-5-yl]-5-(ethoxycarbonyl)pyrrolidine-1-carboxylate (365 mg, 0.865 mmol) was mixed with TFA (1.2 mL) and DCM (1.2 mL) at 0° C. and stirred at room temperature for 15 min. The reaction mixture was diluted with DCM and quenched with 2M Na₂CO₃. The organic layer was dried with MgSO₄, concentrated to give the title compound (227 mg, 81.7%, pale-yellow sticky oil). ¹H NMR (CDCl₃), δ (ppm): 8.18 (s, 1H), 8.06 (d, 1H), 7.49 (m, 2H), 4.68 (m, 1H), 4.18 (q, 2H), 3.99 (m, 1H), 2.10-2.50 (m, 4H), 1.29 (t, 3H). When the reaction was repeated, 98% yield was obtained.

In a similar manner the following compound was synthesized:

| example | Structure | Name | Yield |
|---|---|---|---|
| 2.2 xii | 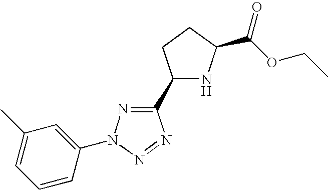 | (±)-ethyl (2S,5R)-5-[2-(3-methylphenyl)-2H-tetrazol-5-yl]pyrrolidine-2-carboxylate | 96%,yellowoil |

| | |
|---|---|
| NMR | 7.93-7.89 (m, 2 H), 7.43 (m, 1 H), 7.29 (m, 1 H), 4.69 (t, 1 H), 4.22 (q, 2 H), 4.13 (m, 1 H), |
| | 4.01 (m, 1 H), 2.46 (s, 3 H), 2.39 (m, 2 H), 2.22 (m, 2 H), 1.28 (t, 3 H). |

### (xiii) (±)-(6R,8aS)-6-[2-(3-chlorophenyl)-2H-tetrazol-5-yl]hexahydropyrrolo[1,2-a]pyrazine-1,4-dione

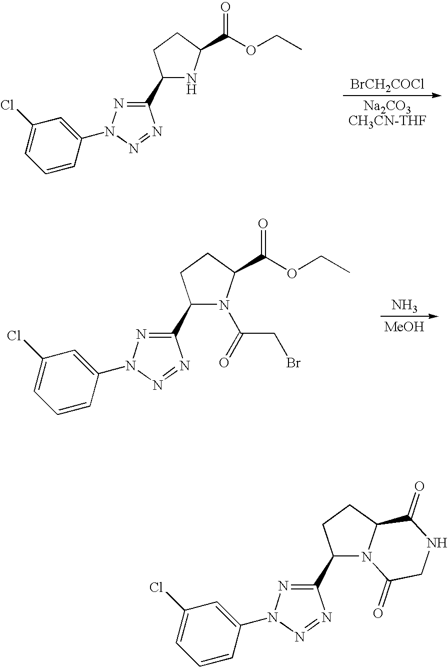

(±)-Ethyl (2S,5R)-5-[2-(3-chlorophenyl)-2H-tetrazol-5-yl]pyrrolidine-2-carboxylate (460 mg, 1.43 mmol) was mixed with bromoacetyl chloride (0.15 mL, 1.80 mmol) and Na₂CO₃ (607 mg, 5.73 mmol) in CH₃CN (3 mL) and THF (1 mL) at −50° C. Then the reaction mixture was warmed to 0° C. and stirred for 30 min. The reaction was cooled to −78° C. and quenched with NH₃/MeOH (7N) (1.18 mL, 8.26 mmol), warmed to room temperature and stirred for 1 h. The reaction mixture was concentrated under vacuum, the residue was diluted in water, extracted with ethyl acetate (3×50 mL). The organic layer was dried over MgSO₄, the solvent was removed in vacuo and the residue was purified by column chromatography with 2% MeOH in DCM to give the title compound (380 mg, 80%, white solid). ¹H NMR (CDCl₃), δ (ppm): 8.12 (s, 1H), 8.01 (d, 1H), 7.50 (m, 2H), 6.19 (m, 1H), 5.60 (m, 1H), 4.40 (m, 1H), 4.18 d, 1H), 4.93 (dd, 1H), 2.36 (m, 3H), 2.24 (m, 1H).

In a similar manner the following compound was synthesized:

| example | Structure | Name | Yield |
|---|---|---|---|
| 2.2 xiii | 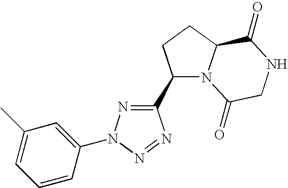 | (±)-(6R,8aS)-6-[2-(3-methylphenyl)-2H-tetrazol-5-yl]hexahydropyrrolo[1,2-a]pyrazine-1,4-dione | 96%,whitesolid |

| | |
|---|---|
| NMR | 7.79-7.76 (m, 2 H), 7.33-7.18 (m, 2 H), 5.50 (m, 1 H), 4.28 (bs, 1H), 4.06 (m, 1H), 3.86 (m, |
| | 1 H), 2.36 (s, 3 H), 2.15 (m, 1 H), 1.34 (m, 4 H). |

### (xiv) (±)-(6R,8aS)-6-[2-(3-chlorophenyl)-2H-tetrazol-5-yl]octahydropyrrolo[1,2-a]pyrazine

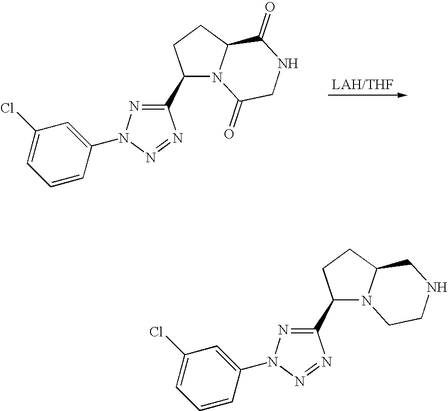

(±)-(6R,8aS)-6-[2-(3-chlorophenyl)-2H-tetrazol-5-yl]hexahydropyrrolo[1,2-a]pyrazine-1,4-dione (102 mg, 0.307 mmol) was mixed with LiAlH₄ (0.921 mL, 1M, 0.921 mmol) in THF at 50° C. for 15 min. The reaction mixture was quenched with saturated sodium sulfate solution at 0° C. until no gas released, diluted with ethyl acetate and filtered. The filtrate was concentrated and purified by column chromatography with 2.5-5% MeOH (2M NH₃) in DCM to give the title compound (40 mg, 42.7%, yellow sticky oil). ¹H NMR (CDCl₃), δ (ppm): 8.17 (s, 1H), 8.04 (d, 1H), 7.47 (m, 2H), 3.76 (t, 1H), 3.18 (d, 1H), 3.06 (d, 1H), 2.84 (dt, 1H), 2.70 (t, 1H), 2.30 (m, 5H), 1.96 (m, 1H), 1.68 (m, 1H).

In a similar manner the following compound was synthesized:

| example | Structure | Name | Yield |
|---|---|---|---|
| 2.2 | 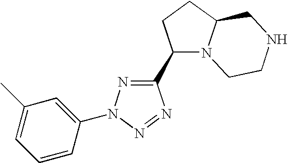 | (±)-(6R,8aS)-6-[2-(3-methylphenyl)-2H-tetrazol-5-yl]octahydropyrrolo[1,2-a]pyrazine | 25%,yellowoil |

| | |
|---|---|
| NMR | 7.94 (m, 2 H), 7.42-7.27 (m, 2 H), 3.94-2.61 (m, 4 H), 3.41-1.25 (m, 9 H), 2.47 (s, 3 H). |

### Example 3.1

### (±)-Tert-butyl (6R,8aS)-6-[5-(3-chlorophenyl)isoxazol-3-yl]hexahydropyrrolo[1,2-a]pyrazine-2(1H)-carboxylate

### i) (±)-Tert-butyl (6R,8aS)-6-formylhexahydropyrrolo[1,2-a]pyrazine-2(1H)-carboxylate

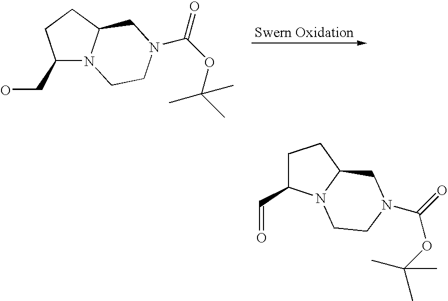

To a solution of oxalyl chloride (2M, 2.8 mL, 5.6 mmol) in DCM (9 mL) was added DMSO (0.58 mL, 8 mmol) at −78° C. After stirring 10 min, a solution of (±)-tert-butyl (6R,8aS)-6-(hydroxymethyl)hexahydropyrrolo[1,2-a]pyrazine-2(1H)-carboxylate (696 mg, 2.7 mmol) in DCM (5 mL) was added. After stirring at −78° C. for 1 h, Et₃N (2 mL) was added. The mixture was stirred at room temperature for 30 min, then poured into DCM (40 mL)/NH₃—H₂O (10%, 15 mL). The organic phase was separated, dried over Na₂SO₄ and concentrated to give crude aldehyde.

### ii) (±)-Tert-butyl (6R,8aS)-6-[3-(3-chlorophenyl)-1-hydroxyprop-2-yn-1-yl]hexahydropyrrolo[1,2-a]pyrazine-2(1H)-carboxylate

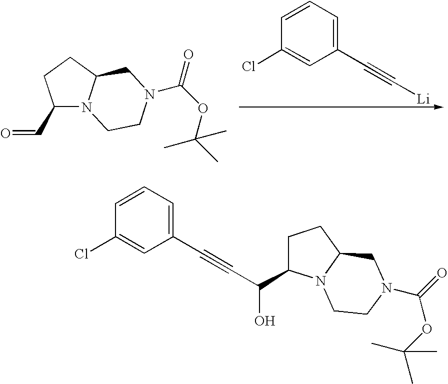

The aldehyde was diluted with THF (10 mL) and cooled to −40° C. 3-Chlorophenylacetylenelithium [generated from corresponding acetylene (0.533 mL, 4.3 mmol), butyllithium (2.5N in pentane, 1.72 mL, 4.3 mmol) and THF (6 mL)] was added over 5 min. Saturated NH₄Cl (10 mL) was added to the resulting mixture and the product was extracted with ethyl acetate. The combined extracts were dried, concentrated and purified on silica gel column to give the corresponding alcohol (814 mg, 77%).

### iii) (±)-Tert-butyl (6R,8aS)-6-[3-(3-chlorophenyl)prop-2-ynoyl]hexahydropyrrolo[1,2-a]pyrazine-2(1H)-carboxylate

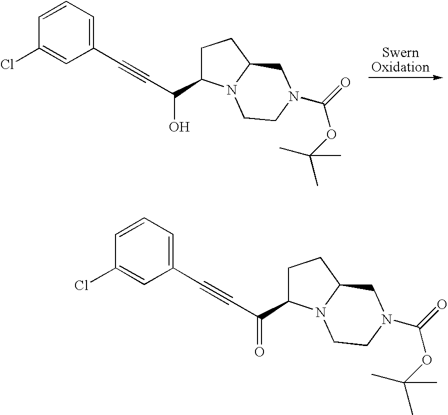

The alcohol (392 mg, 1 mmol) was oxidized with Swern oxidation following the same procedure as example 3 i) and purified on silica gel column to give pure ketone product (275 mg, 70%).

### iv) (±)-Tert-butyl (6R,8aS)-6-[5-(3-chlorophenyl)isoxazol-3-yl]hexahydropyrrolo[1,2-a]pyrazine-2(1H)-carboxylate

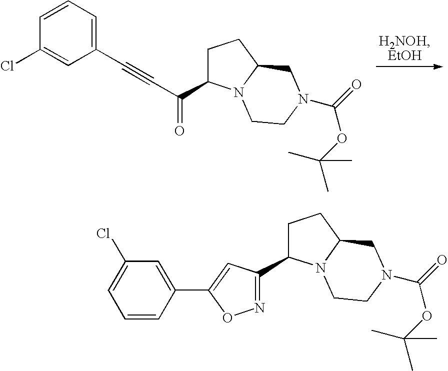

A mixture of ketone (95 mg, 0.24 mmol), H₂NOH.HCl (22 mg, 0.3 mmol), Na₂CO₃ (17 mg, 0.16 mmol) and EtOH (1.5 mL) was stirred at room temperature for 4 days. After removal of EtOH, ethyl acetate was added and the organic solution was washed with Na₂CO₃ aq. After concentration, the residue was purified on silica gel column to give intermediate isoxazole (89 mg, 92%). ¹H NMR (CDCl₃), δ (ppm): 1.48 (s, 9H), 1.58 (m, 1H), 1.85-2.38 (m, 5H), 2.65-2.85 (m, 3H), 3.57 (dd, 1H), 4.05-4.25 (br, 2H), 6.60 (s, 1H), 7.42 (m, 2H), 7.67 (m, 1H), 7.78 (s, 1H).

In a similar manner the following compounds were synthesized:

| | | | Yield |
|---|---|---|---|
| example | Structure | Name | 4 steps |
| 3.2 | 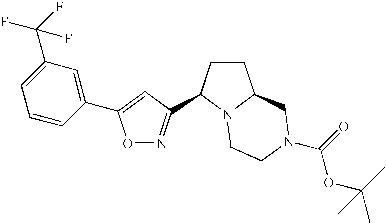 | (±)tert-butyl(6R,8aS)-6-{5-[3-(trifluoromethyl)phenyl]isoxazol-3-yl}hexahydropyrrolo[1,2-a]pyrazine-2(1H)-carboxylate | 35% |
| NMR | 1.46 (s, 9 H), 1.58 (m, 1 H), 1.80-1.98 (m, 2 H), 2.08-2.31 (m, 3 H), 2.48-2.91 | | |
| | (m, 3 H), 3.57 (dd, 1 H), 3.90-4.36 (m, 2 H), 6.65 (s, 1 H), 7.61 (dd, 1 H), 7.69 | | |
| | (d, 1H), 7.98 (d, 1 H), 8.03 (s, 1 H). | | |
| 3.3 | 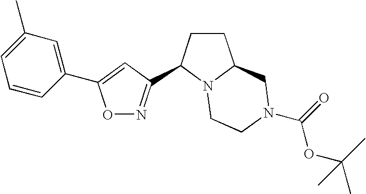 | (±)tert-butyl(6R,8aS)-6-[5-(3-methylphenyl)isoxazol-3-yl]hexahydropyrrolo[1,2-a]pyrazine-2(1H)-carboxylate | 35% |
| NMR | 1.47 (s, 9 H), 1.52-1.64 (m, 1 H), 1.80-1.96 (m, 2 H), 2.06-2.16 (m, 1 H), 2.16-2.27 (m, 2H), | | |
| | 2.41 (s, 3 H), 2.54-2.91 (m, 3 H), 3.54 (dd, 1 H), 3.88-4.37 (m, 2 H), 6.53 (s, 1 H), 7.22-7.26 | | |
| | (d, 1 H), 7.31-7.37 (dd, 1 H), 7.56-7.62 (m, 2 H). | | |
| 3.4 | 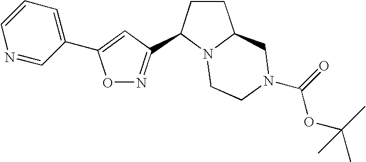 | (±)tert-butyl(6R,8aS)-6-(5-pyridin-3-ylisoxazol-3-yl)hexahydropyrrolol[1,2-a]pyrazine-2(1H)-carboxylate | 30% |
| NMR | 1.46 (s, 9 H), 1.59 (m, 1 H), 1.80-1.99 (m, 2 H), 2.07-2.30 (m, 3 H), 2.52-2.90 (m, 3 H), 3.58 | | |
| | (dd, 1 H), 3.88-4.36 (m, 2 H), 6.69 (s, 1 H), 7.42 (dd, 1 H), 8.09 (d, 1 H), 8.67 (d, 1 H), 9.03 (s, | | |
| | 1 H). | | |
| 3.5 | 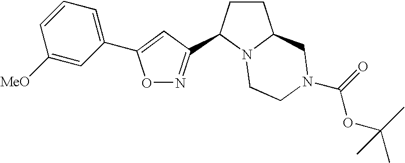 | (±)tert-butyl(6R,8aS)-6-[5-(3-methoxyphenyl)isoxazol-3-yl]hexahydropyrrolo[1,2-a]pyrazine-2(1H)-carboxylate | 16% |
| NMR | 1.41 (s, 9 H), 1.51 (m, 1 H), 1.78-1.89 (m, 2 H), 1.98-2.20 (m, 3 H), 2.42-2.88 (m, 3 H), 3.46 | | |
| | (dd, 1 H), 3.77 (s, 1 H), 3.83-4.30 (m, 2 H), 6.49 (s, 1 H), 6.86-6.90 (m, 1 H), 7.22 (s, 1 H), 7.28 | | |
| | (m, 2 H). | | |
| 3.6 | 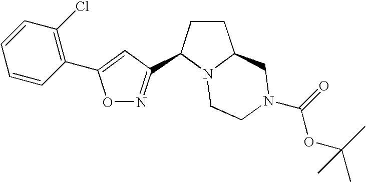 | (±)tert-butyl(6R,8aS)-6-[5-(2-chlorophenyl)isoxazol-3-yl]hexahydropyrrolo[1,2-a]pyrazine-2(1H)-carboxylate | 21% |
| NMR | 1.46 (s, 9 H), 1.54-1.66 (m, 1 H), 1.80-2.00 (m, 2 H), 2.04-2.30 (m, 3 H), 2.50-2.98 (m, 3 H), | | |
| | 3.58 (dd, 1 H), 3.88-4.34 (m, 2 H), 6.97 (s, 1 H), 7.28-7.42 (m, 2 H), 7.52 (m, 1 H), 7.95 (m, | | |
| | 1 H). | | |
| 3.7 | 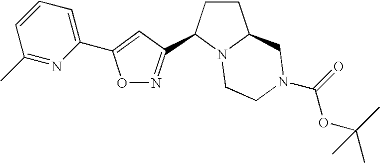 | (±)tert-butyl(6R,8aS)-6-[5-(6-methylpyridin-2-yl)isoxazol-3-yl]hexahydropyrrolo[1,2-a]pyrazine-2(1H)-carboxylate | 18% |
| NMR | 1.46 (s, 9 H), 1.52-1.64 (m, 1 H), 1.80-1.94 (m, 2 H), 2.06-2.26 (m, 3 H), 2.48-2.64 (m, 1 H), | | |
| | 2.62 (s, 3 H), 2.74-2.90 (m, 2 H), 3.58 (dd, 1 H), 3.88-4.34 (m, 2 H), 6.94 (s, 1 H), 7.19 (dd, | | |
| | 1 H), 7.70 (m, 2 H). | | |
| 3.8 | 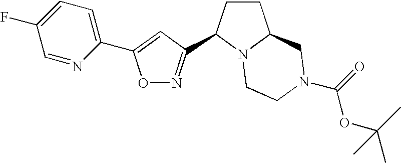 | (±)tert-butyl(6R,8aS)-6-[5-(5-fluoropyridin-2-yl)isoxazol-3-yl]hexahydropyrrolo[1,2-a]pyrazine-2(1H)-carboxylate | 5% |

| | |
|---|---|
| NMR | 1.46 (s, 9 H), 1.54-1.72 (m, 1 H), 1.80-1.96 (m, 2 H), 2.06-2.30 (m, 3 H), 2.51-2.88 (m, 3 H), |
| | 3.59 (dd, 1 H), 3.88-4.35 (m, 2 H), 6.89 (s, 1 H), 7.54 (m, 1 H), 7.92 (dd, 1 H), 8.54 (d, 1 H). |

### Example 4.1

### (±)-2-[(6R,8aS)-6-[1-(3-fluorophenyl)-1H-1,2,3-triazol-4-yl]hexahydropyrrolo[1,2-a]pyrazin-2(1H)-yl]nicotinonitrile

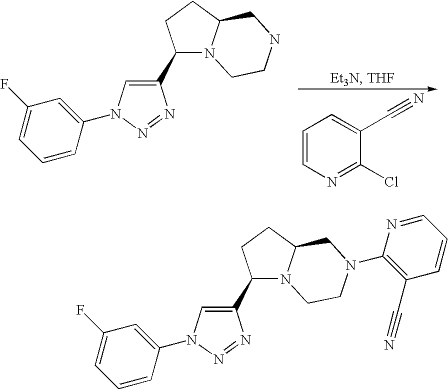

A mixture of (±)-(6R,8aS)-6-[1-(3-fluorophenyl)-1H-1,2,3-triazol-4-yl]octahydropyrrolo[1,2-a]pyrazine (58 mg, 0.2 mmol), 2-chloronicotinonitrile (55 mg, 0.4 mmol), Et₃N (0.1 mL) and THF (1.5 mL) was stirred at 80° C. for 4 h. The resulting mixture was concentrated and purified on silica gel column to afford the product (58 mg, 75%). ¹H NMR (300 MHz, CDCl₃): δ (ppm) 1.73 (m, 1H), 1.99 (m, 2H), 2.4 (m, 3H), 2.95-3.18 (m, 3H), 3.71 (dd, 1H), 4.38-4.59 (m, 2H), 6.76 (dd, 1H), 7.16 (m, 1H), 7.55 (m, 3H), 7.76 (dd, 1H), 7.96 (s, 1H), 8.37 (d, 1H).

In a similar manner the following compounds were synthesized:

| Example | Structure | Name | Yield |
|---|---|---|---|
| 4.2 | 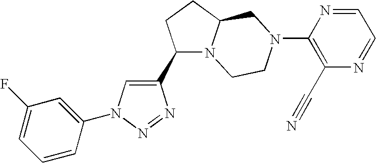 | (±)-3-[(6R,8aS)-6-[1-(3-fluorophenyl)-1H-1,2,3-triazol-4-yl]hexahydropyrrolo[1,2-a]pyrazin-2(1H)-yl]pyrazine-2-carbonitrile | 86% |
| NMR | 1.72 (m, 1 H), 2.01 (m, 2 H), 2.38 (m, 3 H), 3.01- 3.24 (m, 3 H), 3.72 (dd, 1 H), 4.15-4.7 (m, | | |
| | 2 H), 7.16 (m, 1 H), 7.55 (m, 3 H), 7.96 (s, 1 H), 8.02 (d, 1 H), 8.27 (s, 1 H). | | |
| 4.3 | 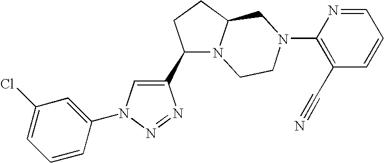 | (±)-2-[(6R,8aS)-6-[1-(3-chlorophenyl)-1H-1,2,3-triazol-4-yl] hexahydropyrrolo[1,2-a]pyrazin-2(1H)-yl]nicotinonitrile | 73% |
| NMR | 1.69 (m, 1 H), 1.99 (m, 2 H), 2.44 (m, 3 H), 2.95-3.18 (m, 3 H), 3.72 (dd, 1 H), 4.41-4.59 | | |
| | (m, 2 H), 6.76 (dd, 1 H), 7.46 (m, 2 H), 7.68-7.82 (m, 3 H), 7.96 (s, 1 H), 8.37 (d, 1 H). | | |
| 4.4 | 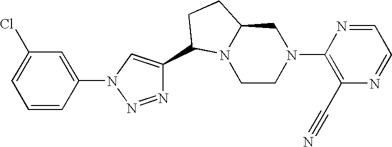 | (±)-3-[(6R,8aS)-6-[1-(3-chlorophenyl)-1H-1,2,3-triazol-4-yl]hexahydropyrrolo[1,2-a]pyrazin-2(1H)-yl]pyrazine-2-carbonitrile | 75% |
| NMR | 1.75 (m, 1 H), 2.01 (m, 2 H), 2.38 (m, 3 H), 3.01-3.23 (m, 3 H), 3.72 (dd, 1 H), 4.50-4.7 (m, | | |
| | 2 H), 7.46 (m, 2 H), 7.68-7.82 (m, 2 H), 7.96 (s, 1 H), 8.03 (d, 1 H), 8.37 (d, 1 H). | | |
| 4.5 | 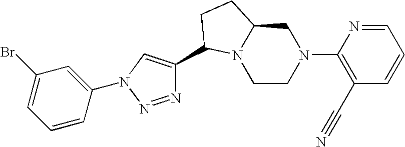 | (±)-2-[(6R,8aS)-6-[1-(3-bromophenyl)-1H-1,2,3-triazol-4-yl]hexahydropyrrolo[1,2-a]pyrazin-2(1H)-yl]nicotinonitrile | 60% |
| NMR | 1.71 (m, 1 H), 1.99 (m, 2 H), 2.40 (m, 3 H), 2.95-3.18 (m, 3 H), 3.72 (dd, 1 H), 4.41-4.59 | | |
| | (m, 2 H), 6.76 (dd, 1 H), 7.42 (dd, 1 H), 7.57 (d, 1 H), 7.77 (m, 2 H), 7.96 (m, 2 H), 8.36 (d, | | |
| | 1 H). | | |
| 4.6 | 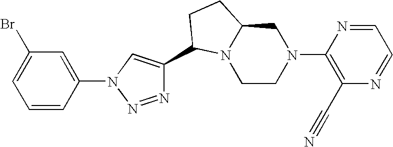 | (±)-3-[(6R,8aS)-6-[1-(3-bromophenyl)-1H-1,2,3-triazol-4-yl]hexahydropyrrolo[1,2a]pyrazin-2(1H)-yl]pyrazine-2-carbonitrile | 72% |
| NMR | 1.73 (m, 1 H), 2.01 (m, 2 H), 2.38 (m, 3 H), 3.01- 3.23 (m, 3 H), 3.72 (dd, 1 H), 4.49-4.7 (m, | | |
| | 2 H), 7.42 (dd, 1 H), 7.57 (d, 1 H), 7.73 (d, 1 H), 7.96 (s, 1 H), 8.03 (d, 1 H), 8.27 (d, 1 H). | | |
| 4.7 | 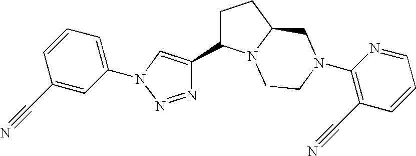 | (±)-2-[(6R,8aS)-6-[1-(3-cyanophenyl)-1H-1,2,3-triazol-4-yl]hexahydropyrrolo[1,2-a]pyrazin-2(1H)-yl]nicotinonitrile | 39% |
| NMR | 1.71 (m, 1 H), 2.05 (m, 2 H), 2.42 (m, 3 H), 2.95-3.17 (m, 3 H), 3.73 (dd, 1 H), 4.38-4.59 | | |
| | (m, 2 H), 6.76 (dd, 1 H), 7.73 (m, 3 H), 8.01 (s, 1 H), 8.10 (m, 2 H), 8.36 (d, 1 H). | | |
| 4.8 | 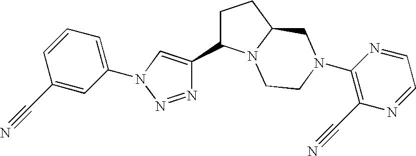 | (±)-3-[(6R,8aS)-6-[1-(3-cyanophenyl)-1H-1,2,3-triazol-4-yl]hexahydropyrrolo[1,2-a]pyrazin-2(1H)-yl]pyrazine-2-carbonitrile | 48% |

| | |
|---|---|
| NMR | 1.73 (m, 1 H), 2.04 (m, 2 H), 2.4 (m, 3 H), 3.00-3.23 (m, 3 H), 3.74 (dd, 1 H), 4.49-4.7 (m, |
| | 2 H), 7.74 (m, 2 H), 8.08- (d, 8.11 (m, 4 H), 8.27 (d, 1 H). |

### Example 5.1

### (±)-3-[(6R,8aS)-6-[2-(3-chlorophenyl)-2H-tetrazol-5-yl]hexahydropyrrolo[1,2-a]pyrazin-2(1H)-yl]pyrazine-2-carbonitrile

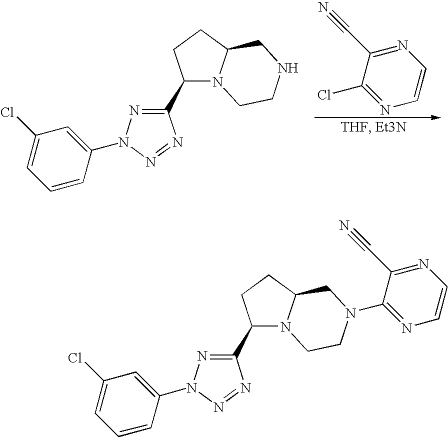

(±)-(6R,8aS)-6-[2-(3-chlorophenyl)-2H-tetrazol-5-yl]octahydropyrrolo[1,2-a]pyrazine (40 mg, 0.131 mmol) was mixed with 3-chloropyrazine-2-carbonitrile (23.8 mg, 0.17 mmol) and Et₃N (0.1 mL) in THF (1 mL) in a sealed vial and heated at 90° C. for 30 min. The reaction mixture was quenched with water and extracted with DCM. The product was purified by column chromatography with 20% ethyl acetate in hexanes to give the title compound (23.8 mg, 44.5%, yellow foam). ¹H NMR (CDCl₃), δ (ppm): 8.27 (s, 1H), 8.26 (s, 1H), 8.08 (d, 1H), 8.03 (s, 1H), 7.49 (m, 2H), 4.69 (d, 1H), 4.52 (d, 1H), 3.86 (t, 1H), 3.30 (d, 1H), 3.22 (d, 1H), 3.11 (dd, 1H), 2.32-2.60 (m, 4H), 2.04 (m, 1H), 1.84 (m, 1H).

In a similar manner, but heating at reflux overnight, the following compounds were synthesized:

| example | Structure | Name | Yield |
|---|---|---|---|
| 5.2 | 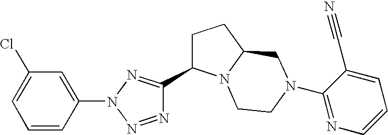 | (±)-2-[(6R,8aS)-6-[2-(3-chlorophenyl)-2H-tetrazol-5-yl]hexahydropyrrolo[1,2-a]pyrazin-2(1H)-yl]nicotinonitrile | 20%,whitesolid |
| NMR | 8.34 (m, 1H), 8.19 (m, 1H), 8.08 (m, 1H), 7.78 (m, 1H), 7.52 (m, 2H), 6.76 (q, 1H), 4.59 (dt, 1H), 4.42 (m, 1H), | | |
| | 3.86 (t, 1H), 3.25 (m, 2H), 3.08 (m, 1H), 2.59-2.35 (m, 4H), 2.07 (m, 1H), 1.84 (m, 1H). | | |
| 5.3 | 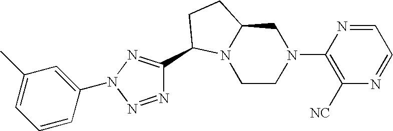 | (±)-3-[(6R,8aS)-6-[2-(3-methylphenyl)-2H-tetrazol-5-yl]hexahydropyrrolo[1,2-a]pyrazin-2(1H)-yl]pyrazine-2-carbonitrile | 24%,oil |
| NMR | 8.26 (m, 1H), 8.01 (m, 1H), 7.96 (m, 2H), 7.45 (m, 1H), 7.31 (t, 1H), 4.70 (dd, 1H), 4.53 (d, 1H), 3.86 (q, 1H), | | |
| | 3.30 (m, 2H), 3.13 (m, 1H), 2.47 (s, 3H), 2.52-2.34 (m, 4H), 2.07 (m, 1H), 1.83 (m, 1H). | | |
| 5.4 | 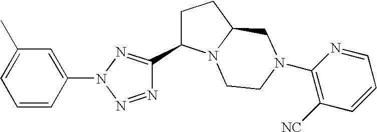 | (±)-2-[(6R,8aS)-6-[2-(3-methylphenyl)-2H-tetrazol-5-yl]hexahydropyrrolo[1,2-a]pyrazin-2(1H)-yl]nicotinonitrile | 39% |

| | |
|---|---|
| NMR | 8.34 (m, 1H), 7.96 (t, 2H), 7.77 (m, 1H), 7.44 (t, 1H), 7.29 (d, 1H), 6.75 (m, 1H), 4.60 (m, 1H), 4.42 (m, 1H), |
| | 3.85 (t, 1H), 3.25 (m, 2H), 3.08 (m, 1H), 2.59-2.31 (m, 4H), 2.47 (s, 3H), 2.07 (m, 1H), 1.84 (m, 1H). |

example Structure Name Yield 5.2 (±)-2-[(6R,8aS)-6-[2-(3-20%, C1<CWIJC umer= 236><Nt g NCN?=chlorophenyl)-2H-tetrazol-5-white N N Nyl]hexahydropyrrolo[1,2-a]pyrazin-solid 2(1H)-yl]nicotinonitrile NMR 8.34 (m, 1H), 8.19 (m, 1H), 8.08 (m, 1H), 7.78 (m, 1H), 7.52 (m, 2H), 6.76 (q, 1H), 4.59 (dt, 1H), 4.42 (m, 1H), 3.86 (t, 1H), 3.25 (m, 2H), 3.08 (m, 1H), 2.59-2.35 (m, 4H), 2.07 (m, 1H), 1.84 (m, 1H). 5.3 (CW™-a 237(±)-3-[(6R,8aS)-6-[2-(3-24%, <N^{m}j)N/¹>methylphenyl)-2H-tetrazol-5-oil XN N>N NC yl]hexahydropyrrolo[1,2-a]pyrazin-2(1H)-yl]pyrazine-2-carbonitrile NMR 8.26 (m, 1H), 8.01 (m, 1H), 7.96 (m, 2H), 7.45 (m, 1H), 7.31 (t, 1H), 4.70 (dd, 1H), 4.53 (d, 1H), 3.86 (q, 1H), 3.30 (m, 2H), 3.13 (m, 1H), 2.47 (s, 3H), 2.52-2.34 (m, 4H), 2.07 (m, 1H), 1.83 (m, 1H). 5.4<C 82/(−)-2-[(6R,8aS)-6-[2-(3-39%<Nt Ntyg) methylphenyl)-2H-tetrazol-5-a=/N>N NC yl]hexahydropyffolo[1,2-a]pyrazin-12(1H)-yl]nicotinonitrile 8.34 (m, 1H), 7.96 (t, 2H), 7.77 (m, 1H), 7.44 (t, 1H), 7.29 (d, 1H), 6.75 (m, 1H), 4.60 (m, 1H), 4.42 (m, 1H), 3.85 (t, 1H), 3.25 (m, 2H), 3.08 (m, 1H), 2.59-2.31 (m, 4H), 2.47 (s, 3H), 2.07 (m, 1H), 1.84 (m, 1H).

### Example 6.1

### (±)-3-[(6R,8aS)-6-[5-(3-chlorophenyl)isoxazol-3-yl]hexahydropyrrolo[1,2-a]pyrazin-2(1H)-yl]pyrazine-2-carbonitrile

### i) (±)-(6R,8aS)-6-[5-(3-chlorophenyl)isoxazol-3-yl]octahydropyrrolo[1,2-a]pyrazine

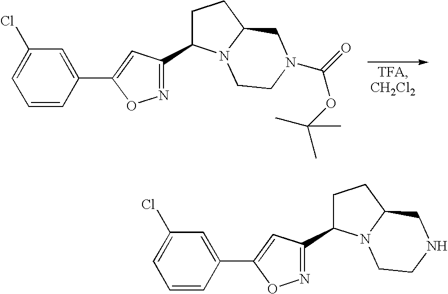

This Boc protected intermediate was treated with TFA (0.5 mL) and DCM (1 mL) at room temperature for 2 h. Standard work-up as above provided (O)-(6R,8aS)-6-[5-(3-chlorophenyl)isoxazol-3-yl]octahydropyrrolo[1,2-a]pyrazine (66 mg). The crude product was used without further purification.

### ii)

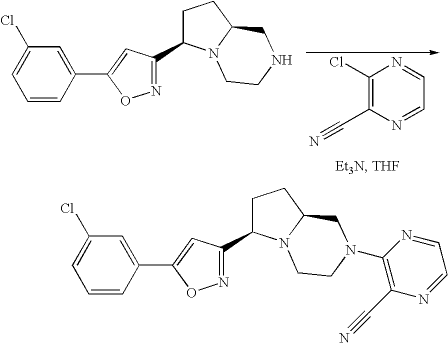

(±)-(6R,8aS)-6-[5-(3-chlorophenyl)isoxazol-3-yl]octahydropyrrolo[1,2-a]pyrazine (66 mg) (33 mg, 0.11 mmol), 3-chloropyrazine-2-carbonitrile (28 mg, 0.2 mmol), Et₃N (1.5 mL) and THF (0.1 mL) was stirred at 80° C. overnight. After concentration, the crude product was directly subjected to silica gel column to afford pure product (38 mg, 84% over two steps). ¹H NMR (300 MHz, CDCl₃): δ (ppm) 1.75 (m, 1H), 1.83-2.45 (m, 5H), 2.96-3.24 (m, 3H), 3.65 (dd, 1H), 4.14-4.7 (m, 2H), 6.63 (s, 1H), 7.42 (m, 2H), 7.69 (m, 1H), 7.79 (s, 1H), 8.02 (d, 1H), 8.27 (d, 1H).

In a similar manner the following compounds were synthesized:

| Example | Structure | Name | Yield |
|---|---|---|---|
| 6.2 | 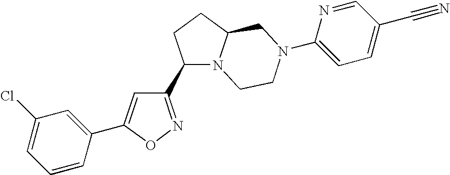 | (±)-6-[(6R,8aS)-6-[5-(3-chlorophenyl)isoxazol-3-yl]hexahydropyrrolo[1,2-a]pyrazin-2(1H)-yl]nicotinonitrile | 66% |
| NMR | 1.75 (m, 1 H), 1.83-2.40 (m, 5 H), 2.78-3.14 (m, 3 H), 3.62 (dd, 1 H), 4.24 (broad d, | | |
| | 1 H), −4.65 (broad d, 1 H), 6.63 (s, 1 H), 6.64 (d, 1 H), 7.42 (m, 2 H), 7.61-7.79 (m, 3 H), | | |
| | 8.42 (s, 1 H). | | |
| 6.3 | 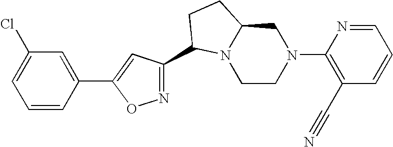 | (±)-2-[(6R,8aS)-6-[5-(3-chlorophenyl)isoxazol-3-yl]hexahydropyrrolo[1,2-a]pyrazin-2(1H)-yl]nicotinonitrile | 36% |
| NMR | 1.68 (m, 1 H), 1.78-2.50 (m, 5 H), 2.94-3.18 (m, 3 H), 3.65 (dd, 1 H), 4.35-4.60 (m, | | |
| | 2 H), 6.63 (s, 1 H), 6.77 (m, 1 H), 7.43 (m, 2 H), 7.69-7.81 (m, 3 H), 8.36 (m, 1 H). | | |
| 6.4 | 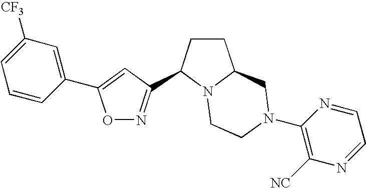 | (±)-3-[(6R,8aS)-6-{5-[3-(trifluoromethyl)phenyl]isoxazol-3-yl}hexahydropyrrolo[1,2-a]pyrazin-2(1H)-yl]pyrazine-2-carbonitrile | 56% |
| NMR | 1.68 (m, 1 H), 1.84-2.08 (m, 2 H), 2.22-2.51 (m, 3 H), 2.92-3.25 (m, 2 H), 3.16-3.25 | | |
| | (m, 1 H), 3.66 (dd, 1 H), 4.50 (m, 1 H), 4.69 (m, 1 H), 6.69 (s, 1 H), 7.62 (dd, 1 H), 7.70 | | |
| | (d, 1 H), 7.97-8.06 (m, 3 H), 8.26 (d, 1 H). | | |
| 6.5 | 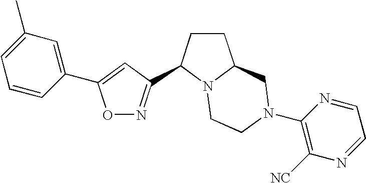 | (±)-3-[(6R,8aS)-6-[5-(3-methylphenyl)isoxazol-3-yl]hexahydropyrrolo[1,2-a}pyrazin-2(1H)-yl)pyrazine-2-carbonitrile | 61% |
| NMR | 1.71 (m, 1 H), 1.86-2.18 (m, 2 H), 2.24-2.53 (m, 3 H), 2.98-3.10 (m, 2 H), 3.17-3.24 | | |
| | (m, 1 H), 3.65 (dd, 1 H), 4.52 (m, 1 H), 4.70 (m, 1 H), 6.59 (s, 1 H), 7.27 (d, 1 H), 7.38 | | |
| | (dd, 1 H), 7.64 (m, 2 H), 8.03 (d, 1 H), 8.27 (d, 1 H). | | |
| 6.6 | 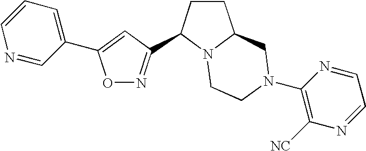 | (±)-3-[6R,8aS)-6-(5-pyridin-3-ylisoxazol-3-yl)hexahydropyrrolo[1,2-a]pyrazin-2(1H)-yl]pyrazine-2-carbonitrile | 25% |
| NMR | 1.71 (m, 1 H), 1.92 (m, 1 H), 2.04 (m, 1 H), 2.25-2.52 (m, 3 H), 2.97-3.07 (m, 2 H), | | |
| | 3.21 (m, 1 H), 3.62 (dd, 1 H), 4.51 (m, 1 H), 4.69 (m, 1 H), 6.69 (s, 1 H), 7.44 (m, 1 H), | | |
| | 8.02 (d, 1 H), 8.11 (m, 1 H), 8.26 (d, 1 H), 8.69 (m, 1 H), 9.03 (s, 1 H). | | |
| 6.7 | 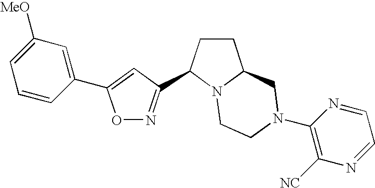 | (±)-3-[(6R,8aS)-6-[5-(3-methoxyphenyl)isoxazol-3-yl]hexahydropyrrolo[1,2-a]pyrazin-2(1H)-yl]pyrazine-2-carbonitrile | 62% |
| NMR | 1.69 (m, 1 H), 1.92 (m, 1 H), 2.02 (m, 1 H), 2.24-2.52 (m, 3 H), 3.00-3.06 (m, 2 H), | | |
| | 3.21 (m, 1 H), 3.63 (dd, 1 H), 3.89 (s, 3 H), 4.50 (m, 1 H), 4.67 (m, 1 H), 6.57 (s, 1 H), | | |
| | 6.99 (m, 1 H), 7.34 (m, 1 H), 7.39 (m, 2 H), 8.01 (d, 1 H), 8.25 (d, 1 H). | | |
| 6.8 | 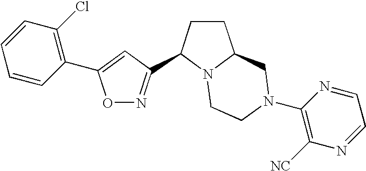 | (±)-3-[6R,8aS)-6-[5-(2-chlorophenyl)isoxazol-3-yl]hexahydropyrrolo[1,2-a]pyrazin-2(1H)-yl]pyrazine-2-carbonitrile | 47% |
| NMR | 1.64-1.76 (m, 1 H), 1.90-2.06 (m, 2 H), 2.24-2.39 (m, 2 H), 2.26 (m, 1 H), 3.00-3.07 | | |
| | (m, 2 H), 3.22 (m, 1 H), 3.65 (dd, 1 H), 4.50 (m, 1 H), 4.68 (m, 1 H), 6.97 (s, 1 H), | | |
| | 7.35-7.43 (m, 2 H), 7.52 (m, 1 H), 7.97 (m, 1 H), 8.02 (d, 1 H), 8.25 (d, 1 H). | | |
| 6.9 | 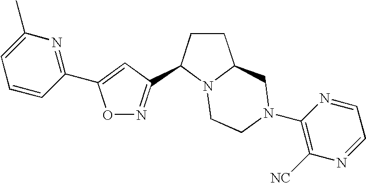 | (±)-3-[(6R,8aS)-6-[5-(6-methylpyridin-2-yl)isoxazol-3-yl]hexahydropyrrolo[1,2-a]pyrazin-2(1H)-yl]pyrazine-2-carbonitrile | 40% |
| NMR | 1.60-1.73 (m, 1 H), 1.88-2.40 (m, 2 H), 2.22-2.30 (m, 1 H), 2.35 (m, 1 H), 2.46 (m, | | |
| | 1 H), 2.62 (s, 3 H), 2.97-3.02 (m, 2 H), 3.19 (m, 1 H), 3.66 (dd, 1 H), 4.49 (m, 1 H), 4.67 | | |
| | (m, 1 H), 6.97 (s, 1 H), 7.19-7.22 (m, 1 H), 7.70 (m, 2 H), 8.01 (d, 1 H), 8.24 (d, 1 H). | | |
| 6.10 | 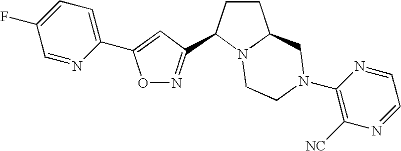 | (±)-3-[6R,8aS)-6-[5-(5-fluoropyridin-2-yl)isoxazol-3-yl]hexahydropyrrolo[1,2-a]pyrazin-2(1H)-yl]pyrazine-2-carbonitrile | 26% |

| | |
|---|---|
| NMR | 1.62-1.73 (m, 1 H), 1.86-2.28 (m, 2 H), 2.30-2.41 (m, 2 H), 2.46 (m, 1 H), 2.96-3.03 |
| | (m, 2 H), 3.19 (m, 1 H), 3.67 (dd, 1 H), 4.49 (m, 1 H), 4.67 (m, 1 H), 6.92 (s, 1 H), 7.55 |
| | (m, 1 H), 7.94 (dd, 1 H), 8.01 (d, 1 H), 8.25 (d, 1 H), 8.55 (d, 1 H). |

### Example 7.1

### (±)-Ethyl (6R,8aS)-6-[5-(3-chlorophenyl)isoxazol-3-yl]hexahydropyrrolo[1,2-a]pyrazine-2(1H)-carboxylate

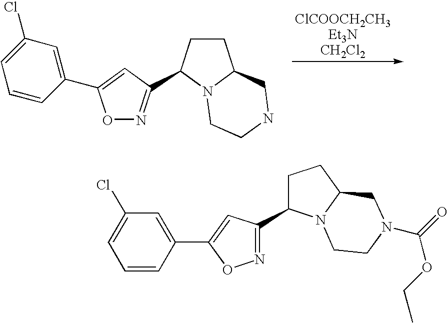

To a mixture of (±)-(6R,8aS)-6-[5-(3-chlorophenyl)isoxazol-3-yl]octahydropyrrolo[1,2-a]pyrazine (40 mg, 0.13 mmol), Et₃N (0.1 mL) and DCM (1 mL) was added ethylchloroformate (30 mg, 0.28 mmol) at −78° C. The mixture was stirred at room temperature for 1 h and was directly subjected to silica gel column to give product (25 mg, 52%). ¹H NMR (300 MHz, CDCl₃): δ (ppm) 1.26 (t, 3H), 1.6 (m, 1H), 1.83-2.3 (m, 5H), 2.68-2.98 (m, 3H), 3.58 (dd, 1H), 4.15 (q, 2H), 4.0-4.4 (m, 2H), 6.59 (s, 1H), 7.43 (m, 2H), 7.69 (m, 1H), 7.79 (s, 1H).

### Example 8.1

### 3-[(6R,8aS)-6-[3-(3-chlorophenyl)isoxazol-5-yl]hexahydropyrrolo[1,2-a]pyrazin-2(1H)-yl]pyrazine-2-carbonitrile

### (i) (±)-3-[(6R,8aS)-6-(hydroxymethyl)hexahydropyrrolo[1,2-a]pyrazin-2(1H)-yl]pyrazine-2-carbonitrile

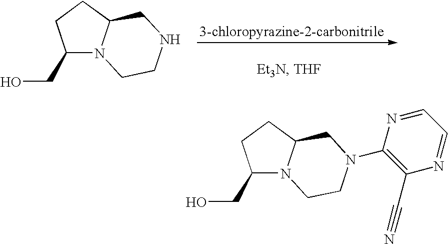

A mixture of (±)-(6R,8aS)-octahydropyrrolo[1,2-a]pyrazin-6-ylmethanol (1.05 g, crude), 3-chloropyrazine-2-carbonitrile (860 mg, 6.2 mmol), Et₃N (1.5 mL) and THF (10 μmL) was stirred at 80° C. overnight. After concentration, the crude product was directly subjected to silica gel column to afford pure product (1.03 g, 77%). ¹H NMR (300 MHz, CDCl₃): δ (ppm) 1.45 (m, 1H), 1.85 (m, 3H), 2.41 (m, 3H), 2.63 (m, 1H), 2.92 (dd, 1H), 3.18 (m, 2H), 3.53 (t, 1H), 3.79 (dd, 1H), 4.61 (m, 2H), 8.03 (s, 1H), 8.27 (s, 1H).

### ii) (±)-3-[(6R,8aS)-6-ethynyl hexahydropyrrolo[1,2-a]pyrazin-2(1H)-yl]pyrazine-2-carbonitrile

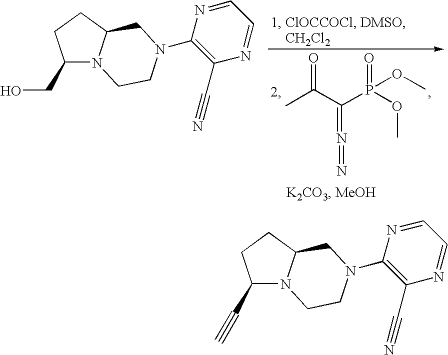

In a similar manner to the procedure described for the boc protected alcohol in example 1.1 iv), the title compound was synthesized from (±)-3-[(6R,8aS)-6-(hydroxymethyl)hexahydropyrrolo[1,2-a]pyrazin-2(1H)-yl]pyrazine-2-carbonitrile in 53% yield. ¹H NMR (300 MHz, CDCl₃): δ (ppm): 1.58 (m, 1H), 1.92 (m, 2H), 2.23 (m, 3H), 2.35 (s, 1H), 2.95 (m, 2H), 3.33 (m, 2H), 4.53 (m, 2H), 7.98 (s, 1H), 8.23 (s, 1H).

### iii) 3-chlorobenzaldehyde oxime

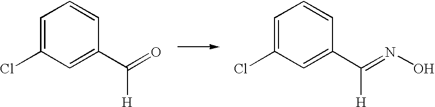

A mixture of 3-chlorobenzaldehyde (1.5 g, 10.67 mmol), hydroxylamine hydrochloride (1.48 g, 21.34 mmol) and sodium acetate (875 mg, 10.67 mmol) in EtOH (16 ml) was stirred at room temperature overnight. The reaction mixture was concentrated in vacuo, and ether was added to the residue. Solids were removed by filtration and the ethereal solution was concentrated in vacuo to afford the title compound (1.75 g) which was used without further purification. ¹H NMR (300 MHz, CDCl₃): δ (ppm) 8.75 (br s, 1H), 8.14 (s, 1H), 7.60 (m, 1H), 7.46 (dm, 1H), 7.37 (m, 2H).

### iv) 3-chloro-N-hydroxybenzenecarboximidoyl chloride

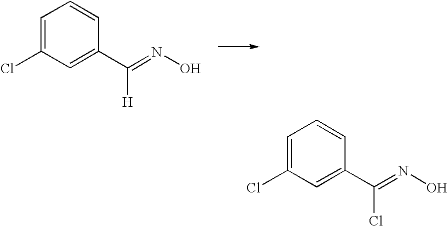

DMF (20 mL) was added to a mixture of N-chlorosuccinimide (1.424 g, 10.67 mmol) and 3-chlorobenzaldehyde oxime (1.66 g, 10.67 mmol) and the resulting mixture was heated at 40° C. for 1 h. The reaction mixture was diluted with diethyl ether and then washed with water, dried, filtered, and concentrated in vacuo to afford the titled compound (1.89 g, 93%, white solid). ¹H NMR (300 MHz, CDCl₃): δ (ppm) 8.62 (br s, 1H), 7.86 (m, 1H), 7.75 (dm, 1H), 7.44 (dm, 1H), 7.36 (t, 1H).

### v) (±)-3-[(6R,8aS)-6-[3-(3-chlorophenyl)isoxazol-5-yl]hexahydropyrrolo[1,2-a]pyrazin-2(1H)-yl]pyrazine-2-carbonitrile

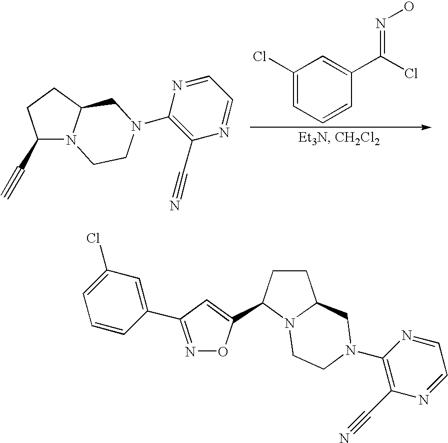

To a solution of 3-chloro-N-hydroxybenzenecarboximidoyl chloride (190 mg, 1 mmol) in DCM (2 mL) was added Et₃N followed by a solution (±)-3-[(6R,8aS)-6-ethynylhexahydropyrrolo[1,2-a]pyrazin-2(1H)-yl]pyrazine-2-carbonitrile (115 mg, 0.45 mmol) in DCM (1.5 mL). The mixture was heated to 68° C. After stirring for 2 h, the resulting mixture was washed with Na₂CO₃ (aq). The organic phase was separated, concentrated and purified on silica gel column to give product (991 mg, 50%). ¹H NMR (300 MHz, CDCl₃): δ (ppm) 1.7 (m, 1H), 2.0-2.5 (m, 5H), 3.00-3.26 (m, 3H), 3.68 (dd, 1H), 4.5-4.7 (m, 2H), 6.53 (s, 1H), 7.43 (m, 2H), 7.71 (m, 1H), 7.83 (s, 1H), 8.04 (d, 1H), 8.28 (d, 1H).

### Example 9.1

### (±)-6-[(6R,9aS)-6-[5-(3-chlorophenyl)isoxazol-3-yl]octahydro-2H-pyrido[1,2-a]pyrazin-2-yl]pyrazine-2-carbonitrile

### i) (±)-Dimethyl (2R,6S)-piperidine-2,6-dicarboxylate hydrochloride

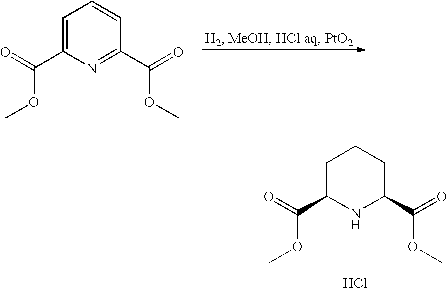

Dimethyl pyridine-2,6-dicarboxylate (15 g, 77 mmol) was dissolved in MeOH (150 mL) and 1 M HCl(aq) (77 mL). The reaction vessel was evacuated of air and backfilled with hydrogen gas, and stirred for 5 days under balloon of hydrogen. When the reaction was complete, the mixture was filtered and concentrated, then dissolved in DCM and washed with Na₂CO₃ (aq). The organic phase was dried, filtered and concentrated to yield the title compound (13.81 g, 89%). ¹H NMR (300 MHz, CDCl₃): δ (ppm) 1.43 (m, 3H); 2.01 (m, 3H); 3.39 (dd, 2H), 3.75 (s, 6H).

### ii) (±)-Methyl (6R,9aS)-1,4-dioxooctahydro-2H-pyrido[1,2-a]pyrazine-6-carboxylate

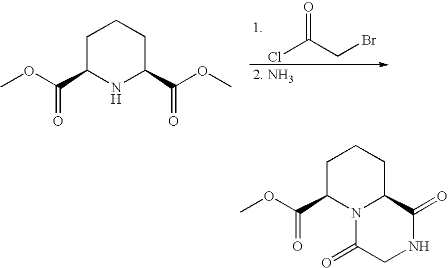

(±)-Dimethyl (2R,6S)-piperidine-2,6-dicarboxylate (7 g, 34.8 mmol), and Na₂CO₃ (7.37 g, 69.5 mmol) were added to a round-bottom flask and dissolved in CH₃CN (50 mL and THF (25 mL). The reaction was cooled to 0° C. and bromoacetyl chloride (6.56 g, 41.7 mmol) was added dropwise. The reaction was stirred until the starting material was no longer observed. The solvent was removed in vacuo, and the residue was dissolved in MeOH (40 mL). The solution was cooled to 0° C. and concentrated ammonia (20 mL) was added. When the intermediate was consumed, the solvent was removed, and the residue was dissolved in DCM and washed with water. The aqueous phase was extracted again with ethyl acetate and added to the DCM. The organic phase was dried filtered and concentrated, then purified by column chromatography to yield the title compound (6.5 g, 83%) ¹H NMR (300 MHz, CDCl₃): δ (ppm) 1.66 (m, 3H); 1.93 (m, 3H); 3.75 (s, 3H); 4.04 (m, 4H); 7.15 (s, broad, 1H).

### iii) (±)-(6R,9aS)-octahydro-2H-pyrido[1,2-a]pyrazin-6-ylmethanol

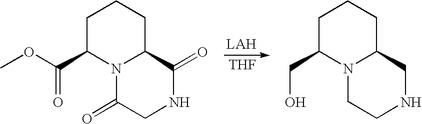

LiAlH₄ (5.45 g, 143 mmol) was added to a three-neck round bottom flask which was purged with argon. THF (250 mL) was added and cooled to 0° C. (±)-Methyl (6R,9aS)-1,4-dioxooctahydro-2H-pyrido[1,2-a]pyrazine-6-carboxylate (6.5 g, 28.7 mmol) was added as a solid, and the reaction was stirred at 40° C. overnight. The reaction was then cooled to 0° C. and quenched with water, slowly. The mixture was filtered through Celite® and washed with ether and ethyl acetate. The filtrate was evaporated to yield the title compound in quantitative yield (4.87 g). ¹H NMR (300 MHz, CDCl₃): δ (ppm) 1.15 (m, 1H); 1.42 (m, 1H); 1.66 (m, 2H); 1.71 (m, 1H); 2.02-2.07 (m, 4H); 2.53 (dd, 1H); 2.85 (t, 2H); 2.99 (m, 2H); 3.12 (m, 1H); 3.36 (dd, 1H); 3.88 (dd, 1H).

### iv) (±)-3-[(6R,9aS)-6-(hydroxymethyl)octahydro-2H-pyrido[1,2-a]pyrazin-2-yl]pyrazine-2-carbonitrile

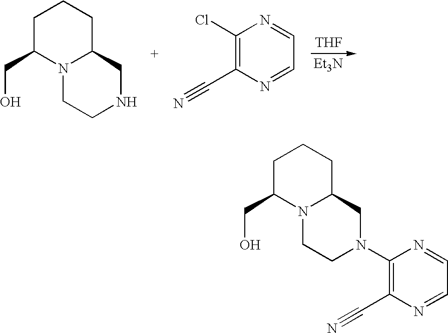

(±)-(6R,9aS)-Octahydro-2H-pyrido[1,2-a]pyrazin-6-ylmethanol (500 mg, 2.93 mmol) was dissolved in THF (7 mL) and Et₃N (2.03 mL, 14.7 mmol). 3-Chloropyrazine-2-carbonitrile (573 mg, 4.11 mmol) was added with stirring, and the reaction was stirred overnight at 35° C. The reaction mixture was then diluted with DCM and washed with water. The organic phase was purified by column chromatography to yield the desired product (650 mg, 81%). ¹H NMR (300 MHz, CDCl₃): δ (ppm) 1.40 (m, 1H); 1.69 (m, 4H); 1.82 (m, 1H); 2.21-2.32 (m, 4H); 2.93 (dd, 1H); 3.28 (m, 2H); 3.35 (d, 1H); 3.96 (dd, 1H); 4.34 (d, 1H); 4.48 (d, 1H); 8.02 (d, 1H); 8.26 (d, 1H).

In a similar manner the following compound was synthesized:

| Ex- | | | |
|---|---|---|---|
| ample | Structure | Name | Yield |
| 9.2 iv | 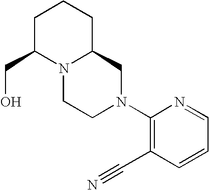 | (±)-2-[(6R,9aS)-6-(hydroxymethyl)octahydro-2H-pyrido[1,2-a]pyrazin-2-yl]nicotinonitrile | 32% |

| | |
|---|---|
| NMR | 1.19-1.43 (m, 2 H); 1.64-1.72 (m, 3 H); 1.80 (m, 3 H); |
| | 2.19 (m, 1 H); 2.24 (m, 1 H); 2.34 (m, 1 H); 2.88 (m, 1 H); |
| | 3.20 (t, 1 H); 3.25 (d, 1 H); 3.39 (d, 1 H); 3.96 (dd, 1 H); |
| | 4.21 (d, 1 H); 4.37 (m, 1 H); 6.76 (dd, 1 H); 7.78 (dd, 1 H); |
| | 8.35 (dd, 1 H) |

### v) (±)-3-[(6R,9aS)-6-formyloctahydro-2H-pyrido[1,2-a]pyrazin-2-yl]pyrazine-2-carbonitrile

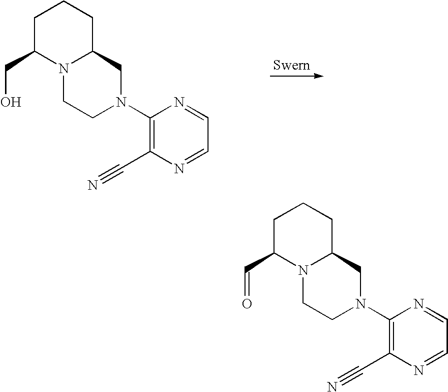

(COCl)₂ (3.99 mmol) was dissolved in DCM (6 mL) and cooled to −78 C. DMSO (5.99 mmol) was added dropwise and stirred for 30 min. (±)-3-[(6R,9aS)-6-(hydroxymethyl)octahydro-2H-pyrido[1,2-a]pyrazin-2-yl]pyrazine-2-carbonitrile (1.99 mmol) was dissolved in DCM (2 mL) and added to the reaction slowly, then stirred at −78 C for 1 h. Et₃N was added and the reaction mixture was allowed to warm to room temperature. The mixture was diluted with DCM and washed with 10% aqueous ammonia. The organic phase was dried, filtered and concentrated to yield the crude title aldehyde.

In a similar manner the following compound was synthesized:

| Example | Structure | Name | Yield |
|---|---|---|---|
| 9.2 v | 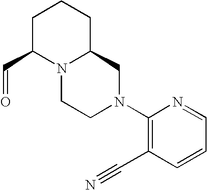 | (±)-2-[(6R,9aS)-6-formyloctahydro-2H-pyrido[1,2-a]pyrazin-2-yl]nicotinonitrile | ND |

### vi) (±)-3-[(6R,9aS)-6-ethynyloctahydro-2H-pyrido[1,2-a]pyrazin-2-yl]pyrazine-2-carbonitrile

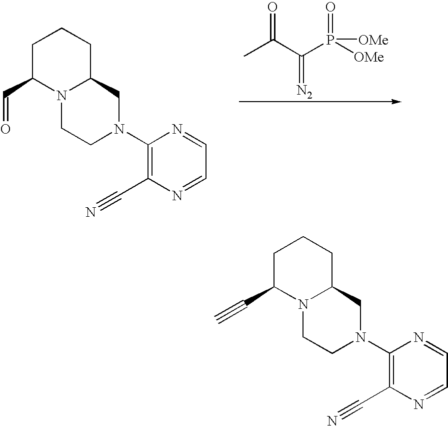

The crude aldehyde was dissolved in MeOH (20 mL). Potassium carbonate (3.99 mmol) was added followed by dimethyl (1-diazo-2-oxopropyl)phosphonate, and the reaction was stirred for 1 h. The solvent was evaporated in vacuo, and the residue was dissolved in DCM and washed with water. The organic phase was purified by column chromatography to yield the title compound (40% over 2 steps). ¹H NMR (300 MHz, CDCl₃): δ (ppm) 1.33 (m, 2H); 1.70 (m, 1H); 1.76 (m, 2H); 1.97 (m, 1H); 2.09-2.18 (m, 2H); 2.34 (d, 1H); 2.80 (d, 1H); 2.89 (t, 1H); 3.26 (td, 1H); 3.64 (d, 1H); 4.27 (d, 1H); 4.44 (d, 1H); 7.94 (d, 1H); 8.20 (d, 1H).

In a similar manner the following compound was synthesized:

| Example | Structure | Name | Yield |
|---|---|---|---|
| 9.2 vi | 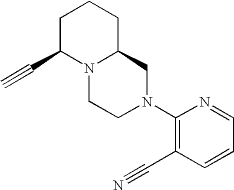 | (±)-2-[(6R,9aS)-6-ethynyloctahydro-2H-pyrido[1,2-a]pyrazin-2-yl]nicotinonitrile | 38% |

| | |
|---|---|
| NMR | 1.26 (m, 2 H); 1.55 (m, 1 H; 1.70 (m, 2 H); 1.95 (m, 1 H); 2.07-2.16 (m, 2 H); |
| | 2.31 (d, 1 H); 2.79 (m, 2 H); 3.16 (td, 1 H); 3.57 (d, 1 H); 4.11 (d, 1 H); 4.32 (d, 1 H); |
| | 6.67 (dd, 1 H); 7.68 (dd, 1 H); 8.25 (dd, 1 H). |

### (vii) 3-[(hydroxyimino)methyl]benzonitrile

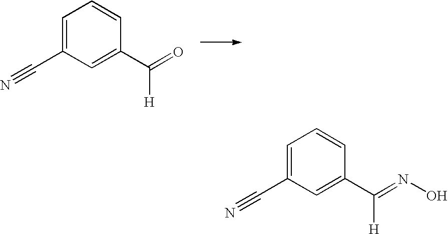

A mixture of 3-formylbenzonitrile (1.399 g, 10.67 mmol), hydroxylamine hydrochloride (1.48 g, 21.34 mmol) and sodium acetate (875 mg, 10.67 mmol) in EtOH (16 ml) was stirred at room temperature for 3 h. The reaction mixture was concentrated in vacuo, and ether was added to the residue. Solids were removed by filtration and the ethereal solution was concentrated in vacuo to afford the title compound (1.54 g, 99%). ¹H NMR (300 MHz, CDCl₃): δ (ppm) 8.16 (s, 1H), 8.05 (br s, 1H), 7.9 (m, 1H), 7.82 (dm, 1H), 7.69 (dm, 1H), 7.55 (t, 1H).

### viii) 3-cyano-N-hydroxybenzenecarboximidoyl chloride

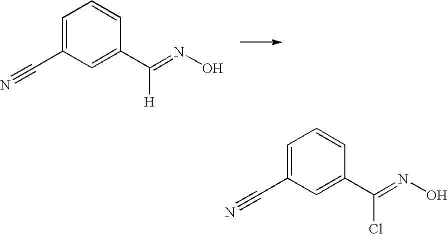

DMF (18 mL) was added to a mixture of N-chlorosuccinimide (1.407 g, 10.54 mmol) and 3-[(hydroxyimino)methyl]benzonitrile (1.54 g, 10.54 mmol) and the resulting mixture was heated at 40° C. for 1 h. The reaction mixture was diluted with diethyl ether and then washed with water, dried, filtered, and concentrated in vacuo to afford the titled compound (1.50 g, 79%). ¹H NMR (300 MHz, CDCl₃): δ (ppm) 8.32 (s, 1H), 8.18 (m, 1H), 8.12 (dm, 1H), 7.75 (dm, 1H), 7.57 (t, 1H).

### ix) (±)-6-[(6R,9aS)-6-[5-(3-chlorophenyl)isoxazol-3-yl]octahydro-2H-pyrido[1,2-a]pyrazin-2-yl]pyrazine-2-carbonitrile

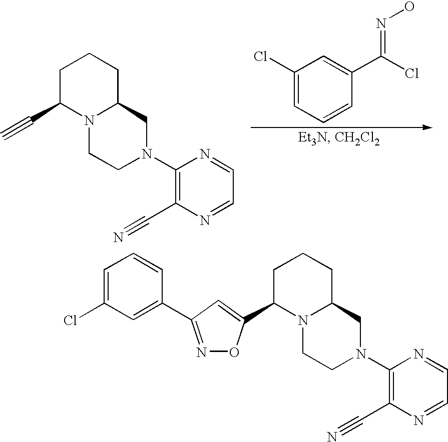

To a solution of 3-chloro-N-hydroxybenzenecarboximidoyl chloride (292 mg, 1.54 mmol) and (±)-3-[(6R,9aS)-6-ethynyloctahydro-2H-pyrido[1,2-a]pyrazin-2-yl]pyrazine-2-carbonitrile (274.2 mg, 1.02 mmol) in DCM (9.0 mL) at 0° C. was added Et₃N (200 μL). The mixture stirred overnight at room temperature. The resulting mixture was washed with water. The organic phase was separated, concentrated and purified on silica gel column to give product (119 mg, 26%). ¹H NMR (300 MHz, CDCl₃): δ (ppm) 1.48 (m, 2H), 1.75 (m, 1H); 1.90 (m, 3H); 2.22-2.37 (m, 2H); 2.75 (d, 1H); 2.98 (dd, 1H); 3.19 (td, 1H); 3.42 (dd, 1H); 4.35 (d, 2H); 6.52 (s, 1H); 7.36 (m, 2H); 7.67 (m, 1H); 7.77 (m, 1H); 7.97 (d, 1H); 8.22 (d, 1H).

In a similar manner the following compounds were synthesized:

| Example | Structure | Name | Yield |
|---|---|---|---|
| 9.2 | 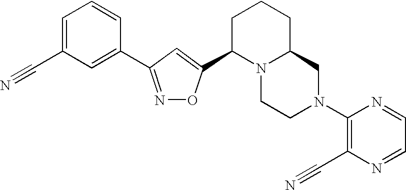 | (±)-6-[(6R,9aS)-6-[5-(3-cyanophenyl)isoxazol-3-yl]octahydro-2H-pyrido[1,2-a]pyrazin-2-yl]pyrazine-2-carbonitrile | 36% |
| NMR | 1.48 (m, 2 H), 1.75 (m, 1 H); 1.90 (mm, 3 H); 2.22-2.37 (m, 2 H); 2.75 (d, 1 H); 2.98 (dd, | | |
| | 1 H); 3.19 (td, 1 H); 3.42 (dd, 1 H); 4.35 (d, 2 H); 6.57 (s, 1H); 7.56 (t, 1 H); 7.69 (d, 1 H); | | |
| | 7.96 (d, 1 H); 8.01-8.05 (m, 2 H); 8.21 (d, 1 H) | | |
| 9.3 | 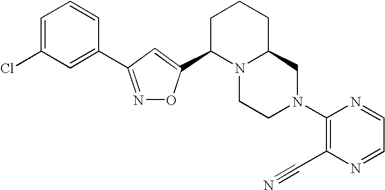 | (±)-2-[(6R,9aS)-6-[3-(3-chlorophenyl)isoxazol-5-yl]octahydro-2H-pyrido[1,2-a]pyrazin-2-yl]nicotinonitrile | 57% |
| NMR | 8.33 (dd, 1 H), 7813 (m, 1 H), 7.76 (dd, 1 H), 7.69 (m, 1 H), 7.4 (m, 2 H), 6.75 (q, 1 H), 6.54 | | |
| | (s, 1 H), 4.26 (m, 2 H), 3.44 (m, 1 H), 3.17 (m, 1 H), 2.96 (m, 1 H), 2.76 (m, 1 H), 2.33 (m, | | |
| | 2 H), 1.92 (m, 3 H), 1.7 (m, 1 H), 1.51 (m, 2 H) | | |
| 9.4 | 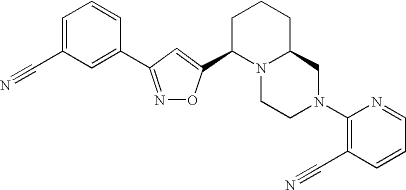 | (±)-2-[(6R,9aS)-6-[3-(3-cyanophenyl)isoxazol-5-yl]octahydro-2H-pyrido[1,2-a]pyrazin-2-yl]nicotinonitrile | 30% |

| | |
|---|---|
| NMR | 8.34 (dd, 1 H), 8.08 (m, 2 H), 7.75 (m, 2 H), 7.6 (t, 1 H), 6.76 (q, 1 H), 6.58 (s, 1 H), 4.27 (m, |
| | 2 H), 3.48 (m, 1 H), 3.18 (m, 1 H), 2.96 (m, 1 H), 2.75 (m, 1 H), 2.35 (m, 2 H), 1.95 (m, 3 H), |
| | 1.75 (m, 1 H), 1.52 (m, 2 H) |

### Example 10.1

### (±)-2-[(6R,8aS)-6-[1-(3-chlorophenyl)-1H-1,2,3-triazol-4-yl]hexahydropyrrolo[1,2-a]pyrazin-2(1H)-yl]-5-fluoronicotinonitrile

### (i) 2-Chloro-5-fluoronicotinonitrile

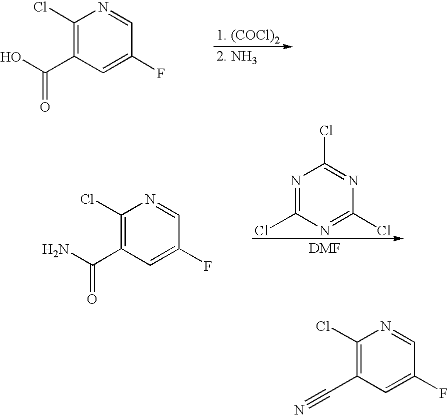

2-Chloro-5-fluoronicotinic acid (1.28 g, 7.3 mmol) was dissolved in DCM (18 mL) and DMF (4 drops). (COCl)₂ (7.3 mL, 2M in DCM) was added and the reaction was stirred for 1 h. Solvent and excess (COCl)₂ was removed in vacuo and the residue was dissolved in THF (12 mL). Concentrated ammonium hydroxide (4 mL) was added and stirred until the reaction was complete. The reaction mixture was diluted with diethyl ether and washed with water. The organic phase was dried, filtered and concentrated, then dissolved in DMF. Trichlorotriazine (808 mg, 4.38 mmol) was added in four portions, 20 min apart. When the reaction was complete, the mixture was diluted with diethyl ether and washed with water. The organic phase was dried, filtered and concentrated to yield the title compound in quantitative yield (1.13 g).

### (ii) 5-Fluoro-2-[(6R,9aS)-6-(hydroxymethyl)hexahydropyrrolo[1,2-a]pyrazin-2(1H)-yl]nicotinonitrile

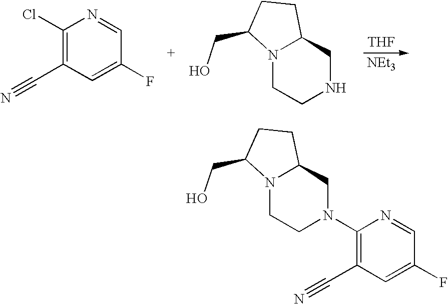

(±)-(6R,8aS)-Octahydropyrrolo[1,2-a]pyrazin-6-ylmethanol (800 mg, 4.69 mmol) and 2-chloro-5-fluoronicotinonitrile (809.1 mg, 5.16 mmol) were dissolved in THF (12 mL). Et₃N (3.27 mL, 23.5 mmol) was added and the reaction was stirred for 3 days at 35° C. The reaction mixture was diluted with DCM and washed with water. The organic phase was purified by chromatography on silica gel using ethyl acetate to yield the title compound (600 mg, 43%).

### (iii) 2-[(6R,9aS)-6-ethynylhexahydropyrrolo[1,2-a]pyrazin-2-yl-5-fluoronicotinonitrile

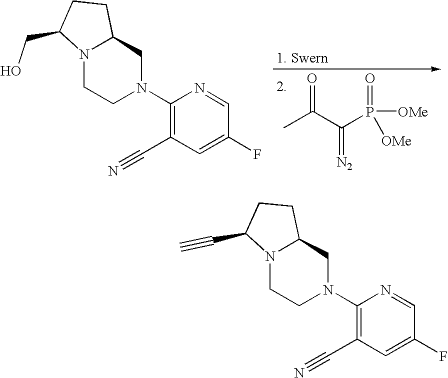

(COCl)₂ (4.34 mL, 8.68 mmol, 2 M in DCM) was dissolved in DCM (12.2 mL) and cooled to −78° C. DMSO (0.916 mL, 13.02 mmol) was added dropwise and stirred for 30 min. 5-Fluoro-2-[(6R,9aS)-6-(hydroxymethyl)hexahydropyrrolo[1,2-a]pyrazin-2(1H)-yl]nicotinonitrile (1.2 g, 4.34 mmol) in DCM (3 mL) was added slowly and the reaction mixture was stirred for 1 h. Et₃N (2.4 mL) was added and the reaction was stirred at room temperature. The mixture was diluted with DCM and washed with 10% NH₃ (aq). The organic phase was dried, filtered and concentrated. The residue was dissolved in MeOH (30 mL) and potassium carbonate (1.19 g, 8.68 mmol) was added, followed by dimethyl (1-diazo-2-oxopropyl)phosphonate (1.0 g, 5.21 mmol). The reaction was stirred at room temperature for 1 h. The resulting mixture was concentrated in vacuo, dissolved in dichloromethane and washed with water. The organic phase was dried filtered and concentrated, then purified by column chromatography to yield the title compound (449 mg, 38%).

### (iv) (±)-2-[(6R,8aS)-6-[1-(3-chlorophenyl)-1H-1,2,3-triazol-4-yl]hexahydropyrrolo[1,2-a]pyrazin-2(1H)-yl]-5-fluoronicotinonitrile

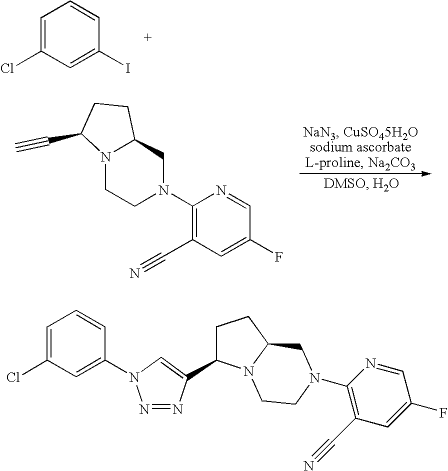

(±)-2-[(6R,9aS)-6-ethynylhexahydropyrrolo[1,2-a]pyrazin-2-yl-5-fluoronicotinonitrile (81.1 mg, 0.30 mmol), 1-chloro-3-iodobenzene (71.5 mg, 0.30 mmol), sodium azide (23.4 mg, 0.36 mmol) copper (II) sulfate pentahydrate (3.7 mg, 0.015 mmol), sodium ascorbate (5.2 mg, 0.03 mmol), L-proline (6.9 mg, 0.06 mmol) and Na₂CO₃ (6.3 mg, 0.06 mmol) were added to a screw cap vial with a stir bar and dissolved in DMSO (0.9 mL). Water (0.1 mL) was added and the reaction was stirred at 68° C. overnight. The mixture was then diluted with DCM and washed with water. The organic phase was purified via a silica gel column to yield the title compound (72.9 mg, 57%). ¹H NMR (300 MHz, CDCl₃): δ (ppm) 1.64 (m, 1H); 1.94-1.99 (m, 2H); 2.21-2.59 (m, 3H); 2.95 (dd, 1H); 3.04 (d, 1H); 3.15 (td, 1H); 3.71 (t, 1H); 4.20 (d, 1H); 4.36 (d, 1H); 7.42 (m, 2H); 7.53 (m, 1H); 7.67 (m, 1H); 7.80 (m, 1H); 7.95 (s, 1H); 8.24 (d, 1H).

In a similar manner the following compounds were synthesized:

| Example | Structure | Name | Yield |
|---|---|---|---|
| 10.2 | 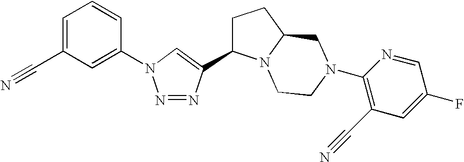 | (±)-6-[(6R,9aS)-6-[5-(3-cyanophenyl)isoxazol-3-yl]hexahydropyrrolo[1,2-a]pyrazin-2-yl]-5-fluoronicotinonitrile | 57% |

| | |
|---|---|
| NMR | 1.64 (m, 1 H); 1.94-1.99 (m, 2 H); 2.21-2.59 (m, 3 H); 2.95 (dd, 1 H); 3.04 (d, 1 H); 3.15 (td, |
| | 1 H); 3.73 (t, 1 H); 4.19 (d, 1 H); 4.37 (d, 1 H); 7.54 (m, 1 H); 7.72 (m, 2 H); 8.01 (m, 1 H); |
| | 8.09 (s, 2 H); 8.25 (d, 1 H). |

### Example 11.1

### (±)-2-[(6R,9aS)-6-[5-(3-chlorophenyl)isoxazol-3-yl]octahydro-2H-pyrido[1,2-a]pyrazin-2(1H)-yl]nicotinonitrile

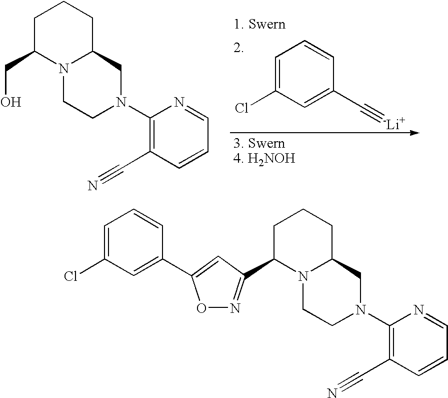

(COCl)₂ (3.67 mmol) was dissolved in DCM and cooled to −78° C. DMSO (5.50 mmol) was added slowly and stirred for 30 min. 2-[6-(hydroxymethyl)octahydro-2H-pyrido[1,2-a]pyrazin-2-yl]benzonitrile (498 mg, 1.83 mmol) was dissolved in DCM (3 mL) and added to the reaction mixture. The reaction was stirred at −78° C. for 1 h then Et₃N was added and the mixture was warmed to room temperature. The mixture was diluted with DCM and washed with 10% NH₃ (aq). The organic phase was purified by column chromatography.

1-Chloro-3-ethynylbenzene (380 mg, 2.77 mmol) was dissolved in THF (5 mL) and cooled to 0° C. n-Butyl lithium (2.5 M) was added slowly and stirred for 20 min. The aldehyde was dissolved in THF and cooled to 0° C., and the acetylide solution was transferred into it, and stirred overnight at room temperature. The mixture was diluted with DCM and washed with water. Both enantiomers of the alcohol were isolated by column chromatography and combined, and oxidized to the ketone via the same method as above. The ketone (0.914 mmol) was stirred in EtOH at room temperature with hydroxylamine hydrochloride (79 mg, 1.14 mmol) and Na₂CO₃ (64 mg, 0.60 mmol). The mixture was stirred for 3 days and diluted with DCM and washed with water. The desired product was isolated in 5% yield by column chromatography. ¹H NMR 300 MHz, (CDCl₃) δ: 1.52 (m, 2H); 1.89 (m, 1H); 1.98 (m, 3H); 2.23-2.38 (m, 2H); 2.78 (d, 1H); 2.99 (td, 1H); 3.18 (td, 1H); 3.47 (d, 1H); 4.28 (d, 2H); 6.54 (s, 1H); 6.77 (dd, 1H); 7.42 (m, 2H); 7.70-7.83 (m, 3H); 8.35 (dd, 1H).

### Example 12.1

### (±)-6-[(6R,9aS)-6-[5-(3-cyanophenyl)-1,2,3-triazol-4-yl]octahydro-2H-pyrido[1,2-a]pyrazin-2-yl]pyrazine-2-carbonitrile

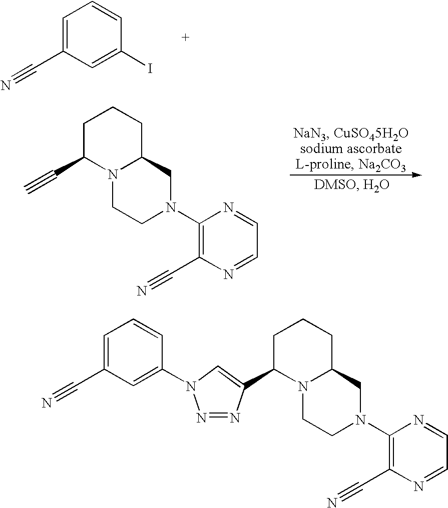

(±)-3-[(6R,9aS)-6-ethynyloctahydro-2H-pyrido[1,2-a]pyrazin-2-yl]pyrazine-2-carbonitrile (80 mg, 0.3 mmol), 3-iodobenzonitrile (69 mg, 0.3 mmol), sodium azide (23 mg, 0.36 mmol) copper (II) sulfate pentahydrate (3.7 mg, 0.015 mmol), sodium ascorbate (6 mg, 0.03 mmol), L-proline (7 mg, 0.06 mmol) and Na₂CO₃ (6.4 mg, 0.06 mmol) were added to a screw cap vial with a stirring bar and dissolved in DMSO (0.9 mL). Water (0.1 mL) was added and the reaction was stirred at 68° C. overnight. The mixture was then diluted with DCM and washed with water. The organic phase was purified via a silica gel column to yield the desired product (53%). ¹H NMR 300 MHz, (CDCl₃) δ: 1.53 (m, 2H); 1.76 (m, 1H); 1.89 (m, 3H); 2.24 (m, 1H); 2.37 (m, 1H); 2.81 (d, 1H); 3.00 (t, 1H); 3.18 (t, 1H); 3.51 (d, 1H); 4.36 (m, 2H); 7.70 (m, 2H); 7.98 (d, 1H); 8.09 (m, 3H); 8.23 (d, 1H).

In a similar manner the following compounds were synthesized:

| Example | Structure | Name | Yield |
|---|---|---|---|
| 12.2 | 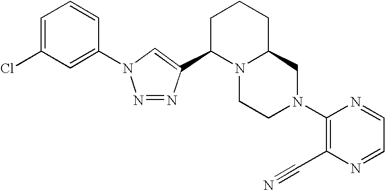 | (±)-[(6R,9aS)-6-[5-(3-chloro-phenyl)-1,2,3-triazol-4-yl]octahydro-2H-pyrido[1,2-a]pyrazin-2-yl]pyrazine-2-carbonitrile | 52% |
| NMR | 1.58 (m, 2 H); 1.8 (m, 4 H); 2.22 (m, 1 H); 2.34 (m, 1 H); 2.82 (d, 1 H); 2.99 (dd, 1 H); 3.17 | | |
| | (dd, 1 H); 3.51 (d, 1 H); 4.37 (m, 2 H); 7.44 (m, 2 H); 7.66 (d, 1 H); 7.80 (m, 1 H); 7.97 (d, | | |
| | 1 H); 8.23 (d, 1 H). | | |
| 12.3 | 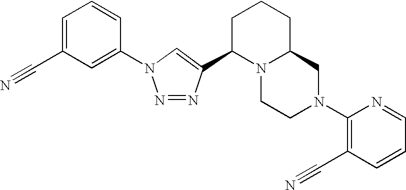 | (±)-6-[(6R,9aS)-6-[5-(3-cyanophenyl)-1,2,3-triazol-4-yl]octahydro-2H-pyrido[1,2-a]pyrazin-2(1H)-yl]nicotinonitrile | 42% |
| NMR | 1.51 (m, 2 H); 1.75 (m, 1 H); 1.88 (m, 3 H); 2.26 (td, 1 H); 2.37 (m, 1 H); 2.78 (d, 1 H); | | |
| | 2.93 (t, 1 H); 3.12 (td, 1 H); 3.51 (d, 1 H); 4.25 (m, 2 H); 6.73 (dd, 1 H); 7.72 (m, 3 H); 8.05 | | |
| | (m, 3 H); 8.31 (dd, 1 H). | | |
| 12.4 | 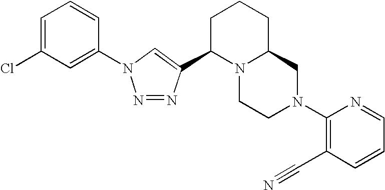 | (±)-6-[(6R,9aS)-6-[5-(3-chloro-phenyl)-1,2,3-triazol-4-yl]octahydro-2H-pyrido[1,2-a]pyrazin-2(1H)-yl]nicotinonitrile | 43% |

| | |
|---|---|
| NMR | 1.50 (m, 2 H); 1.85 (m, 4 H); 2.26 (td, 1 H); 2.37 (m, 1 H); 2.79 (d, 1 H); 2.94 (dd, 1 H); |
| | 3.12 (td, 1 H); 3.49 (d, 1 H); 4.26 (m, 2 H); 6.72 (dd, 1 H); 7.41 (m, 1 H); 7.44 (t, 1 H); 7.66 |
| | (m, 1 H); 7.74 (d, 1 H); 7.81 (dd, 1 H); 7.97 (s, 1 H); 8.32 (dd, 1 H). |

### Example 13.1

### (±)-3-[(6R,9aS)-6-[5-(3-cyanophenyl)isoxazol-3-yl]octahydro-2H-pyrido[1,2-a]pyrazin-2-yl]pyrazine-2-carbonitrile

### (i) 3-Ethynylbenzonitrile

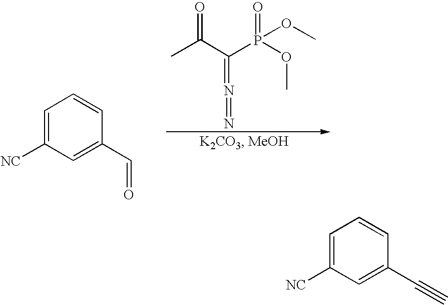

Potassium carbonate (421 mg, 3.0 mmol) and dimethyl (1-diazo-2-oxopropyl)phosphonate (351 mg, 1.83 mmol) were sequentially added to a solution of 3-formylbenzonitrile (200 mg, 1.52 mmol) in MeOH (10 mL) at room temperature. After stirring at room temperature for 1 h, the resulting mixture was concentrated. The residue was dissolved in DCM and the organic layer was washed with water. After removal of the solvent, flash chromatography on silica gel afforded acetylene (94.6 mg, 49%). ¹H NMR 300 MHz, (CDCl₃) δ (ppm) 7.73 (m, 1H), 7.68 (dd, 1H), 7.62 (dd, 1H), 7.45 (t, 1H), 3.22 (s, 1H).

In a similar manner, the following compounds were synthesized:

| Example | Structure | Name | Yield |
|---|---|---|---|
| 13.2 i | 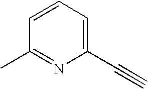 | 2-ethynyl-6-methylpyridine | 68% |

| | |
|---|---|
| NMR | 7.52 (t, 1 H), 7.27 (d, 1 H), 7.1 (d, 1 H), 3.11 (s, 1 H), |
| | 2.53 (s, 3 H) |

### (ii) (±)-3-{(6R,9aS)-6-[(E)-(hydroxyimino)methyl]octahydro-2H-pyrido[1,2-a]pyrazin-2-yl}pyrazine-2-carbonitrile

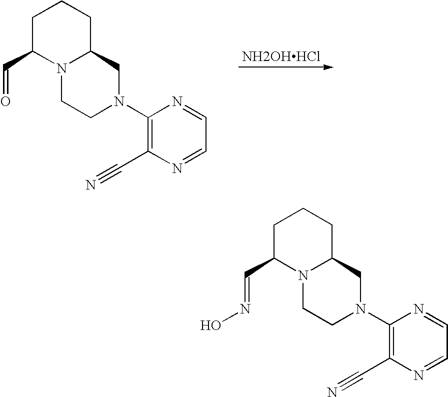

A solution of (±)-3-[(6R,9aS)-6-formyloctahydro-2H-pyrido[1,2-a]pyrazin-2-yl]pyrazine-2-carbonitrile (130 mg, 0.48 mmol) in THF (2 ml) was added to a solution of hydroxylamine hydrochloride (49.9 mg, 0.72 mmol) and sodium acetate (39.3 mg, 0.48 mmol) in EtOH (2 mL) and the resulting mixture was stirred at room temperature for 3 h. Concentration in vacuo and trituration with ethyl acetate afforded the title compound (138 mg, 100%).

### iii) (±)-(6R,9aS)-2-(3-cyanopyrazin-2-yl)-N-hydroxyoctahydro-2H-pyrido[1,2-a]pyrazine-6-carboximidoyl chloride

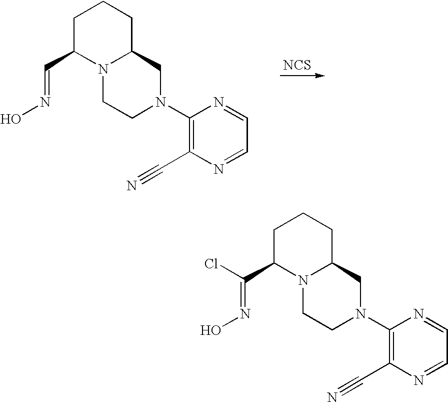

N-chlorosuccinimide (31.7 mg, 0.24 mmol) was added to a solution of (±)-3-{(6R,9aS)-6-[(E)-(hydroxyimino)methyl]octahydro-2H-pyrido[1,2-a]pyrazin-2-yl}pyrazine-2-carbonitrile (68 mg, 0.24 mmol) in HCl (178 μL, 2M in ether) and DMF (1 mL) at room temperature. The resulting solution was heated to 60° C. for 40 min, then used in the subsequent step without any workup or purification.

### iv) (±)-3-[(6R,9aS)-6-[5-(3-cyanophenyl)isoxazol-3-yl]octahydro-2H-pyrido[1,2-a]pyrazin-2-yl]pyrazine-2-carbonitrile

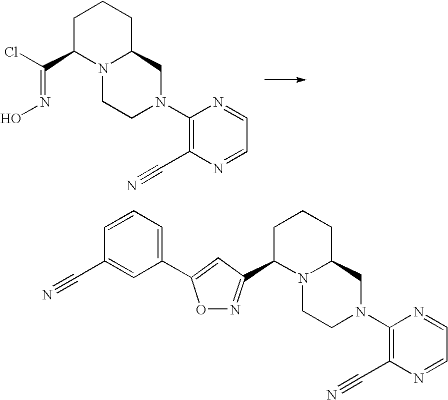

A solution of 3-ethynylbenzonitrile (60 mg, 0.47 mmol) and Et₃N (99.2 μL, 0.71 mmol) in DCM (2 mL) was added to the DMF solution of (±)-(6R,9aS)-2-(3-cyanopyrazin-2-yl)-N-hydroxyoctahydro-2H-pyrido[1,2-a]pyrazine-6-carboximidoyl chloride from above (77 mg, 0.24 mmol). The resulting mixture was stirred at room temperature overnight. Standard work up and purification by chromatography yielded the isoxazole (4.7 mg, 5%). ¹H NMR 300 MHz, (CDCl₃) δ (ppm) 8.23 (d, 1H), 8.02 (m, 3H), 7.73 (dd, 1H), 7.63 (t, 1H), 6.57 (s, 1H), 4.42 (m, 2H), 4.33 (m, 1H), 3.3 (m, 1H), 3.09 (m, 1H), 2.91 (m, 2H), 2.64 (m, 1H), 1.89 (m, 3H), 1.67 (m, 2H), 1.41 (m, 1H).

In a similar manner, the following compounds were synthesized:

| Example | Structure | Name | Yield |
|---|---|---|---|
| 13.2 | 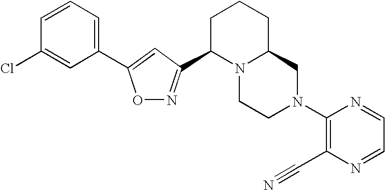 | (±)-3-[(6R,9aS)-6-[5-(3-chlorophenyl)isoxazol-3-yl]octahydro-2H-pyrido[1,2-a]pyrazin-2-yl]pyrazine-2-carbonitrile | 32% |
| NMR | 8.23 (dd, 1 H), 7.98 (dd, 1 H), 7.75 (m, 1 H), 7.65 (m, 1 H), 7.41 (m, 2 H), 6.5 (s, 1 H), 4.41 | | |
| | (m, 2 H), 4.31 (m, 1 H), 3.3 (m, 1 H), 3.06 (m, 1 H), 2.86 (m, 2 H), 2.63 (m, 1 H), 1.87 (m, | | |
| | 3 H), 1.74 (m, 2 H), 1.4 (m, 1 H) | | |
| 13.3 | 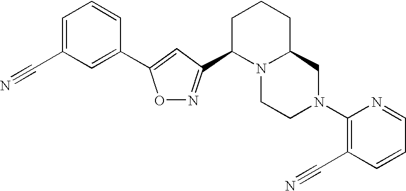 | (±)-2-[(6R,9aS)-6-[5-(3-cyanophenyl)isoxazol-3-yl]octahydro-2H-pyrido[1,2-a]pyrazin-2-yl]nicotinonitrile | 13% |
| NMR | 8.33 (dd, 1 H), 8.05 (m, 2 H), 7.74 (m, 2 H), 7.62 (t, 1 H), 6.73 (q, 1 H), 6.6 (s, 1 H), 4.3 (m, | | |
| | 3 H), 3.25 (m, 1 H), 3.08 (m, 1 H), 2.85 (m, 2 H), 2.64 (m, 1 H), 2.15 (m, 1 H), 1.88 (m, | | |
| | 3 H), 1.66 (m, 1 H), 1.2 (m, 1H) | | |
| 13.4 | 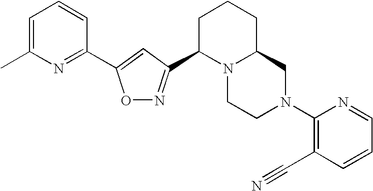 | (±)-2-[(6R,9aS)-6-[5-(6-methylpyrid-2-yl)isoxazol-3-yl]octahydro-2H-pyrido[1,2-a]pyrazin-2-yl]nicotinonitrile | 18% |

| | |
|---|---|
| NMR | 8.31 (dd, 1 H), 7.73 (m, 3 H), 7.2 (m, 1 H), 6.91 (s, 1 H), 6.71 (m, 1 H), 4.32 (m, 3 H), 3.25 |
| | (m, 1 H), 3.1 (m, 1 H), 2.81 (m, 2 H), 2.66 (m, 1 H), 2.62 (s, 3 H), 2.15 (m, 1 H), 1.89 (m, |
| | 3 H), 1.75 (m, 1 H), 1.29 (m, 1 H) |

### Example 14.1

### (±)-6-[(6R,9aS)-6-[5-(pyrid-2-yl)isoxazol-3-yl]octahydro-2H-pyrido[L1,2-a]pyrazin-2-yl]pyrazine-2-carbonitrile

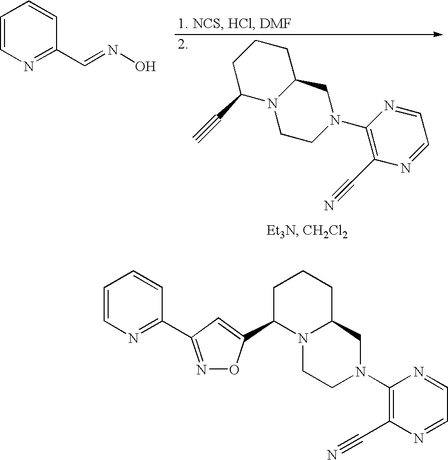

N-chlorosuccinimide (29.4 mg, 0.22 mmol) was added to a solution of pyridine-2-carbaldehyde oxime (26.9 mg, 0.22 mmol) in HCl (275 μL, 2M in ether) and DMF (0.9 mL) at room temperature. The resulting solution was heated to 60° C. for 40 min. A solution of (±)-3-[(6R,9aS)-6-ethynyloctahydro-2H-pyrido[1,2-a]pyrazin-2-yl]pyrazine-2-carbonitrile (49 mg, 0.18 mmol) and Et₃N (76.6 μL, 0.55 mmol) in DCM (2 mL) was added to the DMF solution from above. The resulting mixture was stirred at room temperature overnight. Standard work up and purification by chromatography yielded the isoxazole (10.1 mg, 14%). ¹H NMR 300 MHz, (CDCl₃) δ (ppm) 8.71 (dd, 1H), 8.25 (d, 1H), 8.09 (dd, 1H), 8.01 (d, 1H), 7.82 (m, 1H), 7.37 (m, 1H), 6.89 (s, 1H), 4.38 (m, 2H), 3.48 (m, 1H), 3.24 (m, 1H), 3.04 (m, 1H), 2.83 (m, 1H), 2.31 (m, 2H), 1.95 (m, 3H), 1.77 (m, 1H), 1.53 (m, 2H)

### Example 15.1

### (±)-3-[(6R,8aS)-6-[1-(3-methylphenyl)-1H-1,2,3-triazol-4-yl]hexahydropyrrolo[1,2-a]pyrazin-2(1H)-yl]pyrazine-2-carbonitrile

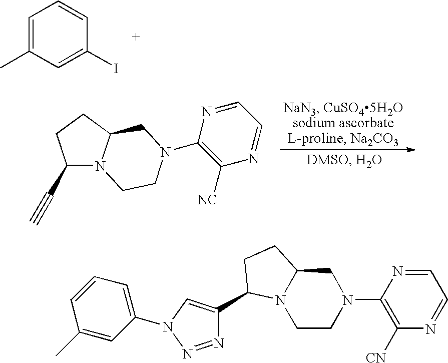

A mixture of 3-(6-ethynylhexahydropyrrolo[1,2-a]pyrazin-2(1H)-yl)pyrazine-2-carbonitrile (100 mg, 0.40 mmol), 3-iodotoluene (103 mg, 0.47 mmol), sodium azide (30 mg, 0.47 mmol), copper sulfate pentahydrate (10 mg, 0.04 mmol), sodium-L-ascorbate (16 mg, 0.08 mmol), L-proline (9 mg, 0.08 mmol), Na₂CO₃ (9 mg, 0.08 mmol), DMSO (1.8 mL) and H₂O (0.2 mL) was stirred at 70° C. for 8 h. The resulting mixture was diluted with ethyl acetate and washed with saturated Na₂CO₃ (aq). The organic phase was dried over sodium sulphate, concentrated and purified by silica gel chromatography to afford the title compound (160 mg, 80%). ¹H NMR 400 MHz, (CDCl₃) δ (ppm) 8.3 (s, 1H), 8.0 (d, 2H), 7.6 (s, 1H), 7.5 (d, 1H), 7.4 (m, 1H), 7.2 (m, 1H), 4.7 (d, 1H), 4.5 (d, 1H), 3.8 (t, 1H), 3.3 (t, 1H), 3.2-3.0 (m, 2H), 2.6-2.3 (m, 6H), 2.1-1.9 (m, 2H), 1.8-1.6 (m, 1H). ESMS: m/s 387.00 [M⁺+1]

In a similar manner the following compounds were synthesized:

| Example | Structure | Name | Yield |
|---|---|---|---|
| 15.2 | 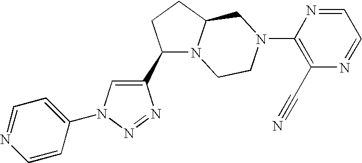 | (±)-3-[(6R,8aS)-6-(1-pyridin-4-yl-1H-1,2,3-triazol-4-1,2-a]pyrazin-yl)hexahydropyrrolo[1,2-a]pyrazin-2(1H)-yl]pyrazine-2-carbonitrile | 68% |
| NMR | 8.8 (s, 2 H), 8.3 (s, 1 H), 8.1 (d, 2 H), 7.8 (m, 2 H), 4.7 (d, 1 H), 4.5 (d, 1 H), 3.8 (t, 1 H), 3.3 | | |
| | (t, 1 H), 3.2-3.0 (m, 2 H), 2.6-2.3 (m, 3 H), 2.1-1.9 (m, 2 H), 1.8-1.6 (m, 1 H). | | |
| 15.3 | 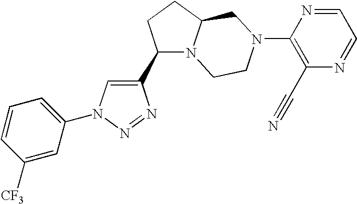 | (±)-3-[6R,8aS)-6-[1-(3-trifluoromethylphenyl)-1H-1,2,3-triazol-4-yl]hexahydropyrrolo[1,2-a]pyrazin-2(1H)-yl]pyrazine-2-carbonitrile | 68% |
| NMR | 8.25 (d, 1 H), 8.1 (m, 4 H), 7.7 (m, 2 H), 4.7 (d, 1 H), 4.5 (d, 1 H), 3.8 (t, 1 H), 3.3 (t, 1 H), | | |
| | 3.2-3.0 (m, 2 H), 2.6-2.3 (m, 3 H), 2.1-1.9 (m, 2 H), 1.8-1.6 (m, 1 H). | | |
| 15.4 | 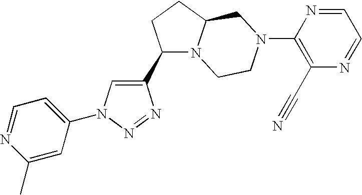 | (±)-3-[(6r,8aS)-6-[1-(2-methylpyridin-4-yl)-1H-1,2,3-triazol-4-yl]hexahydropyrrolo[1,2-a]pyrazin-2(1H)-yl]pyrazine-2-carbonitrile | 56% |

| | |
|---|---|
| NMR | 8.68 (d, 1 H), 8.26 (m, 1 H), 8.02 (t, 2 H), 7.64 (s, 1 H), 7.56 (m, 1 H), 4.68 (d, 1 H), 4.50 (d, |
| | 1 H), 3.74 (t, 1 H), 3.24 (t, 1 H), 3.10-3.02 (m, 2 H), 2.68 (s, 3 H), 2.50-2.30 (m, 3 H), |
| | 2.06-1.92 (m, 2 H), 1.76-1.66 (m, 1 H). |

### Example 16

### Chiral HPLC Separation of Enantiomers

| Example | Structure | Name |
|---|---|---|
| 16.1 | 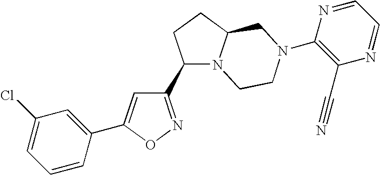 | 3-[(6R,8aS)-6-[5-(3-chlorophenyl)isoxazol-3-yl]hexahydropyrrolo[1,2-a]pyrazin-2(1H)-yl]pyrazine-2-carbonitrile&3-[(6S,8aR)-6-[5-(3-chlorophenyl)isoxazol-3-yl]hexahydropyrrolo[1,2-a]pyrazin-2(1H)-yl]pyrazine-2-carbonitrile |
| HPLC details | Fraction 1: pale-brown foam, 72 mg, Rt 17.03 min | |
| | Fraction 2: pale-brown foam, 74.6 mg, Rt 25.34 min | |
| | (using chiralcel OJ 4.6 X 250 mm column; EtOH/petroleum ether 40/60; 0.7 | |
| | mL/min) at 39° C. | |
| 16.2 | 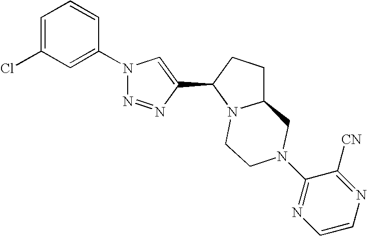 | 3-[(6R,8aS)-6-[1-(3-chlorophenyl)-1H-1,2,3-triazol-4-yl]hexahydropyrrolo[1,2-a]pyrazin-2(1H)-yl]pyrazine-2-carbonitrile&3-[(6S,8aR)-6-[1-(3-chlorophenyl)-1H-1,2,3-triazol-4-yl]hexahydropyrrolo[1,2-a]pyrazin-2(1H)-yl]pyrazine-2-carbonitrile |
| HPLC details | Fraction 1: pale-yellow solid, 78 mg, Rt 13.9 min | |
| | Fraction 2: off-white solid, 79 mg, Rt 23.2 min | |
| | (using Chiralpak AD 21 mm ID × 250 mm, 20 mic with EtOH at 20 ml/min; analysis | |
| | using 4.6 X 250 mm column; EtOH; 1 mL/min | |
| 16.3 | 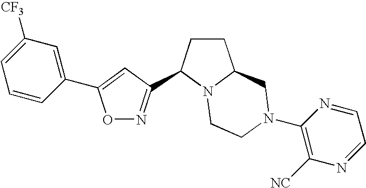 | 3-[(6R,8aS)-6-{5-[3-(trifluoromethyl)phenyl]isoxazol-3-yl}hexahydropyrrolo[1,2-a]pyrazin-2(1H)-yl]pyrazine-2-carbonitrile&3-[(6S,8aR)-6-{5-[3-(trifluoromethyl)phenyl]isoxazol-3-yl}hexahydropyrrolo[1,2-a]pyrazin-2(1H)-yl]pyrazine-2-carbonitrile |
| HPLC details | Fraction 1: RT 19.42 min, 99.78% purity, 40 mg, yellowish solid | |
| | Fraction 2: RT 27.09 min, 99.86% purity, 37 mg, yellowish solid | |
| | (Chiracel OJ, 20 mm × 250 mm, hexane/EtOH 70/30) | |
| 16.4 | 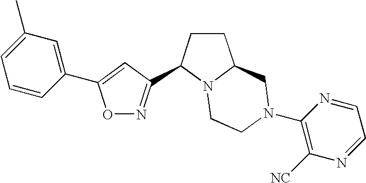 | 3-[(6R,8aS)-6-[5-(3-methylpheny1)isoxazol-3-yl]hexahydropyrrolo[1,2-a]pyrazin-2(1H)-yl]pyrazine-2-carbonitrile&3-[(6S,8aR)-6-[5-(3-methylphenyl)isoxazol-3-yl]hexahydropyrrolo[1,2-a]pyrazin-2(1H)-yl]pyrazine-2-carbonitrile |
| HPLC details | Fraction 1: RT 16.99 min, 97.57% purity, 61 mg, yellowish solid | |
| | Fraction 2: Rt 19.22 min, 99.80% purity, 62 mg, yellowish solid | |
| | (Chirapak AD, 20 mm × 250 mm, hexane/EtOH 40/60) | |
| 16.5 | 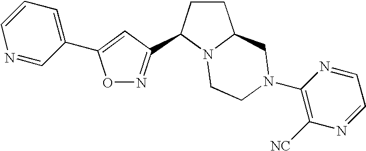 | 3-[(6R,8aS)-6-(5-pyridin-3-ylisoxazol-3-yl)hexahydropyrrolo[1,2-a]pyrazin-2(1H)-yl]pyrazine-2-carbonitrile&3-[(6S,8aR)-6-(5-pyridin-3-ylisoxazol-3-yl)hexahydropyrrolo[1,2-a]pyrazin-2(1H)-yl]pyrazine-2-carbonitrile |
| HPLC details | Fraction 1: RT 17.12 min, 95.52% purity, 20.8 mg, yellowish solid | |
| | Fraction 2: RT 28.64 min, 99.85% purity, 26.3 mg, yellowish solid | |
| | (Chiracel OJ, 20 mm × 250 mm, heptane/EtOH 40/60) | |
| 16.6 | 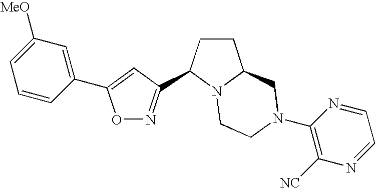 | 3-[(6R,8aS)-6-[5-(3-methoxyphenyl)isoxazol-3-yl]hexahydropyrrolol[1,2-a]pyrazin-2(1H)-yl]pyrazine-2-carbonitrile&3-[(6S,8aR)-6-[5-(3-methoxyphenyl)isoxazol-3-yl]hexahydropyrrolo[1,2-a]pyrazin-2(1H)-yl]pyrazine-2-carbonitrile |
| HPLC details | Fraction 1: RT 25.28 min, 99.98% purity, 53.6 mg, yellowish solid | |
| | Fraction 2: RT 31.20 min, 99.89% purity, 45.5 mg, yellowish solid | |
| | (Chiracel OJ, 20 mm × 250 mm, heptane/EtOH 40/60) | |
| 16.7 | 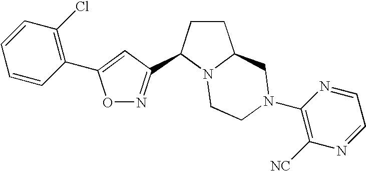 | 3-[(6R,8aS)-6-[5-(2-chlorophenyl)isoxazol-3-yl]hexahydropyrrolo[1,2-a]pyrazin-2(1H)-yl]pyrazine-2-carbonitrile&3-[(6S,8aR)-6-[5-(2-chlorophenyl)isoxazol-3-yl]hexahydropyrrolo[1,2-a]pyrazin-2(1H)-yl]pyrazine-2-carbonitrile |
| HPLC details | Fraction 1: RT 21.56 min, 100% purity, 28.1 mg, yellowish solid | |
| | Fraction 2: RT 27.16 min, 100% purity, 28.0 mg, yellowish solid | |
| | (Chirapak AD, 20 mm × 250 mm, hexane/EtOH 85/15) | |
| 16.8 | 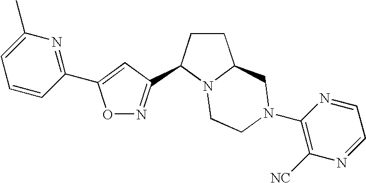 | 3-[(6R,8aS)-6-[5-(6-methylpyridin-2-yl)isoxazol-3-yl]hexahydropyrrolo[1,2-a]pyrazin-2(1H)-yl]pyrazine-2-carbonitrile&3-[(6S,8aR)-6-[5-(6-methylpyridin-2-yl)isoxazol-3-yl]hexahydropyrrolo[1,2-a]pyrazin-2(1H)-yl]pyrazine-2-carbonitrile |
| HPLC details | Fraction 1: RT 25.89 min, 99.59% purity, 30 mg, yellowish solid | |
| | Fraction 2: RT 29.20 min, 99.55% purity, 31 mg, yellowish solid | |
| | (Chiracel OJ, 20 mm × 250 mm, hexane/IPA/EtOH 80/15(05) | |
| 16.9 | 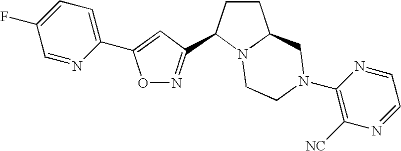 | 3-[(6R,8aS)-6-[5-(5-fluoropyridin-2-yl)isoxazol-3-yl]hexahydropyrrolo[1,2-a]pyrazin-2(1H)-yl]pyrazine-2-carbonitrile&3-[(6S,8aR)-6-[5-(5-fluoropyridin-2-yl)isoxazol-3-yl]hexahydropyrrolo[1,2-a]pyrazin-2(1H)-yl]pyrazine-2-carbonitrile |
| HPLC details | Fraction 1: RT 35.80 min, 99.79% purity, 5.2 mg, yellowish solid | |
| | Fraction 2: RT 47.70 min, 99.57% purity, 5.9 mg, yellowish solid | |
| | (Chiracel OJ, 20 mm × 250 mm, hexane/IPA/EtOH 80/15/05) | |
| 16.10 | 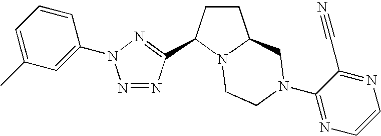 | 3-[(6R,8aS)-6-[2-(3-methylphenyl)-2H-tetrazol-5-yl]hexahydropyrrolol[1,2-a]pyrazin-2(1H)-yl]pyrazine-2-carbonitrile&3-[(6S,8aR)-6-[2-(3-methylphenyl)-2H-tetrazol-5-yl]hexahydropyrrolo[1,2-a]pyrazin-2(1H)-yl]pyrazine-2-carbonitrile |
| HPLC details | Fraction 1: pale-brown foam, 30 mg, 99.67%, Rt 44.504 min | |
| | Fraction 2: pale-brown foam, 29 mg, 99.55%, Rt 51.107 min | |
| | (Chiralpak AD; 20 mm × 250 mm, hexane/EtOH 95/5 at 25° C.) | |
| 16.11 | 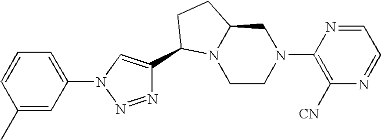 | 3-[(6R,8aS)-6-[1-(3-methylphenyl)-1H-1,2,3-triazol-4-yl]hexahydropyrrolo[1,2-a]pyrazin-2(1H)-yl]pyrazine-2-carbonitrile&3-[(6S,8aR)-6-[1-(3-methylphenyl)-1H-1,2,3-triazol-4-yl]hexahydropyrrolo(1,2-a]pyrazin-2(1H)-yl]pyrazine-2-carbonitrile |
| HPLC details | Fraction 1: RT 17.68 min, 99.96%, yellowish solid | |
| | Fraction 2: RT 29.79 min, 99.96%, yellowish solid | |
| | (Chiralpak_AD_20 mm X 250 mm, EtOH/100) | |
| 16.12 | 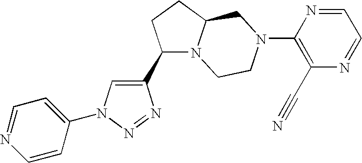 | 3-[(6R,8aS)-6-(1-pyridin-4-yl-1H-1,2,3-triazol-4-yl)hexahydropyrrolo[1,2-a]pyrazin-2(1H)-yl]pyrazine-2-carbonitrile&3-[(6S,8aR)-6-(1-pyridin-4-yl-1H-1,2,3-triazol-4-yl)hexahydropyrrolo[1,2-a]pyrazin-2(1H)-yl]pyrazine-2-carbonitrile |
| HPLC details | Fraction 1: RT 20.58 min, 100%, yellowish solid | |
| | Fraction 2: RT 34.47 min, 100%, yellowish solid | |
| | (Chiralpak_AD_20 mm X 250 mm, EtOH/100) | |
| 16.13 | 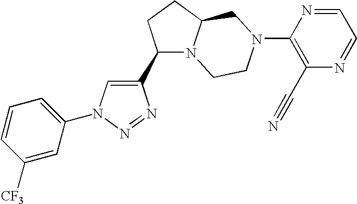 | 3-[(6R,8aS)-6-[1-(3-trifluoromethylphenyl)-1H-1,2,3-triazol-4-yl]hexahydropyrrolo[1,2-a]pyrazin-2(1H)-yl]pyrazine-2-carbonitrile&3-[(6S,8aR)-6-[1-(3-trifluoromethylphenyl)-1H-1,2,3-triazol-4-yl]hexahydropyrrolo[1,2-a]pyrazin-2(1H)-yl]pyrazine-2-carbonitrile |
| HPLC details | Fraction 1: RT 14.06 min, 97.11, yellowish solid | |
| | Fraction 2: RT 22.09 min, 100%,, yellowish solid | |
| | (Chiralpak_AD_20 mm X 250 mm, EtOH/100) | |
| 16.14 | 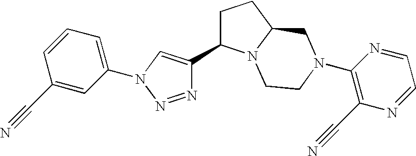 | 3-[(6R,8aS)-6-[1-(3-cyanophenyl)-1H-1,2,3-triazol-4-yl]hexahydropyrrolo[1,2-a]pyrazin-2(1H)-yl]pyrazine-2-carbonitrile&3-[(6S,8aR)-6-[1-(3-cyanophenyl)-1H-1,2,3-triazol-4-yl]hexahydropyrrolo[1,2-a]pyrazin-2(1H)-yl]pyrazine-2-carbonitrile |
| HPLC details | Fraction 1: RT 27.16 min, 100%, yellowish solid | |
| | Fraction 2: RT 32.37 min, 100%,, yellowish solid | |
| | (Chiralpak_AD_20 mm X 250 mm, EtOH/100) | |
| 16.15 | 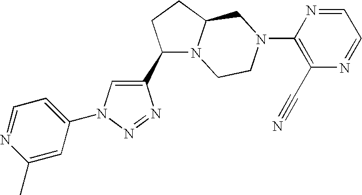 | 3-[(6R,8aS)-6-[1-(2-methylpyridin-4-yl)-1H-1,2,3-triazol-4-yl]hexahydropyrrolo[1,2-a]pyrazin-2(1H)-yl]pyrazine-2-carbonitrile&3-[(6S,8aR)-6-[1-(2-methylpyridin-4-yl)-1H-1,2,3-triazol-4-yl]hexahydropyrrolo[1,2-a]pyrazin-2(1H)-yl]pyrazine-2-carbonitrile |

| | |
|---|---|
| HPLC details | Fraction 1: RT 24.27 min, 99.86%, 71.31 mg, yellowish solid |
| | Fraction 2: RT 38.32 min, 99.86%, 95.61 mg, yellowish solid |
| | (Chiralcel_OH_20 mm X 250 mm, Hexane/EtOH 60/40) |

### Example 17

### Pharmaceutical Examples

Functional Assessment of mGluR5 Antagonism in Cell Lines Expressing mGluR5d

The properties of the compounds of the invention can be analyzed using standard assays for pharmacological activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

### FLIPR Assay

Cells expressing human mGluR5d as described in WO97/05252 are seeded at a density of 100,000 cells per well on collagen coated clear bottom 96-well plates with black sides and experiments are done 24 h following seeding. All assays are done in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). Cell cultures in the 96-well plates are loaded for 60 minutes in the above mentioned buffer containing 4 μM of the acetoxymethyl ester form of the fluorescent calcium indicator fluo-3 (Molecular Probes, Eugene, Oreg.) in 0.01% pluronic acid (a proprietary, non-ionic surfactant polyol—CAS Number 9003-11-6). Following the loading period the fluo-3 buffer is removed and replaced with fresh assay buffer. FLIPR experiments are done using a laser setting of 0.800 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 90 second interval separates the antagonist and agonist additions. The fluorescence signal is sampled 50 times at 1 second intervals followed by 3 samples at 5 second intervals immediately after each of the two additions. Responses are measured as the difference between the peak height of the response to agonist, less the background fluorescence within the sample period. IC₅₀ determinations are made using a linear least squares fitting program.

### IP3 Assay

An additional functional assay for mGluR5d is described in WO97/05252 and is based on phosphatidylinositol turnover. Receptor activation stimulates phospholipase C activity and leads to increased formation of inositol 1,4,5, triphosphate (IP₃).

GHEK stably expressing the human mGluR5d are seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells are washed once in HEPES buffered saline and pre-incubated for 10 min in HEPES buffered saline containing 10 mM LiCl. Compounds are incubated in duplicate at 37° C. for 15 min, then either glutamate (80 μM) or DHPG (30 μM) is added and incubated for an additional 30 min. The reaction is terminated by the addition of 0.5 ml perchloric acid (5%) on ice, with incubation at 4° C. for at least 30 min. Samples are collected in 15 ml polypropylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 ml 30 mM ammonium formate. Next, total inositol phosphates is eluted with 8 ml 700 mM ammonium formate/100 mM formic acid and collected in scintillation vials. This eluate is then mixed with 8 ml of scintillant and [3H] inositol incorporation is determined by scintillation counting. The dpm counts from the duplicate samples are plotted and IC₅₀ determinations are generated using a linear least squares fitting program.

Generally, the compounds of the present invention were active in the assays described herein at concentrations (or with IC₅₀ values) of less than 10 μM. Preferred compounds of the invention have IC₅₀ values of less than 1 μM; more preferred compounds of less than about 100 nM. For example, the compounds of Examples 4.1, 6.2, 13.1, 8.1, 5.1 and 5.4 have EC₅₀ values of 219, 2410, 159, 377, 10 and 16 nM, respectively.

What is claimed is:

1. A compound of formula I: 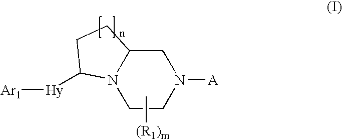
wherein
Ar₁ is an optionally-substituted aryl or heteroaryl group, wherein the substituents are selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R², SO₂R², aryl, heteroaryl, cycloalkyl and heterocycloalkyl, wherein any cyclic group may be further substituted with at least one substituent selected from the group consisting of F, Cl, Br, I, OH, nitro, C₁₋₆-alkyl, C₁₋₆-alkylhalo, OC₁₋₆-alkyl, OC₁₋₆-alkylhalo, C₂₋₆-alkenyl, C₂₋₆-alkynyl, CN, CO₂R², SR², S(O)R² and SO₂R²;
A is selected from the group consisting of Ar₁, CO₂R², CONR²R³, S(O)R² and SO₂R²;
R₁, in each instance, is independently selected from the group consisting of F, Cl, Br, I, OH, CN, nitro, C₁₋₆-alkyl, OC₁₋₆-alkyl, C₁₋₆-alkylhalo, OC₁₋₆-alkylhalo, (CO)R², O(CO)R², O(CO)OR², CO₂R², —CONR²R³, C₁₋₆-alkyleneOR², OC₂₋₆-alkyleneOR² and C₁₋₆-alkylenecyano;
R² and R³ are independently selected from the group consisting of H, C₁₋₆-alkyl, C₁₋₆-alkylhalo, C₂₋₆-alkenyl, C₂₋₆-alkynyl and cycloalkyl;
Hy is a 5-membered heterocyclic ring containing two, three or four heteroatoms independently selected from the group consisting of N, O and S, wherein the ring is optionally substituted with one or more substituents selected from the group consisting of F, Cl, Br, I, OH, nitro, C₁₋₆-alkyl, C₁₋₆-alkylhalo, OC₁₋₆-alkyl, OC₁₋₆-alkylhalo, CN, CO₂R², NR²R³, SR², S(O)R² and SO₂R²;
m is an integer selected from the group consisting of 0, 1, 2, 3 and 4; and
n is an integer selected from the group consisting of 1, 2 and 3;
or a pharmaceutically acceptable salt, hydrate, solvate, isoform, tautomer, optical isomer, or combination thereof.

2. A compound according to claim 1 wherein Ar₁ is an optionally-substituted phenyl group.

3. A compound according to claim 2 wherein A is selected from the group consisting of an optionally-substituted pyridyl group and an optionally-substituted pyrazinyl group.

4. A compound according to claim 3 wherein A is selected from the group consisting of an optionally-substituted 2-pyridyl group and an optionally-substituted 2-pyrazinyl group.

5. A compound according to claim 4 wherein Hy is selected from the group consisting of oxazole, isoxazole, 1,2,4-oxadiazole and 1,2,3-triazole

6. A compound selected from the group consisting of:
(±)-2-[(6R,8aS)-6-[1-(3-fluorophenyl)-1H-1,2,3-triazol-4-yl]hexahydropyrrolo[1,2-a]pyrazin-2(1H)-yl]nicotinonitrile,
(±)-3-[(6R,8aS)-6-[1-(3-fluorophenyl)-1H-1,2,3-triazol-4-yl]hexahydropyrrolo[1,2-a]pyrazin-2(1H)-yl]pyrazine-2-carbonitrile,
(±)-2-[(6R,8aS)-6-[1-(3-chlorophenyl)-1H-1,2,3-triazol-4-yl]hexahydropyrrolo[1,2-a]pyrazin-2(1H)-yl]nicotinonitrile,
(±)-3-[(6R,8aS)-6-[1-(3-chlorophenyl)-1H-1,2,3-triazol-4-yl]hexahydropyrrolo[1,2-a]pyrazin-2(1H)-yl]pyrazine-2-carbonitrile,
(±)-2-[(6R,8aS)-6-[1-(3-bromophenyl)-1H-1,2,3-triazol-4-yl]hexahydropyrrolo[1,2-a]pyrazin-2(1H)-yl]nicotinonitrile,
(±)-3-[(6R,8aS)-6-[1-(3-bromophenyl)-1H-1,2,3-triazol-4-yl]hexahydropyrrolo[1,2-a]pyrazin-2(1H)-yl]pyrazine-2-carbonitrile,
(±)-2-[(6R,8aS)-6-[1-(3-cyanophenyl)-1H-1,2,3-triazol-4-yl]hexahydropyrrolo[1,2-a]pyrazin-2(1H)-yl]nicotinonitrile,
(±)-3-[(6R,8aS)-6-[1-(3-cyanophenyl)-1H-1,2,3-triazol-4-yl]hexahydropyrrolo[1,2-a]pyrazin-2(1H)-yl]pyrazine-2-carbonitrile,
(±)-3-[(6R,8aS)-6-[2-(3-chlorophenyl)-2H-tetrazol-5-yl]hexahydropyrrolo[1,2-a]pyrazin-2(1H)-yl]pyrazine-2-carbonitrile,
(±)-2-[(6R,8aS)-6-[2-(3-chlorophenyl)-2H-tetrazol-5-yl]hexahydropyrrolo[1,2-a]pyrazin-2(1H)-yl]nicotinonitrile,
(±)-3-[(6R,8aS)-6-[2-(3-methylphenyl)-2H-tetrazol-5-yl]hexahydropyrrolo[1,2-a]pyrazin-2(1H)-yl]pyrazine-2-carbonitrile,
(±)-2-[(6R,8aS)-6-[2-(3-methylphenyl)-2H-tetrazol-5-yl]hexahydropyrrolo[1,2-a]pyrazin-2(1H)-yl]nicotinonitrile,
(±)-3-[(6R,8aS)-6-[5-(3-chlorophenyl)isoxazol-3-yl]hexahydropyrrolo[1,2-a]pyrazin-2(1H)-yl]pyrazine-2-carbonitrile,
(±)-6-[(6R,8aS)-6-[5-(3-chlorophenyl)isoxazol-3-yl]hexahydropyrrolo[1,2-a]pyrazin-2(1H)-yl]nicotinonitrile,
(±)-2-[(6R,8aS)-6-[5-(3-chlorophenyl)isoxazol-3-yl]hexahydropyrrolo[1,2-a]pyrazin-2(1H)-yl]nicotinonitrile,
(±)-3-[(6R,8aS)-6-{5-[3-(trifluoromethyl)phenyl]isoxazol-3-yl}hexahydropyrrolo[1,2-a]pyrazin-2(1H)-yl]pyrazine-2-carbonitrile,
(±)-3-[(6R,8aS)-6-[5-(3-methylphenyl)isoxazol-3-yl]hexahydropyrrolo[1,2-a]pyrazin-2(1H)-yl]pyrazine-2-carbonitrile,
(±)-3-[(6R,8aS)-6-(5-pyridin-3-ylisoxazol-3-yl)hexahydropyrrolo[1,2-a]pyrazin-2(1H)-yl]pyrazine-2-carbonitrile,
(±)-3-[(6R,8aS)-6-[5-(3-methoxyphenyl)isoxazol-3-yl]hexahydropyrrolo[1,2-a]pyrazin-2(1H)-yl]pyrazine-2-carbonitrile,
(±)-3-[(6R,8aS)-6-[5-(2-chlorophenyl)isoxazol-3-yl]hexahydropyrrolo[1,2-a]pyrazin-2(1H)-yl]pyrazine-2-carbonitrile,
(±)-3-[(6R,8aS)-6-[5-(6-methylpyridin-2-yl)isoxazol-3-yl]hexahydropyrrolo[1,2-a]pyrazin-2(1H)-yl]pyrazine-2-carbonitrile,
(±)-3-[(6R,8aS)-6-[5-(5-fluoropyridin-2-yl)isoxazol-3-yl]hexahydropyrrolo[1,2-a]pyrazin-2(1H)-yl]pyrazine-2-carbonitrile,
(±)-Ethyl (6R,8aS)-6-[5-(3-chlorophenyl)isoxazol-3-yl]hexahydropyrrolo[1,2-a]pyrazine-2(1H)-carboxylate,
(±)-3-[(6R,8aS)-6-[3-(3-chlorophenyl)isoxazol-5-yl]hexahydropyrrolo[1,2-a]pyrazin-2(1H)-yl]pyrazine-2-carbonitrile,
(±)-6-[(6R,9aS)-6-[5-(3-chlorophenyl)isoxazol-3-yl]octahydro-2H-pyrido[1,2-a]pyrazin-2-yl]pyrazine-2-carbonitrile,
(±)-6-[(6R,9aS)-6-[5-(3-cyanophenyl)isoxazol-3-yl]octahydro-2H-pyrido[1,2-a]pyrazin-2-yl]pyrazine-2-carbonitrile,
(±)-2-[(6R,9aS)-6-[3-(3-chlorophenyl)isoxazol-5-yl]octahydro-2H-pyrido[1,2-a]pyrazin-2-yl]nicotinonitrile,
(±)-2-[(6R,9aS)-6-[3-(3-cyanophenyl)isoxazol-5-yl]octahydro-2H-pyrido[1,2-a]pyrazin-2-yl]nicotinonitrile,
(±)-2-[(6R,8aS)-6-[1-(3-chlorophenyl)-1H-1,2,3-triazol-4-yl]hexahydropyrrolo[1,2-a]pyrazin-2(1H)-yl]-5-fluoronicotinonitrile,
(±)-6-[(6R,9aS)-6-[5-(3-cyanophenyl)isoxazol-3-yl]hexahydropyrrolo[1,2-a]pyrazin-2-yl]-5-fluoronicotinonitrile,
(±)-2-[(6R,9aS)-6-[5-(3-chlorophenyl)isoxazol-3-yl]octahydro-2H-pyrido[1,2-a]pyrazin-2(1H)-yl]nicotinonitrile,
(±)-6-[(6R,9aS)-6-[5-(3-cyanophenyl)-1,2,3-triazol-4-yl]octahydro-2H-pyrido[1,2-a]pyrazin-2-yl]pyrazine-2-carbonitrile,
(±)-6-[(6R,9aS)-6-[5-(3-chloro-phenyl)-1,2,3-triazol-4-yl]octahydro-2H-pyrido[1,2-a]pyrazin-2-yl]pyrazine-2-carbonitrile,
(±)-6-[(6R,9aS)-6-[5-(3-cyanophenyl)-1,2,3-triazol-4-yl]octahydro-2H-pyrido[1,2-a]pyrazin-2(1H)-yl]nicotinonitrile,
(±)-6-[(6R,9aS)-6-[5-(3-chloro-phenyl)-1,2,3-triazol-4-yl]octahydro-2H-pyrido[1,2-a]pyrazin-2(1H)-yl]nicotinonitrile,
(±)-3-[(6R,9aS)-6-[5-(3-cyanophenyl)isoxazol-3-yl]octahydro-2H-pyrido[1,2-a]pyrazin-2-yl]pyrazine-2-carbonitrile,
(±)-3-[(6R,9aS)-6-[5-(3-chlorophenyl)isoxazol-3-yl]octahydro-2H-pyrido[1,2-a]pyrazin-2-yl]pyrazine-2-carbonitrile,
(±)-2-[(6R,9aS)-6-[5-(3-chlorophenyl)isoxazol-3-yl]octahydro-2H-pyrido[1,2-a]pyrazin-2-yl]nicotinonitrile,
(±)-2-[(6R,9aS)-6-[5-(6-methylpyrid-2-yl)isoxazol-3-yl]octahydro-2H-pyrido[1,2-a]pyrazin-2-yl]nicotinonitrile,
(±)-6-[(6R,9aS)-6-[5-(pyrid-2-yl)isoxazol-3-yl]octahydro-2H-pyrido[1,2-a]pyrazin-2-yl]pyrazine-2-carbonitrile,
(±)-3-[(6R,8aS)-6-[1-(3-methylphenyl)-1H-1,2,3-triazol-4-yl]hexahydropyrrolo[1,2-a]pyrazin-2(1H)-yl]pyrazine-2-carbonitrile,
(±)-3-[(6R,8aS)-6-(1-pyridin-4-yl-1H-1,2,3-triazol-4-yl)hexahydropyrrolo[1,2-a]pyrazin-2(1H)-yl]pyrazine-2-carbonitrile,
(±)-3-[(6R,8aS)-6-[1-(3-trifluoromethylphenyl)-1H-1,2,3-triazol-4-yl]hexahydropyrrolo[1,2-a]pyrazin-2(1H)-yl]pyrazine-2-carbonitrile,
(±)-3-[(6R,8aS)-6-[1-(2-methylpyridin-4-yl)-1H-1,2,3-triazol-4-yl]hexahydropyrrolo[1,2-a]pyrazin-2(1H)-yl]pyrazine-2-carbonitrile,
3-[(6R,8aS)-6-[5-(3-chlorophenyl)isoxazol-3-yl]hexahydropyrrolo[1,2-a]pyrazin-2(1H)-yl]pyrazine-2-carbonitrile,
3-[(6S,8aR)-6-[5-(3-chlorophenyl)isoxazol-3-yl]hexahydropyrrolo[1,2-a]pyrazin-2(1H)-yl]pyrazine-2-carbonitrile,
3-[(6R,8aS)-6-[1-(3-chlorophenyl)-1H-1,2,3-triazol-4-yl]hexahydropyrrolo[1,2-a]pyrazin-2(1H)-yl]pyrazine-2-carbonitrile,
3-[(6S,8aR)-6-[1-(3-chlorophenyl)-1H-1,2,3-triazol-4-yl]hexahydropyrrolo[1,2-a]pyrazin-2(1H)-yl]pyrazine-2-carbonitrile,
3-[(6R,8aS)-6-{5-[3-(trifluoromethyl)phenyl]isoxazol-3-yl}hexahydropyrrolo[1,2-a]pyrazin-2(1H)-yl]pyrazine-2-carbonitrile,
3-[(6S,8aR)-6-{5-[3-(trifluoromethyl)phenyl]isoxazol-3-yl}hexahydropyrrolo[1,2-a]pyrazin-2(1H)-yl]pyrazine-2-carbonitrile,
3-[(6R,8aS)-6-[5-(3-methylphenyl)isoxazol-3-yl]hexahydropyrrolo[1,2-a]pyrazin-2(1H)-yl]pyrazine-2-carbonitrile,
3-[(6S,8aR)-6-[5-(3-methylphenyl)isoxazol-3-yl]hexahydropyrrolo[1,2-a]pyrazin-2(1H)-yl]pyrazine-2-carbonitrile,
3-[(6R,8aS)-6-(5-pyridin-3-ylisoxazol-3-yl)hexahydropyrrolo[1,2-a]pyrazin-2(1H)-yl]pyrazine-2-carbonitrile,
3-[(6S,8a)-6-(5-pyridin-3-yl)isoxazol-3-yl)hexahydropyrrolo[1,2-a]pyrazin-2(1H)-yl]pyrazine-2-carbonitrile,
3-[(6R,8aS)-6-[5-(3-methoxyphenyl)isoxazol-3-yl]hexahydropyrrolo[1,2-a]pyrazin-2(1H)-yl]pyrazine-2-carbonitrile,
3-[(6S,8aR)-6-[5-(3-methoxyphenyl)isoxazol-3-yl]hexahydropyrrolo[1,2-a]pyrazin-2(1H)-yl]pyrazine-2-carbonitrile,
3-[(6R,8aS)-6-[5-(2-chlorophenyl)isoxazol-3-yl]hexahydropyrrolo[1,2-a]pyrazin-2(1H)-yl]pyrazine-2-carbonitrile,
3-[(6S,8a)-6-[5-(2-chlorophenyl)isoxazol-3-yl]hexahydropyrrolo[1,2-a]pyrazin-2(1B)-yl]pyrazine-2-carbonitrile,
3-[(6R,8aS)-6-[5-(6-methylpyridin-2-yl)isoxazol-3-yl]hexahydropyrrolo[1,2-a]pyrazin-2(1H)-yl]pyrazine-2-carbonitrile,
3-[(6S,8aR)-6-[5-(6-methylpyridin-2-yl)isoxazol-3-yl]hexahydropyrrolo[1,2-a]pyrazin-2(1H)-yl]pyrazine-2-carbonitrile,
3-[(6R,8aS)-6-(5-(5-fluoropyridin-2-yl)isoxazol-3-yl]hexahydropyrrolo[1,2-a]pyrazin-2(1H)-yl]pyrazine-2-carbonitrile,
3-[(6S,8a)-6-(5-(5-fluoropyridin-2-yl)isoxazol-3-yl]hexahydropyrrolo[1,2-a]pyrazin-2(1H)-yl]pyrazine-2-carbonitrile,
3-[(6R,8aS)-6-[2-(3-methylphenyl)-2H-tetrazol-5-yl]hexahydropyrrolo[1,2-a]pyrazin-2(1H)-yl]pyrazine-2-carbonitrile,
3-[(6S,8aR)-6-[2-(3-methylphenyl)-2H-tetrazol-5-yl]hexahydropyrrolo[1,2-a]pyrazin-2(1H)-yl]pyrazine-2-carbonitrile,
3-[(6R,8aS)-6-[1-(3-methylphenyl)-1H-1,2,3-triazol-4-yl]hexahydropyrrolo[1,2-a]pyrazin-2(1H)-yl]pyrazine-2-carbonitrile,
3-[(6S,8aR)-6-[1-(3-methylphenyl)-1H-1,2,3-triazol-4-yl]hexahydropyrrolo[1,2-a]pyrazin-2(1H)-yl]pyrazine-2-carbonitrile,
3-[(6R,8aS)-6-(1-pyridin-4-yl-1H-1,2,3-triazol-4-yl)hexahydropyrrolo[1,2-a]pyrazin-2(1H)-yl]pyrazine-2-carbonitrile,
3-[(6S,8aR)-6-(1-pyridin-4-yl-1H-1,2,3-triazol-4-yl)hexahydropyrrolo[1,2-a]pyrazin-2(1H)-yl]pyrazine-2-carbonitrile,
3-[(6R,8aS)-6-[1-(3-trifluoromethylphenyl)-1H-1,2,3-triazol-4-yl]hexahydropyrrolo[1,2-a]pyrazin-2(1H)-yl]pyrazine-2-carbonitrile,
3-[(6S,8aR)-6-[1-(3-trifluoromethylphenyl)-1H-1,2,3-triazol-4-yl]hexahydropyrrolo[1,2-a]pyrazin-2(1H)-yl]pyrazine-2-carbonitrile,
3-[(6R,8aS)-6-[1-(3-cyanophenyl)-1H-1,2,3-triazol-4-yl]hexahydropyrrolo[1,2-a]pyrazin-2(1H)-yl]pyrazine-2-carbonitrile,
3-[(6S,8aR)-6-[1-(3-cyanophenyl)-1H-1,2,3-triazol-4-yl]hexahydropyrrolo[1,2-a]pyrazin-2(1H)-yl]pyrazine-2-carbonitrile,
3-[(6R,8aS)-6-[1-(2-methylpyridin-4-yl)-1H-1,2,3-triazol-4-yl]hexahydropyrrolo[1,2-a]pyrazin-2(1H)-yl]pyrazine-2-carbonitrile and
3-[(6S,8aR)-6-[1-(2-methylpyridin-4-yl)-1H-1,2,3-triazol-4-yl]hexahydropyrrolo[1,2-a]pyrazin-2(1H)-yl]pyrazine-2-carbonitrile.

7. A pharmaceutical composition comprising as active ingredient a therapeutically effective amount of the compound according to claim 1, in association with one or more pharmaceutically acceptable diluents, excipients and/or inert carriers.

8. The pharmaceutical composition according to claim 7, for use in the treatment of mGluR5 mediated disorders.

9. The compound according to claim 1 for use in therapy.

10. The compound according to claim 1 for use in treatment of mGluR5-mediated disorders.

11. Use of the compound according to claim 1, in the manufacture of a medicament for the treatment of mGluR5-mediated disorders.

12. A method of treatment of mGluR5-mediated disorders, comprising administering to a mammal a therapeutically effective amount of the compound according to claim 1.

13. The method according to claim 12, wherein the mammal is a human.

14. The method according to claim 12, wherein the disorders are neurological disorders.

15. The method according to claim 12, wherein the disorders are psychiatric disorders.

16. The method according to claim 12, wherein the disorders are chronic and acute pain disorders.

17. The method according to claim 12, wherein the disorders are gastrointestinal disorders.

18. A method for inhibiting activation of mGluR5 receptors, comprising treating a cell containing said receptor with an effective amount of the compound according to claim 1.
